(19) United States
(12) Patent Application Publication
(10) Pub. No.: US 2024/0301374 A1
(43) Pub. Date: Sep. 12, 2024

(54) SYSTEMS AND METHODS FOR TRANSPOSING CARGO NUCLEOTIDE SEQUENCES

(71) Applicant: METAGENOMI, INC., Emeryville, CA (US)

(72) Inventors: Brian THOMAS, Emeryville, CA (US); Christopher BROWN, Emeryville, CA (US); Daniela S.A. GOLTSMAN, Emeryville, CA (US); Cristina BUTTERFIELD, Emeryville, CA (US); Lisa ALEXANDER, Emeryville, CA (US); Jason LIU, Emeryville, CA (US)

(21) Appl. No.: 18/481,769

(22) Filed: Oct. 5, 2023

Related U.S. Application Data
(63) Continuation of application No. 18/173,722, filed on Feb. 23, 2023, now abandoned.
Application No. 18/173,722 is a continuation of application No. PCT/US2021/047195, filed on Aug. 23, 2021.
(60) Provisional application No. 63/069,703, filed on Aug. 24, 2020.
(60) Provisional application No. 63/186,698, filed on May 10, 2021.
(60) Provisional application No. 63/232,593, filed on Aug. 12, 2021.

(51) Int. Cl. C12N 9/22; C12N 9/12; C12N 15/11

(52) U.S. Cl. CPC C12N 9/22; C12N 9/1241; C12N 15/11; C12N 2310/20

(57) ABSTRACT

The present disclosure provides systems and methods for transposing a cargo nucleotide sequence to a target nucleic acid site. These systems and methods may comprise a first double-stranded nucleic acid comprising the cargo nucleotide sequence, wherein the cargo nucleotide sequence is configured to interact with a recombinase or transposase complex, a cas effector complex comprising a cas effector and at least one engineered guide polynucleotide configured to hybridize to the target nucleic acid site, and the recombinase or transposase complex wherein said recombinase or transposase complex is configured to recruit the cargo nucleotide to the target nucleic acid site.

### RELATED APPLICATIONS

This application is a continuation of U.S. application Ser. No. 18/173,722, filed Feb. 23, 2023, entitled “SYSTEMS AND METHODS FOR TRANSPOSING CARGO NUCLEOTIDE SEQUENCES”, which is a continuation of International Patent Application No. PCT/US2021/047195, filed Aug. 23, 2021, entitled “SYSTEMS AND METHODS FOR TRANSPOSING CARGO NUCLEOTIDE SEQUENCES”, which claims the benefit of U.S. Provisional Application No. 63/069,703, filed Aug. 24, 2020, entitled “SYSTEMS AND METHODS FOR TRANSPOSING CARGO NUCLEOTIDE SEQUENCES”, U.S. Provisional Application No. 63/186,698, filed May 10, 2021, entitled “SYSTEMS AND METHODS FOR TRANSPOSING CARGO NUCLEOTIDE SEQUENCES”, and U.S. Provisional Application No. 63/232,593, filed Aug. 12, 2021, entitled “SYSTEMS AND METHODS FOR TRANSPOSING CARGO NUCLEOTIDE SEQUENCES”, each of which is incorporated in its entirety herein.

### BACKGROUND

Cas enzymes along with their associated Clustered Regularly Interspaced Short Palindromic Repeats (CRISPR) guide ribonucleic acids (RNAs) appear to be a pervasive (˜45% of bacteria, ˜84% of archaea) component of prokaryotic immune systems, serving to protect such microorganisms against non-self nucleic acids, such as infectious viruses and plasmids by CRISPR-RNA guided nucleic acid cleavage. While the deoxyribonucleic acid (DNA) elements encoding CRISPR RNA elements may be relatively conserved in structure and length, their CRISPR-associated (Cas) proteins are highly diverse, containing a wide variety of nucleic acid-interacting domains. While CRISPR DNA elements have been observed as early as 1987, the programmable endonuclease cleavage ability of CRISPR/Cas complexes has only been recognized relatively recently, leading to the use of recombinant CRISPR/Cas systems in diverse DNA manipulation and gene editing applications.

### Sequence Listing

The instant application contains a Sequence Listing which has been submitted electronically in XML format and is hereby incorporated by reference in its entirety. Said XML copy, created on Oct. 2, 2023, is named 55921-714.303.xml and is 524,408 bytes in size.

### SUMMARY

In some aspects, the present disclosure provides for a system for transposing a cargo nucleotide sequence to a target nucleic acid site comprising: a first double-stranded nucleic acid comprising said cargo nucleotide sequence, wherein said cargo nucleotide sequence is configured to interact with a recombinase or transposase complex; a Cas effector complex comprising a class II, type II Cas effector and at least one engineered guide polynucleotide configured to hybridize to said target nucleic acid site; and said recombinase or transposase complex, wherein said recombinase or transposase complex is configured to recruit said cargo nucleotide sequence to said target nucleic acid site. In some embodiments, said recombinase or transposase complex binds non-covalently to said Cas effector complex. In some embodiments, said recombinase or transposase complex is covalently linked to said Cas effector complex. In some embodiments, said recombinase or transposase complex is fused to said Cas effector complex in a single polypeptide. In some embodiments, said cargo nucleotide sequence is flanked by a left-hand transposase recognition sequence and a right-hand transposase recognition sequence. In some embodiments, the system further comprises a second double-stranded nucleic acid comprising said target nucleic acid site. In some embodiments, the system further comprises a PAM sequence compatible with said Cas effector complex adjacent to said target nucleic acid site. In some embodiments, said PAM sequence is located 3′ of said target nucleic acid site. In some embodiments, said recombinase or transposase complex is a Tn7 type transposase complex. In some embodiments, said engineered guide polynucleotide is configured to bind said class II, type II Cas effector. In some embodiments, said class II, type II Cas effector comprises a polypeptide comprising a sequence having at least 80% identity to SEQ ID NO: 1 or a variant thereof. In some embodiments, said recombinase or transposase complex comprises at least one, at least two, at least three, or four polypeptide(s) comprising a sequence having at least 80% identity to any one of SEQ ID NOs: 2-5 or a variant thereof. In some embodiments, said engineered guide polynucleotide comprises a sequence comprising at least 60-80 consecutive nucleotides having at least 80% identity to SEQ ID NO: 12 or a variant thereof. In some embodiments, said engineered guide polynucleotide comprises a sequence having at least 80% identity to SEQ ID NO: 11 or a variant thereof. In some embodiments, said left-hand recombinase sequence comprises a sequence having at least 80% identity to any one of SEQ ID NOs: 17-18 or a variant thereof. In some embodiments, said right-had recombinase sequence comprises a sequence having at least 80% identity to SEQ ID NO: 19 or a variant thereof. In some embodiments, said class II, type II Cas effector and said recombinase or transposase complex are encoded by polynucleotide sequences comprising fewer than about 10 kilobases

In some aspects, the present disclosure provides for a method for transposing a cargo nucleotide sequence to a target nucleic acid site comprising a target nucleotide sequence comprising expressing the system of any of the aspects or embodiments described herein within a cell or introducing the system of any of the aspects or embodiments described herein to a cell.

In some aspects, the present disclosure provides for a system for transposing a cargo nucleotide sequence to a target nucleic acid site comprising: a first double-stranded nucleic acid comprising a cargo nucleotide sequence configured to interact with a Tn7 type transposase complex; a Cas effector complex comprising a class II, type V Cas effector and an engineered guide polynucleotide configured to hybridize to said target nucleotide sequence; and a Tn7 type transposase complex configured to bind said Cas effector complex, wherein said Tn7 type transposase complex comprises a TnsA subunit. In some embodiments, said transposase complex binds non-covalently to said Cas effector complex. In some embodiments, said transposase complex is covalently linked to said Cas effector complex. In some embodiments, said transposase complex is fused to said Cas effector complex in a single polypeptide. In some embodiments, said class II, type V Cas effector is not a Cas12k effector. In some embodiments, said cargo nucleotide sequence is flanked by a left-hand transposase recognition sequence and a right-hand transposase recognition sequence. In some embodiments, the system further comprises a second double-stranded nucleic acid comprising said target nucleic acid site. In some embodiments, the system further comprises a PAM sequence compatible with said Cas effector complex adjacent to said target nucleic acid site. In some embodiments, said PAM sequence is located 5′ of said target nucleic acid site. In some embodiments, said engineered guide polynucleotide is configured to bind said class II, type V Cas effector. In some embodiments, said TnsA subunit comprises a polypeptide having a sequence having at least 80% identity to SEQ ID NO: 7 or a variant thereof. In some embodiments, said Tn7 type transposase complex comprises at least one, at least two, or three polypeptide(s) comprising a sequence having at least 80% identity to any one of SEQ ID NOs: 8-10, or a variant thereof. In some embodiments, said engineered guide polynucleotide comprises a sequence comprising at least about 46-80 consecutive nucleotides having at least 80% identity to any one of SEQ ID NOs: 13-16, or a variant thereof. In some embodiments, said left-hand recombinase sequence comprises a sequence having at least 80% identity to SEQ ID NO: 20, or a variant thereof. In some embodiments, said right-hand recombinase sequence comprises a sequence having at least 80% identity to SEQ ID NO: 21, or a variant thereof. In some embodiments, said class II, type V Cas effector is not a Cas12k effector. In some embodiments, said class II, type V Cas effector and said Tn7 type transposase complex are encoded by polynucleotide sequences comprising fewer than about 10 kilobases.

In some aspects, the present disclosure provides for a method for transposing a cargo nucleotide sequence to a target nucleic acid site comprising a target nucleotide sequence comprising expressing the system of any one of any of the aspects or embodiments described herein within a cell or introducing the system of any one of the aspects or embodiments described herein to a cell.

In some aspects, the present disclosure provides for a method for transposing a cargo nucleotide sequence to a target nucleic acid site, comprising contacting a first double-stranded nucleic acid comprising a cargo nucleotide sequence with: a Cas effector complex comprising a class II, type II Cas effector and at least one engineered guide polynucleotide configured to hybridize to said target nucleic acid site; a recombinase or transposase complex configured to recruit said cargo nucleotide to said target nucleic acid site; and a second double-stranded nucleic acid comprising said target nucleic acid site. In some embodiments, said recombinase or transposase complex binds non-covalently to said Cas effector complex. In some embodiments, said recombinase or transposase complex is covalently linked to said Cas effector complex. In some embodiments, said recombinase or transposase complex is fused to said Cas effector complex in a single polypeptide. In some embodiments, said cargo nucleotide sequence is flanked by a left-hand transposase recognition sequence and a right-hand transposase recognition sequence. In some embodiments, the target nucleic acid further comprises a PAM sequence compatible with said Cas effector complex adjacent to said target nucleic acid site. In some embodiments, said PAM sequence is located 3′ of said target nucleic acid site. In some embodiments, said recombinase or transposase complex is a Tn7 type transposase complex. In some embodiments, said engineered guide polynucleotide is configured to bind said class II, type II Cas effector. In some embodiments, said class II, type II Cas effector comprises a polypeptide comprising a sequence having at least 80% identity to SEQ ID NO: 1 or a variant thereof. In some embodiments, said recombinase or transposase complex comprises at least one, at least two, at least three, or four polypeptide(s) comprising a sequence having at least 80% identity to any one of SEQ ID NOs: 2-5 or a variant thereof. In some embodiments, said engineered guide polynucleotide comprises a sequence comprising at least 60-80 consecutive nucleotides having at least 80% identity to SEQ ID NO: 12 or a variant thereof. In some embodiments, said engineered guide polynucleotide comprises a sequence having at least 80% identity to SEQ ID NO: 11 or a variant thereof. In some embodiments, said left-hand recombinase sequence comprises a sequence having at least 80% identity to any one of SEQ ID NOs: 17-18 or a variant thereof. In some embodiments, said right-hand recombinase sequence comprises a sequence having at least 80% identity to SEQ ID NO: 19 or a variant thereof. In some embodiments, said class II, type II Cas effector and said Tn7 type transposase complex are encoded by polynucleotide sequences comprising fewer than about 10 kilobases.

In some aspects, the present disclosure provides for a method for transposing a cargo nucleotide sequence to a target nucleic acid site, comprising contacting a first double-stranded nucleic acid comprising said cargo nucleotide sequence with: a Cas effector complex comprising a class II, type V Cas effector and at least one engineered guide polynucleotide configured to hybridize to said target nucleotide sequence; a Tn7 type transposase complex configured to bind said Cas effector complex, wherein said Tn7 type transposase complex comprises a TnsA subunit; and a second double-stranded nucleic acid comprising said target nucleic acid site. In some embodiments, said transposase complex binds non-covalently to said Cas effector complex. In some embodiments, said transposase complex is covalently linked to said Cas effector complex. In some embodiments, said transposase complex is fused to said Cas effector complex in a single polypeptide. In some embodiments, said cargo nucleotide sequence is flanked by a left-hand transposase recognition sequence and a right-hand transposase recognition sequence. In some embodiments, said target nucleic acid site further comprises a PAM sequence compatible with said Cas effector complex adjacent to said target nucleic acid site. In some embodiments, said PAM sequence is located 3′ of said target nucleic acid site. In some embodiments, said engineered guide polynucleotide is configured to bind said class II, type V Cas effector. In some embodiments, said TnsA subunit comprises a polypeptide having a sequence having at least 80% identity to SEQ ID NO: 7 or a variant thereof. In some embodiments, said Tn7 type transposase complex comprises at least one, at least two, or three polypeptide(s) comprising a sequence having at least 80% identity to any one of SEQ ID NOs: 8-10, or a variant thereof. In some embodiments, said engineered guide polynucleotide comprises a sequence comprising at least about 46-80 consecutive nucleotides having at least 80% identity to any one of SEQ ID NOs: 13-16 or a variant thereof. In some embodiments, said left-hand recombinase sequence comprises a sequence having at least 80% identity to SEQ ID NO: 20, or a variant thereof. In some embodiments, said right-hand recombinase sequence comprises a sequence having at least 80% identity to SEQ ID NO: 21, or a variant thereof. In some embodiments, said class II, type V Cas effector is not a Cas12k effector. In some embodiments, said class II, type V Cas effector and said Tn7 type transposase complex are encoded by polynucleotide sequences comprising fewer than about 10 kilobases.

In some aspects, the present disclosure provides for a system for transposing a cargo nucleotide sequence to a target nucleic acid site comprising: a first double-stranded nucleic acid comprising a cargo nucleotide sequence configured to interact with a Tn7 type transposase complex; a Cas effector complex comprising a class I, type I-F Cas effector and an engineered guide polynucleotide configured to hybridize to said target nucleotide sequence; and a Tn7 type transposase complex configured to bind said Cas effector complex, wherein said Tn7 type transposase complex comprises a TnsA subunit. In some embodiments, said transposase complex binds non-covalently to said Cas effector complex. In some embodiments, said transposase complex is covalently linked to said Cas effector complex. In some embodiments, said transposase complex is fused to said Cas effector complex in a single polypeptide. In some embodiments, said cargo nucleotide sequence is flanked by a left-hand transposase recognition sequence and a right-hand transposase recognition sequence. In some embodiments, the system further comprises a second double-stranded nucleic acid comprising said target nucleic acid site. In some embodiments, the system further comprises a PAM sequence compatible with said Cas effector complex adjacent to said target nucleic acid site. In some embodiments, said PAM sequence is located 3′ of said target nucleic acid site. In some embodiments, said PAM sequence is located 5′ of said target nucleic acid site. In some embodiments, said engineered guide polynucleotide is configured to bind said class I, type I-F Cas effector. In some embodiments, said class I, type I-F Cas effector comprises a polypeptide comprising a sequence having at least 80% identity to any one of SEQ ID NO: 41-43, or 48-50, or a variant thereof. In some embodiments, said Tn7 type transposase complex comprises at least one, at least two, or three polypeptide(s) comprising a sequence having at least 80% identity to any one of SEQ ID NOs: 44-46, or 51-53, or a variant thereof.

In some aspects, the present disclosure provides for a method for transposing a cargo nucleotide sequence to a target nucleic acid site comprising a target nucleotide sequence comprising expressing the system of any one of the aspects or embodiments described herein within a cell or introducing the system of any one of the aspects or embodiments described herein to a cell.

In some aspects, the present disclosure provides for a system for transposing a cargo nucleotide sequence to a target nucleic acid site comprising: a first double-stranded nucleic acid comprising a cargo nucleotide sequence configured to interact with a Tn7 type transposase complex; a Cas effector complex comprising a class II, type V Cas effector and an engineered guide polynucleotide configured to hybridize to said target nucleotide sequence; and a Tn7 type transposase complex configured to bind said Cas effector complex, wherein said Tn7 type transposase complex comprises TnsB, TnsC, and TniQ components, wherein: (a) said class II, type V Cas effector comprises a polypeptide having a sequence having at least 80% sequence identity to any one of SEQ ID NOs:22, 26, 30, 34, 55-89, 104, or 147, or a variant thereof; or (b) said Tn7 type transposase complex comprises a TnsB, TnsC, or TniQ component having a sequence having at least 80% sequence identity to any one of SEQ ID NOs:23-25, 27-29, 31-33, 35-37, 101-103, 105-107, or 148-150, or a variant thereof. In some embodiments, said transposase complex binds non-covalently to said Cas effector complex. In some embodiments, said transposase complex is covalently linked to said Cas effector complex. In some embodiments, said transposase complex is fused to said Cas effector complex in a single polypeptide. In some embodiments, said class II, type V Cas effector comprises a polypeptide comprising a sequence having at least 80% sequence identity to any one of SEQ ID NOs:22, 26, 30, 34, 55-89, 104, or 147, or a variant thereof. In some embodiments, said Tn7 type transposase complex comprises a TnsB, TnsC, or TniQ component comprising a sequence having at least 80% sequence identity to any one of SEQ ID NOs:23-25, 27-29, 31-33, 35-37, 101-103, 105-107, or 148-150, or a variant thereof. In some embodiments, said class II, type V Cas effector is a Cas12k effector. In some embodiments, said cargo nucleotide sequence is flanked by a left-hand transposase recognition sequence and a right-hand transposase recognition sequence. In some embodiments, the system further comprises a second double-stranded nucleic acid comprising said target nucleic acid site. In some embodiments, the system further comprises a PAM sequence compatible with said Cas effector complex adjacent to said target nucleic acid site. In some embodiments, said PAM sequence is located 5′ of said target nucleic acid site. In some embodiments, said PAM sequence comprises 5′-nGTn-3′ or 5′-nGTt-3′. In some embodiments, said engineered guide polynucleotide is configured to bind said class II, type V Cas effector. In some embodiments, said TnsB, TnsC, and TniQ components comprise polypeptides having a sequence having at least 80% identity to any one of SEQ ID NOs:23-25, 27-29, 31-33, 35-37, 101-103, 105-107, or 148-150, respectively. In some embodiments, said engineered guide polynucleotide comprises a sequence comprising at least about 46-80 consecutive nucleotides having at least 80% identity to any one of SEQ ID NOs: 90, 91, 92, 93, 117, 151, 156-181, or 209-234. In some embodiments, said engineered guide polynucleotide comprises a sequence having at least 80% sequence identity to any one of SEQ ID NOs: 111-114 or 201-206, 255, 262, 256, 209, 257, 263, 258, 210, or a variant thereof. In some embodiments, said left-hand recombinase sequence comprises a sequence having at least 80% identity to any one of SEQ ID NOs: 125, 127, 123, 129, 131, 133, 153, or 134, or a variant thereof. In some embodiments, said right-hand recombinase sequence comprises a sequence having at least 80% identity to any one of SEQ ID NOs: 126, 155, 128, 124, 130, 132, or 154, or a variant thereof. In some embodiments, said class II, type V Cas effector and said Tn7 type transposase complex are encoded by polynucleotide sequences comprising fewer than about 10 kilobases. In some embodiments: (a) said class II, type V Cas effector comprises a sequence having at least 80% sequence identity to SEQ ID NO:22 or a variant thereof; (b) said left-hand recombinase sequence comprises a sequence having at least 80% sequence identity to SEQ ID NO:125 or a variant thereof; (c) said right-hand recombinase sequence comprises a sequence having at least 80% identity to SEQ ID NO: 126 or 155, or a variant thereof; (d) said engineered guide polynucleotide: (i) comprises a sequence having at least 80% sequence identity to at least about 46-60 nucleotides of SEQ ID NO: 90; or (ii) comprises a sequence having at least 80% sequence identity to non-degenerate nucleotides of any one of SEQ ID NOs: 94, 112, or 202; or (e) said TnsB, TnsC, and TniQ components comprise sequences having at least 80% sequence identity to SEQ ID NOs: 23-25 or variants thereof. In some embodiments: (a) said class II, type V Cas effector comprises a sequence having at least 80% sequence identity to SEQ ID NO:26 or a variant thereof; (b) said left-hand recombinase sequence comprises a sequence having at least 80% sequence identity to SEQ ID NO:127 or a variant thereof; (c) said right-hand recombinase sequence comprises a sequence having at least 880% sequence identity to SEQ ID NO: 128 or a variant thereof; (d) said engineered guide polynucleotide: (i) comprises a sequence having at least 80% sequence identity to at least about 46-60 nucleotides of any one of SEQ ID NOs: 91, 156, or 209; or (ii) comprises a sequence having at least 80% sequence identity to non-degenerate nucleotides of any one of SEQ ID NOs: 95, 113, or 203, or (e) said TnsB, TnsC, and TniQ components comprise sequences having at least 80% sequence identity to SEQ ID NOs: 27-29 or variants thereof. In some embodiments: (a) said class II, type V Cas effector comprises a sequence having at least 80% sequence identity to SEQ ID NO:60 or a variant thereof; (b) said left-hand recombinase sequence comprises a sequence having at least 80% sequence identity to SEQ ID NO: 131 or a variant thereof; (c) said right-hand recombinase sequence comprises a sequence having at least 80% sequence identity to SEQ ID NO: 132 or a variant thereof; (d) said engineered guide polynucleotide: (i) comprises a sequence having at least 80% sequence identity to at least about 46-60 nucleotides of any one of SEQ ID NOs: 117, 161, or 214; or (ii) comprises a sequence having at least 80% sequence identity to non-degenerate nucleotides of SEQ ID NO: 119; or (e) said TnsB, TnsC, and TniQ components comprise sequences having at least 80% sequence identity to SEQ ID NOs: 101-103 or variants thereof. In some embodiments: (a) said class II, type V Cas effector comprises a sequence having at least 80% sequence identity to SEQ ID NO:147 or a variant thereof; (b) said left-hand recombinase sequence comprises a sequence having at least 80% sequence identity to SEQ ID NO: 153 or a variant thereof; (c) said right-hand recombinase sequence comprises a sequence having at least 880% sequence identity to SEQ ID NO:154 or a variant thereof; (d) said engineered guide polynucleotide: (i) comprises a sequence having at least 80% sequence identity to at least about 46-60 nucleotides of any one of SEQ ID NOs: 151, 181, or 234; or (ii) comprises a sequence having at least 80% sequence identity to non-degenerate nucleotides of SEQ ID NO: 152 or 254; or (e) said TnsB, TnsC, and TniQ components comprise sequences having at least 80% sequence identity to SEQ ID NOs: 148-150 or variants thereof. In some embodiments: (a) said class II, type V Cas effector comprises a sequence having at least 80% sequence identity to SEQ ID NO:34 or a variant thereof; (b) said left-hand recombinase sequence comprises a sequence having at least 80% sequence identity to SEQ ID NO: 129 or a variant thereof; (c) said right-hand recombinase sequence comprises a sequence having at least 880% sequence identity to SEQ ID NO: 130 or a variant thereof; (d) said engineered guide polynucleotide: (i) comprises a sequence having at least 80% sequence identity to at least about 46-60 nucleotides of any one of SEQ ID NOs: 93, 157, or 210; or (ii) comprises a sequence having at least 80% sequence identity to non-degenerate nucleotides of any one of SEQ ID NOs: 97, 114, or 204, or (e) said TnsB, TnsC, and TniQ components comprise sequences having at least 80% sequence identity to SEQ ID NOs: 148-150 or variants thereof. In some embodiments: (a) said class II, type V Cas effector comprises a sequence having at least 80% sequence identity to SEQ ID NO:30 or a variant thereof; (b) said left-hand recombinase sequence comprises a sequence having at least 80% sequence identity to SEQ ID NO: 123 or a variant thereof; (c) said right-hand recombinase sequence comprises a sequence having at least 80% identity to SEQ ID NO: 124, or a variant thereof; (d) said engineered guide polynucleotide: (i) comprises a sequence having at least 80% sequence identity to at least about 46-80 nucleotides of SEQ ID NO:92; or (ii) comprises a sequence having at least 80% identity to the non-degenerate nucleotides of SEQ ID NO: 111 or 201; (e) said TnsB, TnsC, and TniQ components comprise polypeptides having a sequence having at least 80% identity to SEQ ID NOs: 31, 32, and 33, or variants thereof; or (f) said PAM sequence comprises 5′-nGTn-3′ or 5′-nGTt-3′.

In some aspects, the present disclosure provides for a system for transposing a cargo nucleotide sequence to a target nucleic acid site comprising: a first double-stranded nucleic acid comprising a cargo nucleotide sequence configured to interact with a Tn7 type transposase complex; a Cas effector complex comprising a class II, type V Cas effector and an engineered guide polynucleotide configured to hybridize to said target nucleotide sequence; and a Tn7 type transposase complex configured to bind said Cas effector complex, wherein said Tn7 type transposase complex comprises TnsB and TnsC components but does not comprise a TnsA and/or TniQ component. In some embodiments, said transposase complex binds non-covalently to said Cas effector complex. In some embodiments, said transposase complex is covalently linked to said Cas effector complex. In some embodiments, said transposase complex is fused to said Cas effector complex in a single polypeptide. In some embodiments, said Tn7 type transposase complex comprises a polypeptide having a sequencing having at least 80% sequence identity to any one of SEQ ID NOs: 39-40 or 109-110. In some embodiments, said TnsB component comprises a polypeptide comprising a sequence having at least 80% sequence identity to SEQ ID NOs: 40 or 109. In some embodiments, said TnsC component comprises a polypeptide comprising a sequence having at least 80% sequence identity to SEQ ID NOs: 39 or 110. In some embodiments, said class II, type V Cas effector is a Cas12k effector. In some embodiments, said class II, type V Cas effector comprises a sequence having at least 80% sequence identity to SEQ ID NO: 38 or SEQ ID NO:108. In some embodiments, said cargo nucleotide sequence is flanked by a left-hand transposase recognition sequence and a right-hand transposase recognition sequence. In some embodiments, the system further comprises a second double-stranded nucleic acid comprising said target nucleic acid site. In some embodiments, said double-stranded nucleic acid comprising said target nucleic acid site or said system is inside a cell. In some embodiments, the system further comprises a PAM sequence compatible with said Cas effector complex adjacent to said target nucleic acid site. In some embodiments, said PAM sequence is located 5′ of said target nucleic acid site. In some embodiments, said engineered guide polynucleotide is configured to bind said class II, type V Cas effector. In some embodiments, said TnsB and TnsC components comprise polypeptides having a sequence having at least 80% identity to SEQ ID NOs: 40 and 39 or 109 and 110, respectively. In some embodiments, said engineered guide polynucleotide comprises a sequence comprising at least about 46-80 consecutive nucleotides having at least 80% identity to any one of SEQ ID NOs: 118, 182, 183, 235, or 236, or a variant thereof. In some embodiments, said engineered guide polynucleotide comprises a sequence having at least 80% identity to non-degenerate nucleotides any one of SEQ ID NOs: 115, 116, 205, 206, 261, 235, 260, or 236, or a variant thereof. In some embodiments, said left-hand recombinase sequence comprises a sequence having at least 80% identity to SEQ ID NO:134. In some embodiments, said right-hand recombinase sequence comprises a sequence having at least 80% identity to SEQ ID NOs: 135, or a variant thereof. In some embodiments, said class II, type V Cas effector and said Tn7 type transposase complex are encoded by polynucleotide sequences comprising fewer than about 10 kilobases. In some embodiments (a) said class II, type V Cas effector comprises a sequence having at least 80% sequence identity to SEQ ID NO:38 or a variant thereof; (b) said left-hand recombinase sequence comprises a sequence having at least 80% sequence identity to SEQ ID NO: 134 or a variant thereof; (c) said right-hand recombinase sequence comprises a sequence having at least 80% identity to SEQ ID NO: 135, or a variant thereof; (d) said engineered guide polynucleotide: (i) comprises a sequence having at least 80% sequence identity to at least about 46-80 nucleotides of SEQ ID NO:182 or 235; or (ii) comprises a sequence having at least 80% identity to the non-degenerate nucleotides of SEQ ID NO:98, 115, 116, 205, or 206; or (e) said TnsB and TnsC components comprise polypeptides having a sequence having at least 80% identity to SEQ ID NOs: 40 and 39, or variants thereof.

In some aspects, the present disclosure provides for an engineered nuclease system comprising: an endonuclease comprising a RuvC domain and an HNH domain, wherein said endonuclease is derived from an uncultivated microorganism, wherein said endonuclease is a Class II, type II endonuclease comprising a sequence having at least 80% identity to SEQ ID NO: 1 or a variant thereof; and an engineered guide polynucleotide, wherein said engineered guide polynucleotide is configured to form a complex with said endonuclease and said engineered guide polynucleotide comprises a spacer sequence configured to hybridize to a target nucleic acid sequence. In some embodiments, said engineered guide polynucleotide comprises at least 60-80 consecutive nucleotides having at least 80% identity to SEQ ID NO: 12 or a variant thereof. In some embodiments, said engineered guide polynucleotide comprises a sequence having at least 80% identity to SEQ ID NO: 11 or a variant thereof.

In some aspects, the present disclosure provides for an engineered nuclease system comprising: an endonuclease comprising a RuvC domain, wherein said endonuclease is derived from an uncultivated microorganism, and wherein said endonuclease is a Class II, type V endonuclease having at least 80% identity to SEQ ID NO: 5; and an engineered guide polynucleotide, wherein said engineered guide polynucleotide is configured to form a complex with said endonuclease and said engineered guide RNA comprises a spacer sequence configured to hybridize to a target nucleic acid sequence. In some embodiments, said engineered guide polynucleotide comprises a sequence comprising at least about 46-80 consecutive nucleotides having at least 80% identity to SEQ ID NOs: 13-16, or a variant thereof.

In some aspects, the present disclosure provides for an engineered nuclease system comprising: an endonuclease comprising a RuvC domain, wherein said endonuclease is derived from an uncultivated microorganism, and wherein said endonuclease is a Class II, type V-K endonuclease having at least 80% identity to any one of SEQ ID NOs:22, 26, 30, 34, 55-89, 104, or 147, or a variant thereof; and an engineered guide polynucleotide, wherein said engineered guide polynucleotide is configured to form a complex with said endonuclease and said engineered guide RNA comprises a spacer sequence configured to hybridize to a target nucleic acid sequence. In some embodiments, said engineered guide polynucleotide comprises a sequence comprising at least about 46-80 consecutive nucleotides having at least 80% identity to any one of SEQ ID NOs: 90, 91, 92, 93, 117, 151, 156-181, or 209-234, or a variant thereof. In some embodiments, said engineered guide polynucleotide comprises a sequence having at least 80% sequence identity to non-degenerate nucleotides of any one of SEQ ID NOs: 111-114 or 201-206, 255, 262, 256, 209, 257, 263, 258, 210, or a variant thereof.

In some aspects, the present disclosure provides for an engineered nuclease system comprising: an endonuclease comprising a RuvC domain, wherein said endonuclease is derived from an uncultivated microorganism, and wherein said endonuclease is a Class II, type V-K endonuclease having at least 80% identity to any one of SEQ ID NO: 38 or SEQ ID NO: 108, or a variant thereof; and an engineered guide polynucleotide, wherein said engineered guide polynucleotide is configured to form a complex with said endonuclease and said engineered guide RNA comprises a spacer sequence configured to hybridize to a target nucleic acid sequence. In some embodiments, said engineered guide polynucleotide comprises a sequence comprising at least about 46-80 consecutive nucleotides having at least 80% identity to any one of SEQ ID NOs: 118, 182, 183, 235, or 236, or a variant thereof. In some embodiments, said engineered guide polynucleotide comprises a sequence having at least 80% identity to non-degenerate nucleotides of any one of SEQ ID NOs: 111-114 or 201-206, 255, 262, 256, 209, 257, 263, 258, 210, 115, 116, 205, 206, 261, 235, 260, or 236, or a variant thereof.

In some aspects, the present disclosure provides for an engineered nuclease system comprising: a Class I, type I-F Cas endonuclease comprising at least one Cas6, Cas7, or Cas8 polypeptide comprising a sequence having at least 80% identity to any one of SEQ ID NO: 41-43, or 48-50, or a variant thereof; and an engineered guide RNA, wherein said engineered guide RNA is configured to form a complex with said endonuclease and said engineered guide RNA comprises a spacer sequence configured to hybridize to a target nucleic acid sequence. In some embodiments, said engineered guide polynucleotide comprises a sequence having at least 80% identity to non-degenerate nucleotides of any one of SEQ ID NOs: 121, 122, 207, or 208.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 depicts typical organizations of CRISPR/Cas loci of different classes and types.

FIG. 2 depicts the architecture of a natural Class II Type II crRNA/tracrRNA pair, compared to a hybrid sgRNA wherein the crRNA and tracrRNA are joined.

FIG. 3 depicts the two pathways found in Tn7 and Tn7-like elements.

FIGS. 4A-4C depict the genomic context of a Type II Tn7 reduced CAST of the family MG36. FIG. 4A shows the MG36-5 CAST system consists of a CRISPR array (CRISPR repeats), a Type II nuclease with RuvC and HNH endonuclease domains, and four predicted transposase protein open reading frames. The catalytic transposase TnsB is encoded as two subunits. FIG. 4B shows two transposon ends are predicted for the MG36-1 CAST system (TIR-1 and TIR-2). FIG. 4C shows alignment of the predicted Type II Tn7 reduced CAST transposon left end (LE) and right end (RE) sequences, with annotated repeats as arrows. The left and right ends were labeled by their orientation.

FIGS. 5A and 5B depict the genomic context of a Type V Tn7 CAST of the family MG39. FIG. 5A shows the MG39-1 CAST system consists of a Type V nuclease, four predicted transposon proteins (TnsABC and TniQ), and a CRISPR array. The transposon ends were predicted for the MG39-1 CAST system (TIR-1). FIG. 5B shows the alignment of the predicted Type V Tn7 CAST transposon left end (LE) and right end (RE) sequences, with annotated inverted repeats represented as arrows.

FIG. 6 and FIG. 7 depict predicted structures (predicted, for example in Example 3) of corresponding sgRNAs of CAST systems described herein.

FIG. 8 depicts the genomic context of MG108-1, a system described herein. This candidate is a Cas12K CAST which naturally lacks TniQ. Genes in the genomic fragment are represented by arrows.

FIG. 9 depicts the phylogenetic gene tree of Cas12k effector sequences. The tree was inferred from a multiple sequence alignment of 64 Cas12k sequences recovered here (orange and black branches) and 229 reference Cas12k sequences from public databases (grey branches). Orange branches indicate Cas12k effectors with confirmed association with CAST transposon components.

FIGS. 10A-10C depict MG110 Cascade CAST. FIG. 10A provides genomic context of the MG110-1 Cascade CAST. Full Tn7 suite (TnsA, TnsB, TnsC/TniB, TniQ) and defective Cascade suite (Cas6, Cas7, fused Cas5-Cas8) are represented by orange arrows. TIR flanking the CAST transposon are represented by connected arrows. FIG. 10B provides a repeat secondary structure indicates a stem-loop structure of the crRNA. FIG. 10C provides a sequence alignment of CRISPR repeats from A, wodanis, V. cholerae, and the MG110 family CASTs indicates conserved motifs indicative of the crRNA stem-loop secondary structure.

FIG. 11A depicts the MG64-3 CRISPR locus. The tracrRNA is encoded upstream from the CRISPR array, while the transposon end is encoded downstream (inner black box). A sequence corresponding to a partial 3′ CRISPR repeat and a partial spacer are encoded within the transposon (outer box). The self-matching spacer is encoded outside of the transposon end. FIG. 11B depicts tracrRNA sequence alignment for various CASTs provided herein. Alignment of tracrRNA sequences shows regions of conservation. In particular, the sequence “TGCTTTC” at sequence position 92-98 (top box) is suggested to be important for sgRNA tertiary structure and for a non-continuous repeat-anti-repeat pairing with the crRNA. We also suggest that the hairpin “CYCC(n6)GGRG” at positions 265-278 (bottom box) is important for function, possibly positioning the downstream sequence for crRNA pairing. FIG. 11C shows presence of other important repeat-anti-repeat (RAR) motifs in e.g. MG64-2, MG64-4, MG64-5, MG64-6, MG64-7, and MG108-1 families.

FIG. 12A depicts the predicted structure of MG64-2 sgRNA. FIG. 12B depicts the predicted structure of MG64-4 sgRNA. FIG. 12C depicts the predicted structure of MG64-6 sgRNA. FIG. 12D depicts the predicted structure of MG64-7 sgRNA. FIG. 12E depicts the predicted structure of MG108-1 sgRNA.

FIGS. 13A-13C depict PCR, PAM, and Sanger sequencing data which demonstrate that MG64-6 is active in vitro. Using the protocol described for In vitro targeted integrase activity, the effector protein and its TnsB, TnsC, and TniQ proteins were expressed in an in vitro transcription/translation system. After translation, the target DNA, cargo DNA, and sgRNA were added in reaction buffer. Integration was assayed by PCR across the target/donor junctions. FIG. 13A depicts a gel image of PCRs of transposition showing apo (no sgRNA) and 64-6 with sgRNA 64-6 sgRNA. The PCR 3 detects the RE junction, PAM distal. PCR 4 is LE junction, PAM distal. PCR 5 is RE junction, PAM proximal. PCR 6 is LE junction, PAM proximal. The PCRs are paired across the different possible orientations (PCR 3 and 6 vs PCR 4 and 5). The LE-PAM proximal and RE-PAM distal orientation is preferred. FIG. 13B depicts PAMs from the in vitro transposition assay, sequencing PCRs 5 and 6. FIG. 13C depicts Sanger data which shows the junction of transposition where the excision occurs in the donor DNA. The first panel shows PCRs 3 and 5 (the RE). The second panel shows PCR 4 and 6 (the LE). The Sanger sequencing reaction is of the donor-target product, so the point where the sequencing stops matching the donor DNA is when junction occurs (dark bars underneath sequencing peaks)

FIG. 14 depicts next-generation sequencing (NGS) results of the in vitro transposition products which reveal the insertion site preferences. The NGS reads were processed in CRISPResso2 compared to a reference sequence with transposition at position 60. Indels from this correspond to transpositions earlier or later than this arbitrary reference sequence.

FIG. 15 depicts electrophoretic mobility shift assay (EMSA) results of the 64-2 TnsB and its RE DNA sequence. The EMSA results confirm binding and TnsB recognition. The TnsB protein was expressed in an in vitro transcription/translation system, incubated with FAM-labeled DNA containing the RE sequence, and then separated on a native 5% TBE gel. Binding is observed as a shift upwards in the labeled band. Multiple TnsB binding sites leads to multiple shifts in the EMSA. Lane 1: FAM-labeled DNA only. Lane 2: FAM DNA plus the in vitro transcription/translation system (no TnsB protein). Lane 3: FAM DNA plus TnsB. Upshift of the labeled band in Lane 3 indicates binding of the RE sequence by TnsB, indicating it contains an active RE transposition sequence.

### BRIEF DESCRIPTION OF THE SEQUENCE LISTING

The Sequence Listing filed herewith provides exemplary polynucleotide and polypeptide sequences for use in methods, compositions, and systems according to the disclosure. Below are exemplary descriptions of sequences therein.

### MG36

SEQ ID NO: 1 shows a full-length peptide sequence of a MG36 Cas effector.

SEQ ID NOs: 2-5 show peptide sequences of MG36 transposition proteins that may comprise a recombinase or transposase complex associated with a MG36 Cas effector. The addition of −B1, −B2, −T1, and −C to the end of the labels denotes similarity to TnsB1, TnsB2, TnsT1, and TniC proteins of Tn7-like systems, respectively.

SEQ ID NO: 11 shows a nucleotide sequence of an sgRNA engineered to function with an MG36 Cas effector.

SEQ ID NO: 12 shows a nucleotide sequence of a MG36 tracrRNAs derived from the same loci as a MG36 Cas effector.

SEQ ID NOs: 17-18 show nucleotide sequences of left-hand transposase recognition sequences associated with a MG36 system.

SEQ ID NO: 19 shows a nucleotide sequence of a right-hand transposase recognition sequence associated with a MG36 system.

### MG39

SEQ ID NO: 6 shows the full-length peptide sequence of a MG39-1 Cas effector.

SEQ ID Nos: 7-10 show the peptide sequences of MG39-1 transposition proteins that may comprise a recombinase or transposase complex associated with the MG39-1 Cas effector.

SEQ ID NOs: 13-16 show nucleotide sequences of MG39 tracrRNAs derived from the same loci as a MG39 Cas effector.

SEQ ID NO: 20 shows a nucleotide sequence of a left-hand transposase recognition sequence associated with a MG39 system.

SEQ ID NO: 21 shows a nucleotide sequence of a right-hand transposase recognition sequence associated with a MG39 system.

MG64

SEQ ID NOs: 22, 26, 30, 34, 55-89, 104, and 147 show the full-length peptide sequences of MG64 Cas effectors.

SEQ ID NOs: 23-25, 27-29, 31-33, 35-37, 101-103, 105-107, and 148-150 show the peptide sequences of MG64 transposition proteins that may comprise a recombinase or transposase complex associated with MG64 Cas effectors. The addition of −A, −B, −C, and −Q to the end of the labels denotes similarity to TnsA, TnsB, TnsC, and TniQ proteins of Tn7-like systems, respectively.

SEQ ID NOs: 90-93, 117, 151, 156-181, and 209-234 show nucleotide sequences of MG64 tracrRNAs derived from the same loci as a MG64 effector.

SEQ ID NOs: 94-97, 119, 152, and 184-200 show nucleotide sequences of MG64 target CRISPR repeats.

SEQ ID NOs: 237-259 show nucleotide sequences of MG64 crRNAs.

Seq ID NOs: 111-114 and 201-204 show nucleotide sequences of single guide RNAs engineered to function with MG64 Cas effectors.

SEQ ID NOs: 123, 125, 127, 129, 131, 133, and 153 show nucleotide sequences of left-hand transposase recognition sequences associated with a MG64 system.

SEQ ID NOs: 124, 126, 128, 130, 132, 154, and 155 show nucleotide sequences of right-hand transposase recognition sequences associated with a MG64 system.

### MG108

SEQ ID NOs: 38, and 108 show the full-length peptide sequences of MG108 Cas effectors.

SEQ ID NOs: 39-40 and 109-110 show the peptide sequences of MG108 transposition proteins that may comprise a recombinase or transposase complex associated with MG108 Cas effectors. The addition of −A, −B, −C, and −Q to the end of the labels denotes similarity to TnsA, TnsB, TnsC, and TniQ proteins of Tn7-like systems, respectively.

SEQ ID NO: 98 and 120 show nucleotide sequences of MG108 target CRISPR repeats.

SEQ ID NO: 260-261 show nucleotide sequences of MG108 crRNAs.

Seq ID NOs: 115-116 and 205-206 show nucleotide sequences of single guide RNAs engineered to function with MG108 Cas effectors.

SEQ ID NOs: 118, 182-183, and 235-236 show nucleotide sequences of MG108 tracrRNAs derived from the same loci as a MG108 effector.

SEQ ID NO: 134 shows a nucleotide sequence of a left-hand transposase recognition sequence associated with a MG108 system.

SEQ ID NO: 135 shows a nucleotide sequence of a right-hand transposase recognition sequence associated with a MG108 system.

MG110

SEQ ID NOs: 41-43 and 48-50 show the full-length peptide sequences of MG110 Cas effectors. The addition of −6, −7, and −8 to the end of the labels denotes similarity to cas6, cas7, and cas8 proteins of class I, type I-F systems, respectively.

SEQ ID NOs: 44-47 and 51-54 show the peptide sequences of MG110 transposition proteins that may comprise a recombinase or transposase complex associated with MG110 Cas effectors. The addition of −A, −B, −C, and −Q to the end of the labels denotes similarity to TnsA, TnsB, TnsC, and TniQ proteins of Tn7-like systems, respectively.

SEQ ID NOs: 99-100 show nucleotide sequences of MG110 target CRISPR repeats.

SEQ ID NOs: 121-122 and 207-208 show nucleotide sequences of MG110 crRNAs.

SEQ ID NOs: 136 and 138 show nucleotide sequences of left-hand transposase recognition sequences associated with a MG110 system.

SEQ ID NOs: 137 and 139 show nucleotide sequences of right-hand transposase recognition sequences associated with a MG110 system.

### Other Sequences

SEQ ID NOs: 140-141 show peptide sequences of nuclear localizing signals.

SEQ ID NOs: 142-143 show peptide sequences of linkers.

SEQ ID NOs: 144-146 show peptide sequences of epitope tags.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The practice of some methods disclosed herein employ, unless otherwise indicated, techniques of immunology, biochemistry, chemistry, molecular biology, microbiology, cell biology, genomics, and recombinant DNA. See for example Sambrook and Green, Molecular Cloning: A Laboratory Manual, 4th Edition (2012); the series Current Protocols in Molecular Biology (F. M. Ausubel, et al, eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)) (which is entirely incorporated by reference herein).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 20%, up to 15%, up to 10%, up to 5%, or up to 1% of a given value.

As used herein, a “cell” generally refers to a biological cell. A cell may be the basic structural, functional and/or biological unit of a living organism. A cell may originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g., kelp), a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g., a cell can be a synthetically made, sometimes termed an artificial cell).

The term “nucleotide,” as used herein, generally refers to a base-sugar-phosphate combination. A nucleotide may comprise a synthetic nucleotide. A nucleotide may comprise a synthetic nucleotide analog. Nucleotides may be monomeric units of a nucleic acid sequence (e.g., deoxyribonucleic acid (DNA) and ribonucleic acid (RNA)). The term nucleotide may include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives may include, for example, [aS]dATP, 7-deaza-dGTP and 7-deaza-dATP, and nucleotide derivatives that confer nuclease resistance on the nucleic acid molecule containing them. The term nucleotide as used herein may refer to dideoxyribonucleoside triphosphates (ddNTPs) and their derivatives. Illustrative examples of dideoxyribonucleoside triphosphates may include, but are not limited to, ddATP, ddCTP, ddGTP, ddITP, and ddTTP. A nucleotide may be unlabeled or detectably labeled, such as using moieties comprising optically detectable moieties (e.g., fluorophores). Labeling may also be carried out with quantum dots. Detectable labels may include, for example, radioactive isotopes, fluorescent labels, chemiluminescent labels, bioluminescent labels, and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif; FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g., biotin-11-dUTP, biotin-16-dUTP, biotin-20-dUTP).

The terms “polynucleotide,” “oligonucleotide,” and “nucleic acid” are used interchangeably to generally refer to a polymeric form of nucleotides of any length, either deoxyribonucleotides or ribonucleotides, or analogs thereof, either in single-, double-, or multi-stranded form. A polynucleotide may be exogenous or endogenous to a cell. A polynucleotide may exist in a cell-free environment. A polynucleotide may be a gene or fragment thereof. A polynucleotide may be DNA. A polynucleotide may be RNA. A polynucleotide may have any three-dimensional structure and may perform any function. A polynucleotide may comprise one or more analogs (e.g., altered backbone, sugar, or nucleobase). If present, modifications to the nucleotide structure may be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides may be interrupted by non-nucleotide components.

The terms “transfection” or “transfected” generally refer to introduction of a nucleic acid into a cell by non-viral or viral-based methods. The nucleic acid molecules may be gene sequences encoding complete proteins or functional portions thereof. See, e.g., Sambrook et al., 1989, Molecular Cloning: A Laboratory Manual, 18.1-18.88.

The terms “peptide,” “polypeptide,” and “protein” are used interchangeably herein to generally refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer may be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids may include natural amino acids and non-natural amino acids, which have been chemically modified to include a group or a chemical moiety not naturally present on the amino acid. Amino acid analogues may refer to amino acid derivatives. The term “amino acid” includes both D-amino acids and L-amino acids.

As used herein, the “non-native” can generally refer to a nucleic acid or polypeptide sequence that is not found in a native nucleic acid or protein. Non-native may refer to affinity tags. Non-native may refer to fusions. Non-native may refer to a naturally occurring nucleic acid or polypeptide sequence that comprises mutations, insertions and/or deletions. A non-native sequence may exhibit and/or encode for an activity (e.g., enzymatic activity, methyltransferase activity, acetyltransferase activity, kinase activity, ubiquitinating activity, etc.) that may also be exhibited by the nucleic acid and/or polypeptide sequence to which the non-native sequence is fused. A non-native nucleic acid or polypeptide sequence may be linked to a naturally-occurring nucleic acid or polypeptide sequence (or a variant thereof) by genetic engineering to generate a chimeric nucleic acid and/or polypeptide sequence encoding a chimeric nucleic acid and/or polypeptide.

The term “promoter”, as used herein, generally refers to the regulatory DNA region which controls transcription or expression of a gene and which may be located adjacent to or overlapping a nucleotide or region of nucleotides at which RNA transcription is initiated. A promoter may contain specific DNA sequences which bind protein factors, often referred to as transcription factors, which facilitate binding of RNA polymerase to the DNA leading to gene transcription. A ‘basal promoter’, also referred to as a ‘core promoter’, may generally refer to a promoter that contains all the basic necessary elements to promote transcriptional expression of an operably linked polynucleotide. Eukaryotic basal promoters typically, though not necessarily, contain a TATA-box and/or a CAAT box.

The term “expression”, as used herein, generally refers to the process by which a nucleic acid sequence or a polynucleotide is transcribed from a DNA template (such as into mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, “operably linked”, “operable linkage”, “operatively linked”, or grammatical equivalents thereof generally refer to juxtaposition of genetic elements, e.g., a promoter, an enhancer, a polyadenylation sequence, etc., wherein the elements are in a relationship permitting them to operate in the expected manner. For instance, a regulatory element, which may comprise promoter and/or enhancer sequences, is operatively linked to a coding region if the regulatory element helps initiate transcription of the coding sequence. There may be intervening residues between the regulatory element and coding region so long as this functional relationship is maintained.

A “vector” as used herein, generally refers to a macromolecule or association of macromolecules that comprises or associates with a polynucleotide and which may be used to mediate delivery of the polynucleotide to a cell. Examples of vectors include plasmids, viral vectors, liposomes, and other gene delivery vehicles. The vector generally comprises genetic elements, e.g., regulatory elements, operatively linked to a gene to facilitate expression of the gene in a target.

As used herein, “an expression cassette” and “a nucleic acid cassette” are used interchangeably generally to refer to a combination of nucleic acid sequences or elements that are expressed together or are operably linked for expression. In some cases, an expression cassette refers to the combination of regulatory elements and a gene or genes to which they are operably linked for expression.

A “functional fragment” of a DNA or protein sequence generally refers to a fragment that retains a biological activity (either functional or structural) that is substantially similar to a biological activity of the full-length DNA or protein sequence. A biological activity of a DNA sequence may be its ability to influence expression in a manner known to be attributed to the full-length sequence.

As used herein, an “engineered” object generally indicates that the object has been modified by human intervention. According to non-limiting examples: a nucleic acid may be modified by changing its sequence to a sequence that does not occur in nature; a nucleic acid may be modified by ligating it to a nucleic acid that it does not associate with in nature such that the ligated product possesses a function not present in the original nucleic acid; an engineered nucleic acid may synthesized in vitro with a sequence that does not exist in nature; a protein may be modified by changing its amino acid sequence to a sequence that does not exist in nature; an engineered protein may acquire a new function or property. An “engineered” system comprises at least one engineered component.

As used herein, “synthetic” and “artificial” are used interchangeably to refer to a protein or a domain thereof that has low sequence identity (e.g., less than 50% sequence identity, less than 25% sequence identity, less than 10% sequence identity, less than 5% sequence identity, less than 1% sequence identity) to a naturally occurring human protein. For example, VPR and VP64 domains are synthetic transactivation domains.

The term “tracrRNA” or “tracr sequence”, as used herein, can generally refer to a nucleic acid with at least about 5%, 10%, 20%, 30%, 40%, 50%, 60%, 70%, 80%, 90%, 95%, or 100% sequence identity and/or sequence similarity to a wild type exemplary tracrRNA sequence (e.g., a tracrRNA from S. pyogenes S, aureus, etc. or SEQ ID NOs: *_*), tracrRNA can refer to a nucleic acid with at most about 5%, 10%, 20%, 30%, 40%, 50%, 60%, 70%, 80%, 90%, or 100% sequence identity and/or sequence similarity to a wild type exemplary tracrRNA sequence (e.g., a tracrRNA from S. pyogenes S, aureus, etc), tracrRNA may refer to a modified form of a tracrRNA that can comprise a nucleotide change such as a deletion, insertion, or substitution, variant, mutation, or chimera. A tracrRNA may refer to a nucleic acid that can be at least about 60% identical to a wild type exemplary tracrRNA (e.g., a tracrRNA from S. pyogenes S, aureus, etc) sequence over a stretch of at least 6 contiguous nucleotides. For example, a tracrRNA sequence can be at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical to a wild type exemplary tracrRNA (e.g., a tracrRNA from S. pyogenes S, aureus, etc) sequence over a stretch of at least 6 contiguous nucleotides. Type II tracrRNA sequences can be predicted on a genome sequence by identifying regions with complementarity to part of the repeat sequence in an adjacent CRISPR array.

As used herein, a “guide nucleic acid” can generally refer to a nucleic acid that may hybridize to another nucleic acid. A guide nucleic acid may be RNA. A guide nucleic acid may be DNA. The guide nucleic acid may be programmed to bind to a sequence of nucleic acid site-specifically. The nucleic acid to be targeted, or the target nucleic acid, may comprise nucleotides. The guide nucleic acid may comprise nucleotides. A portion of the target nucleic acid may be complementary to a portion of the guide nucleic acid. The strand of a double-stranded target polynucleotide that is complementary to and hybridizes with the guide nucleic acid may be called the complementary strand. The strand of the double-stranded target polynucleotide that is complementary to the complementary strand, and therefore may not be complementary to the guide nucleic acid may be called noncomplementary strand. A guide nucleic acid may comprise a polynucleotide chain and can be called a “single guide nucleic acid.” A guide nucleic acid may comprise two polynucleotide chains and may be called a “double guide nucleic acid.” If not otherwise specified, the term “guide nucleic acid” may be inclusive, referring to both single guide nucleic acids and double guide nucleic acids. A guide nucleic acid may comprise a segment that can be referred to as a “nucleic acid-targeting segment” or a “nucleic acid-targeting sequence.” A nucleic acid-targeting segment may comprise a sub-segment that may be referred to as a “protein binding segment” or “protein binding sequence” or “Cas protein binding segment”.

The term “sequence identity” or “percent identity” in the context of two or more nucleic acids or polypeptide sequences, generally refers to two (e.g., in a pairwise alignment) or more (e.g., in a multiple sequence alignment) sequences that are the same or have a specified percentage of amino acid residues or nucleotides that are the same, when compared and aligned for maximum correspondence over a local or global comparison window, as measured using a sequence comparison algorithm. Suitable sequence comparison algorithms for polypeptide sequences include, e.g., BLASTP using parameters of a wordlength (W) of 3, an expectation (E) of 10, and the BLOSUM62 scoring matrix setting gap costs at existence of 11, extension of 1, and using a conditional compositional score matrix adjustment for polypeptide sequences longer than 30 residues; BLASTP using parameters of a wordlength (W) of 2, an expectation (E) of 1000000, and the PAM30 scoring matrix setting gap costs at 9 to open gaps and 1 to extend gaps for sequences of less than 30 residues (these are the default parameters for BLASTP in the BLAST suite available at https://blast.ncbi.nlm.nih.gov); CLUSTALW with parameters of; the Smith-Waterman homology search algorithm with parameters of a match of 2, a mismatch of −1, and a gap of −1; MUSCLE with default parameters; MAFFT with parameters retree of 2 and maxiterations of 1000; Novafold with default parameters; HMMER hmmalign with default parameters.

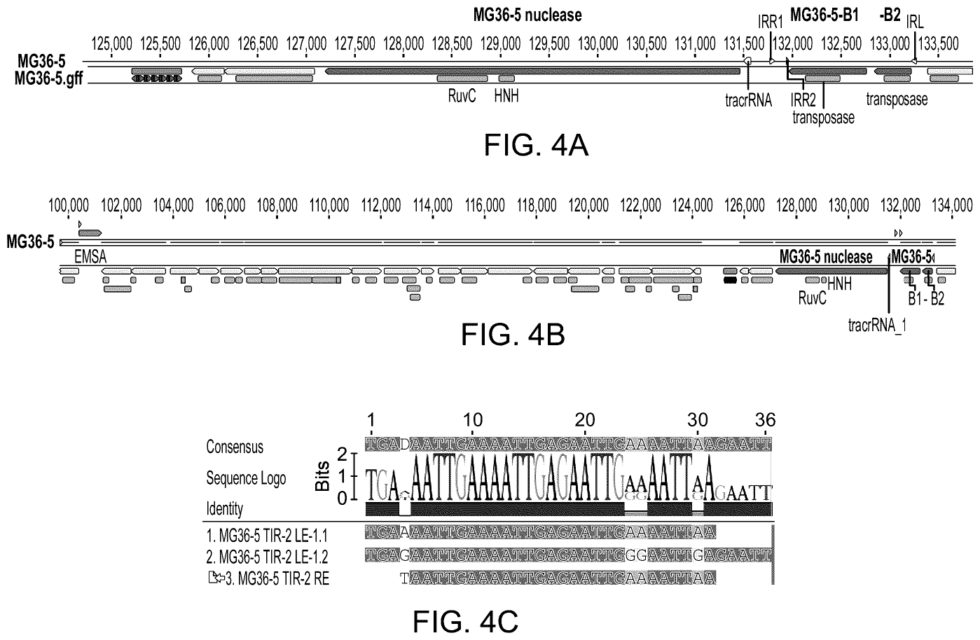

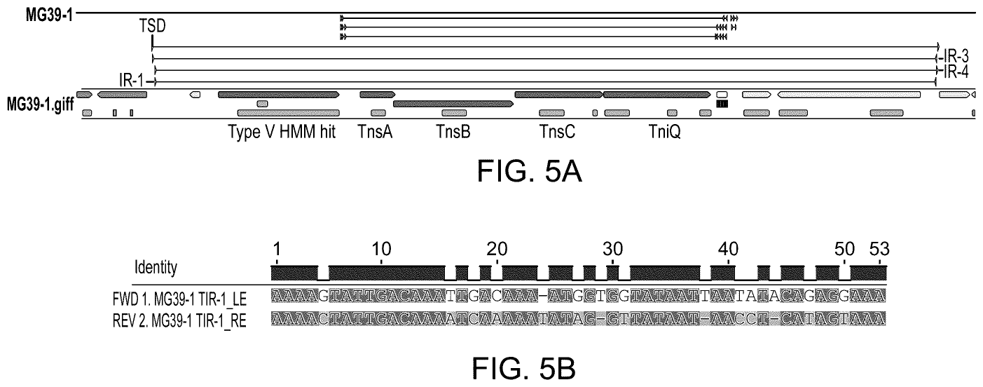

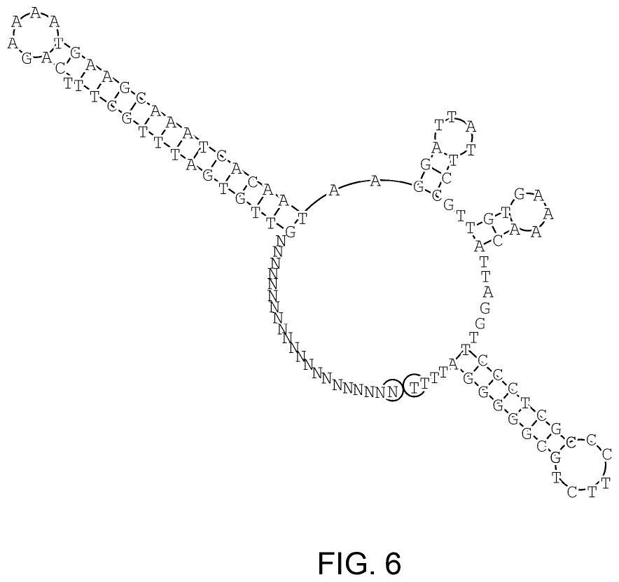

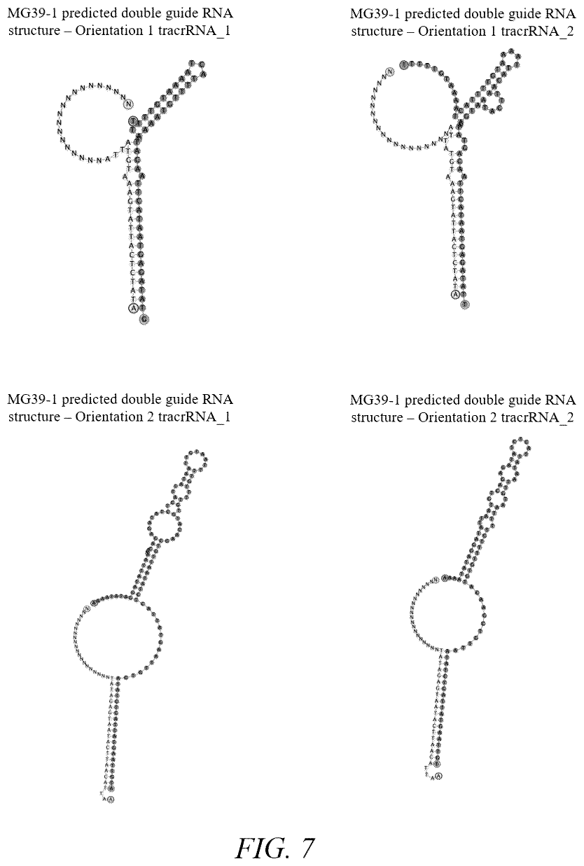

Included in the current disclosure are variants of any of the enzymes described herein with one or more conservative amino acid substitutions. Such conservative substitutions can be made in the amino acid sequence of a polypeptide without disrupting the three-dimensional structure or function of the polypeptide. Conservative substitutions can be accomplished by substituting amino acids with similar hydrophobicity, polarity, and R chain length for one another. Additionally, or alternatively, by comparing aligned sequences of homologous proteins from different species, conservative substitutions can be identified by locating amino acid residues that have been mutated between species (e.g. non-conserved residues without altering the basic functions of the encoded proteins. Such conservatively substituted variants may include variants with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity any one of the systems described herein (e.g., MG36 or MG39 systems described herein). In some embodiments, such conservatively substituted variants are functional variants. Such functional variants can encompass sequences with substitutions such that the activity of critical active site residues of the endonuclease is not disrupted. In some embodiments, a functional variant of any of the systems described herein lack substitution of at least one of the conserved or functional residues called out in FIGS. 4 and 5. In some embodiments, a functional variant of any of the systems described herein lacks substitution of all of the conserved or functional residues called out in FIGS. 4 and 5.

Conservative substitution tables providing functionally similar amino acids are available from a variety of references (see, for example, Creighton, Proteins: Structures and Molecular Properties (W H Freeman & Co.; 2^{nd} Edition (December 1993))). The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

As used herein, the term “RuvC_III domain” generally refers to a third discontinuous segment of a RuvC endonuclease domain (the RuvC nuclease domain being comprised of three discontiguous segments, RuvC_I, RuvC_II, and RuvC_III). A RuvC domain or segments thereof can generally be identified by alignment to known domain sequences, structural alignment to proteins with annotated domains, or by comparison to Hidden Markov Models (HMMs) built based on known domain sequences (e.g., Pfam HMM PF18541 for RuvC_III).

As used herein, the term “HNH domain” generally refers to an endonuclease domain having characteristic histidine and asparagine residues. An HNH domain can generally be identified by alignment to known domain sequences, structural alignment to proteins with annotated domains, or by comparison to Hidden Markov Models (HMMs) built based on known domain sequences (e.g., Pfam HMM PF01844 for domain HNH).

As used herein, the term “recombinase” generally refers to a site-specific enzyme that mediates the recombination of DNA between recombinase recognition sequences, which results in the excision, integration, inversion, or exchange (e.g., translocation) of DNA fragments between the recombinase recognition sequences.

As used herein, the term “recombine,” or “recombination,” in the context of a nucleic acid modification (e.g., a genomic modification), generally refers to the process by which two or more nucleic acid molecules, or two or more regions of a single nucleic acid molecule, are modified by the action of a recombinase protein. Recombination can result in, inter alia, the insertion, inversion, excision, or translocation of a nucleic acid sequence, e.g., in or between one or more nucleic acid molecules.

As used herein, the term “transposon” generally refers to mobile elements that move in and out of genomes carrying “cargo DNA” with them. In some cases, these transposons may differ on the type of nucleic acid to transpose, the type of repeat at the ends of the transposon, the type of cargo to be carried or by the mode of transposition (i.e. self-repair or host-repair). As used herein, the term “transposase” or “transposases” generally refers to an enzyme that binds to the end of a transposon and catalyzes its movement to another part of the genome. In some cases, the movement may be by a cut and paste mechanism or a replicative transposition

As used herein, the term “Tn7” or “Tn7-like transposase” generally refers to a family of transposases comprising three main components: a heteromeric transposase (TnsA and/or TnsB) alongside a regulator protein (TnsC). In addition to the TnsABC transposition proteins, Tn7 elements can encode dedicated target site-selection proteins, TnsD and TnsE. In conjunction with TnsABC, the sequence-specific DNA-binding protein TnsD directs transposition into a conserved site referred to as the “Tn7 attachment site,” attTn7, TnsD is a member of a large family of proteins that also includes TniQ, TniQ has been shown to target transposition into resolution sites of plasmids.

In some cases, the CAST systems described herein may comprise one or more Tn7 or Tn7 like transposases. In certain example embodiments, the Tn7 or Tn7 like transposase comprises a multimeric protein complex. In certain example embodiments, the multimeric protein complex comprises TnsA, TnsB, TnsC, or TniQ. In these combinations, the transposases (TnsA, TnsB, TnsC, TniQ) may form complexes or fusion proteins with each other.

As used herein, the term “Cas12k”(alternatively “class II, type V-K”) generally refers to a subtype of Type V CRISPR systems that have been found to be defective in nuclease activity (e.g. they may comprise at least one defective RuvC domain that lacking at least one catalytic residue important for DNA cleavage). Such subtype of effectors have been generally associated with CAST systems.

As used herein, the term “type I-F” (alternatively class I, type I-F CRISPR) generally refers to a subtype of class I, type I CRISPR systems. Such systems generally comprise multi-component CRISPR effectors comprising Cas8, Cas7, and Cas6 proteins. In some cases, such systems are found associated with CAST systems. In some cases, type I-F CRISPR systems comprise crRNAs comprising an 8-nt 5′ handles for Cas8 and/or Cas5 binding, 32-nt spacers bound by six copies of Cas7 for target recognition, or a 20-nt 3′ hairpins for Cas6 binding and pre-crRNA processing. In some cases, type-F systems utilize a 5′-CC PAM on the non-target strand for target binding.

### Overview

The discovery of new Cas enzymes with unique functionality and structure may offer the potential to further disrupt deoxyribonucleic acid (DNA) editing technologies, improving speed, specificity, functionality, and ease of use. Relative to the predicted prevalence of Clustered Regularly Interspaced Short Palindromic Repeats (CRISPR) systems in microbes and the sheer diversity of microbial species, relatively few functionally characterized CRISPR/Cas enzymes exist in the literature. This is partly because a huge number of microbial species may not be readily cultivated in laboratory conditions. Metagenomic sequencing from natural environmental niches that represent large numbers of microbial species may offer the potential to drastically increase the number of new CRISPR/Cas systems known and speed the discovery of new oligonucleotide editing functionalities. A recent example of the fruitfulness of such an approach is demonstrated by the 2016 discovery of CasX/CasY CRISPR systems from metagenomic analysis of natural microbial communities.

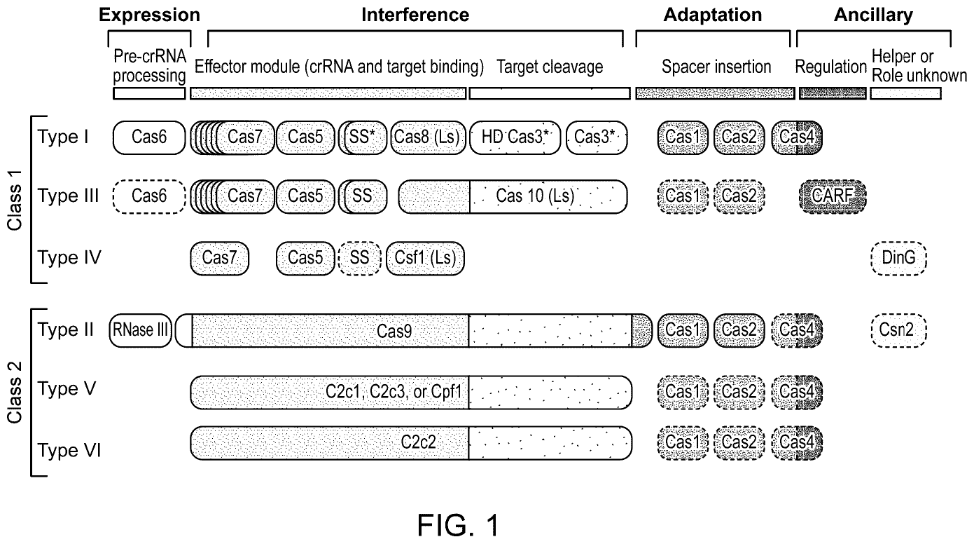

CRISPR/Cas systems are RNA-directed nuclease complexes that have been described to function as an adaptive immune system in microbes. In their natural context, CRISPR/Cas systems occur in CRISPR (clustered regularly interspaced short palindromic repeats) operons or loci, which generally comprise two parts: (i) an array of short repetitive sequences (30-40 bp) separated by equally short spacer sequences, which encode the RNA-based targeting element; and (ii) ORFs encoding the Cas encoding the nuclease polypeptide directed by the RNA-based targeting element alongside accessory proteins/enzymes. Efficient nuclease targeting of a particular target nucleic acid sequence generally requires both (i) complementary hybridization between the first 6-8 nucleic acids of the target (the target seed) and the crRNA guide; and (ii) the presence of a protospacer-adjacent motif (PAM) sequence within a defined vicinity of the target seed (the PAM usually being a sequence not commonly represented within the host genome). Depending on the exact function and organization of the system, CRISPR-Cas systems are commonly organized into 2 classes, 5 types and 16 subtypes based on shared functional characteristics and evolutionary similarity (see FIG. 1).

Class I CRISPR-Cas systems have large, multisubunit effector complexes, and comprise Types I, III, and IV.

Type I CRISPR-Cas systems are considered of moderate complexity in terms of components. In Type I CRISPR-Cas systems, the array of RNA-targeting elements is transcribed as a long precursor crRNA (pre-crRNA) that is processed at repeat elements to liberate short, mature crRNAs that direct the nuclease complex to nucleic acid targets when they are followed by a suitable short consensus sequence called a protospacer-adjacent motif (PAM). This processing occurs via an endoribonuclease subunit (Cas6) of a large endonuclease complex called Cascade, which also comprises a nuclease (Cas3) protein component of the crRNA-directed nuclease complex. Cas I nucleases function primarily as DNA nucleases.

Type III CRISPR systems may be characterized by the presence of a central nuclease, known as Cas10, alongside a repeat-associated mysterious protein (RAMP) that comprises Csm or Cmr protein subunits. Like in Type I systems, the mature crRNA is processed from a pre-crRNA using a Cas6-like enzyme. Unlike type I and II systems, type III systems appear to target and cleave DNA-RNA duplexes (such as DNA strands being used as templates for an RNA polymerase).

Type IV CRISPR-Cas systems possess an effector complex that consists of a highly reduced large subunit nuclease (csf1), two genes for RAMP proteins of the Cas5 (csf3) and Cas7 (csf2) groups, and, in some cases, a gene for a predicted small subunit; such systems are commonly found on endogenous plasmids.

Class II CRISPR-Cas systems generally have single-polypeptide multidomain nuclease effectors, and comprise Types II, V and VI.

Type II CRISPR-Cas systems are considered the simplest in terms of components. In Type II CRISPR-Cas systems, the processing of the CRISPR array into mature crRNAs does not require the presence of a special endonuclease subunit, but rather a small trans-encoded crRNA (tracrRNA) with a region complementary to the array repeat sequence; the tracrRNA interacts with both its corresponding effector nuclease (e.g. Cas9) and the repeat sequence to form a precursor dsRNA structure, which is cleaved by endogenous RNAse III to generate a mature effector enzyme loaded with both tracrRNA and crRNA. Cas II nucleases are known as DNA nucleases. Type 2 effectors generally exhibit a structure consisting of a RuvC-like endonuclease domain that adopts the RNase H fold with an unrelated HNH nuclease domain inserted within the folds of the RuvC-like nuclease domain. The RuvC-like domain is responsible for the cleavage of the target (e.g., crRNA complementary) DNA strand, while the HNH domain is responsible for cleavage of the displaced DNA strand.

Type V CRISPR-Cas systems are characterized by a nuclease effector (e.g. Cas12) structure similar to that of Type II effectors, comprising a RuvC-like domain. Similar to Type II, most (but not all) Type V CRISPR systems use a tracrRNA to process pre-crRNAs into mature crRNAs; however, unlike Type II systems which requires RNAse III to cleave the pre-crRNA into multiple crRNAs, type V systems are capable of using the effector nuclease itself to cleave pre-crRNAs. Like Type-II CRISPR-Cas systems, Type V CRISPR-Cas systems are again known as DNA nucleases. Unlike Type II CRISPR-Cas systems, some Type V enzymes (e.g., Cas12a) appear to have a robust single-stranded nonspecific deoxyribonuclease activity that is activated by the first crRNA directed cleavage of a double-stranded target sequence.

Type VI CRIPSR-Cas systems have RNA-guided RNA endonucleases. Instead of RuvC-like domains, the single polypeptide effector of Type VI systems (e.g. Cas13) comprises two HEPN ribonuclease domains. Differing from both Type II and V systems, Type VI systems also appear to not need a tracrRNA for processing of pre-crRNA into crRNA. Similar to type V systems, however, some Type VI systems (e.g., C2C2) appear to possess robust single-stranded nonspecific nuclease (ribonuclease) activity activated by the first crRNA directed cleavage of a target RNA.

Because of their simpler architecture, Class II CRISPR-Cas have been most widely adopted for engineering and development as designer nuclease/genome editing applications.

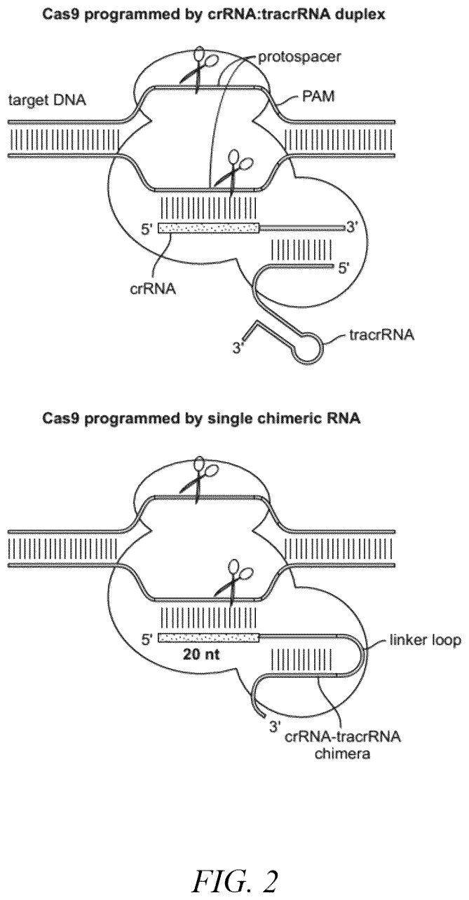

One of the early adaptations of such a system for in vitro use can be found in Jinek et al. (Science. 2012 Aug. 17; 337(6096):816-21, which is entirely incorporated herein by reference). The Jinek study first described a system that involved (i) recombinantly-expressed, purified full-length Cas9 (e.g., a Class II, Type II Cas enzyme) isolated from S. pyogenes SF370, (ii) purified mature ˜42 nt crRNA bearing a ˜20 nt 5′ sequence complementary to the target DNA sequence desired to be cleaved followed by a 3′ tracr-binding sequence (the whole crRNA being in vitro transcribed from a synthetic DNA template carrying a T7 promoter sequence); (iii) purified tracrRNA in vitro transcribed from a synthetic DNA template carrying a T7 promoter sequence, and (iv) Mg²⁺.Jinek later described an improved, engineered system wherein the crRNA of (ii) is joined to the 5′ end of (iii) by a linker (e.g., GAAA) to form a single fused synthetic guide RNA (sgRNA) capable of directing Cas9 to a target by itself (compare top and bottom panel of FIG. 2).

Mali et al. (Science. 2013 Feb. 15; 339(6121): 823-826.), which is entirely incorporated herein by reference, later adapted this system for use in mammalian cells by providing DNA vectors encoding (i) an ORF encoding codon-optimized Cas9 (e.g., a Class II, Type II Cas enzyme) under a suitable mammalian promoter with a C-terminal nuclear localization sequence (e.g., SV40 NLS) and a suitable polyadenylation signal (e.g., TK pA signal); and (ii) an ORF encoding an sgRNA (having a 5′ sequence beginning with G followed by 20 nt of a complementary targeting nucleic acid sequence joined to a 3′ tracr-binding sequence, a linker, and the tracrRNA sequence) under a suitable Polymerase III promoter (e.g., the U6 promoter).

Transposons are mobile elements that can move between positions in a genome. Such transposons have evolved to limit the negative effects they exert on the host. A variety of regulatory mechanisms are used to maintain transposition at a low frequency and sometimes coordinate transposition with various cell processes. Some prokaryotic transposons also can mobilize functions that benefit the host or otherwise help maintain the element. Certain transposons may have also evolved mechanisms of tight control over target site selection, the most notable example being the Tn7 family.

Transposon Tn7 and similar elements may be reservoirs for antibiotic resistance and pathogenesis functions in clinical settings, as well as encoding other adaptive functions in natural environments. The Tn7 system, for example, has evolved mechanisms to almost completely avoid integrating into important host genes, but also maximize dispersal of the element by recognizing mobile plasmids and bacteriophage capable of moving Tn7 between host bacteria.

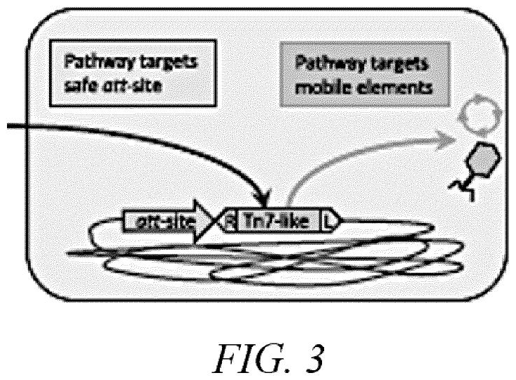

Tn7 and Tn7-like elements may control where and when they insert, possessing one pathway that directs insertion into a single conserved position in bacterial genomes and a second pathway that appears to be adapted to maximizing targeting into mobile plasmids capable of transporting the element between bacteria (see FIG. 3). The association between Tn7-like transposons and CRISPR-Cas systems suggests that the transposons might have hijacked CRISPR effectors to generate R-loops in target sites and facilitate the spread of transposons via plasmids and phages.

### MG36 Systems

In one aspect, the present disclosure provides for a system for transposing a cargo nucleotide sequence to a target nucleic acid site. This system may comprise a first double-stranded nucleic acid. The first double-stranded nucleic acid may comprise the cargo nucleotide sequence wherein the cargo nucleotide sequence is configured to interact with a recombinase complex. The system may comprise a Cas effector complex. This Cas effector complex may comprise a class II, type II Cas effector and at least one engineered guide polynucleotide configured to hybridize to the target nucleic acid site. The class II, type II Cas effector can comprise a RuvC domain and an HNH domain. The system may comprise the recombinase or transposase complex, wherein the recombinase or transposase complex is configured to recruit the cargo nucleotide sequence to the target nucleic acid site.

In some cases, the cargo nucleotide sequence is flanked by a left-hand transposase recognition sequence. In some cases, the cargo nucleotide sequence is flanked by a right-hand transposase recognition sequence. In some cases, the cargo nucleotide sequence is flanked by a left-hand transposase recognition sequence and a right-hand transposase recognition sequence. In some cases, the system further comprises a second double-stranded nucleic acid comprising the target nucleic acid site. In some cases, the system further comprises a PAM sequence compatible with the Cas effector complex adjacent to the target nucleic acid site. In some cases, the PAM sequence is located 3′ of the target nucleic acid site. In some cases, the recombinase or transposase complex is a Tn7 type transposase complex. In some cases, the engineered guide polynucleotide is configured to bind the class II, type II Cas effector. In some cases, the class II, type II Cas effector comprises a polypeptide which has at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 1 or a variant thereof. In some cases, the class II, type II Case effector comprises a polypeptide substantially identical to SEQ ID NO: 1.

In some cases, the recombinase or transposase complex comprises at least one polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 2-5 or a variant thereof. In some cases, the recombinase or transposase complex comprises at least one polypeptide comprising a sequence substantially identical to any one of SEQ ID NOs: 2-5. In some cases, the recombinase or transposase complex comprises at least two polypeptides comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 2-5 or a variant thereof. In some cases, the recombinase or transposase complex comprises at least two polypeptides comprising a sequence substantially identical to any one of SEQ ID NOS: 2-5 or a variant thereof. In some cases, the recombinase or transposase complex comprises at least three polypeptides comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID Nos: 2-5 or a variant thereof. In some cases, the recombinase or transposase complex comprises at least three polypeptides comprising a sequence substantially identical to any one of SEQ ID NOs: 2-5 or a variant thereof. In some cases, the recombinase or transposase complex comprises four polypeptides comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 2-5 or a variant thereof. In some cases, the recombinase or transposase complex comprises four polypeptides comprising a sequence substantially identical to any one of SEQ ID NOs: 2-5 or a variant thereof. In some cases, the recombinase or transposase complex comprises a TnsB1 polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 2 or a variant thereof. In some cases, the recombinase or transposase complex comprises a TnsB1 polypeptide comprising a sequence substantially identical to SEQ ID NOs: 2 or a variant thereof. In some cases, the recombinase or transposase complex comprises a TnsB2 polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NO: 3 or a variant thereof. In some cases, the recombinase or transposase complex comprises a TnsB2 polypeptide comprising a sequence substantially identical to SEQ ID NO: 3 or a variant thereof. In some cases, the recombinase or transposase complex comprises a TnsT1 polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NO: 4 or a variant thereof. In some cases, the recombinase or transposase complex comprises a TnsT1 polypeptide comprising a sequence substantially identical to SEQ ID NO: 4 or a variant thereof. In some cases, the recombinase or transposase complex comprises a TnsC polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NO: 5 or a variant thereof. In some cases, the recombinase or transposase complex comprises a TnsC polypeptide comprising a sequence substantially identical to SEQ ID NO: 5 or a variant thereof.

In some cases, the engineered guide polynucleotide comprises a sequence comprising at least 60-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 11 or a variant thereof.

In some cases, the left-hand recombinase sequence comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 17-18 or a variant thereof. In some cases, the right-had recombinase sequence comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 19 or a variant thereof.

In some cases, the class II, type II Cas effector and the recombinase or transposase complex are encoded by polynucleotide sequences comprising fewer than about 20 kilobases, fewer than about 15 kilobases, fewer than about 10 kilobases, or fewer than about 5 kilobases.

In one aspect, the present disclosure provides a method for transposing a cargo nucleotide sequence to a target nucleic acid site comprising a target nucleotide sequence comprising expressing a system described herein within a cell or introducing a system described herein to a cell.

### MG39 Systems

In one aspect, the present disclosure provides for a system for transposing a cargo nucleotide sequence to a target nucleic acid site. The system may comprise a first double-stranded nucleic acid comprising a cargo nucleotide sequence. This cargo nucleotide sequence may be configured to interact with a Tn7 type transposase complex. The system may comprise a Cas effector complex. The Cas effector complex may comprise a class II, type V Cas effector and an engineered guide polynucleotide configured to hybridize to the target nucleotide sequence. The class II, type V Cas effector can comprise a RuvC domain. The system may comprise a Tn7 type transposase complex configured to bind the Cas effector complex, wherein the Tn7 type transposase complex comprises a TnsA subunit.

In some cases, the cargo nucleotide sequence is flanked by a left-hand transposase recognition sequence. In some cases, the cargo nucleotide sequence is flanked by a right-hand transposase recognition sequence. In some cases, the cargo nucleotide sequence is flanked by a left-hand transposase recognition sequence and a right-hand transposase recognition sequence. In some cases, the system further comprises a second double-stranded nucleic acid comprising the target nucleic acid site. In some cases, the system further comprises a PAM sequence compatible with the Cas effector complex adjacent to the target nucleic acid site. In some cases, the PAM sequence is located 3′ of the target nucleic acid site.

In some cases, the engineered guide polynucleotide is configured to bind the class II, type V Cas effector. In some cases, the class II, type V Cas effector comprises a polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 5, or a variant thereof. In some cases, the class II, type V Cas effector comprises a polypeptide comprising a sequence substantially identical to SEQ ID NO: 5 or a variant thereof. In some cases, the TnsA subunit comprises a polypeptide having a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 7 or a variant thereof. In some cases, the TnsA subunit comprises a polypeptide having a sequence substantially identical to SEQ ID NO: 7 or a variant thereof.

In some cases, the Tn7 type transposase complex comprises at least one polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 8-10, or a variant thereof. In some cases, the recombinase or transposase complex comprises at least one polypeptide comprising a sequence substantially identical to any one of SEQ ID NOs: 8-10, or a variant thereof. In some cases, the Tn7 type transposase complex comprises at least two polypeptides comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 8-10, or a variant thereof. In some cases, the Tn7 type transposase complex comprises at least two polypeptides comprising a sequence substantially identical to any one of SEQ ID NOs: 8-10, or a variant thereof. In some cases, the Tn7 type transposase complex comprises at least three polypeptides comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 8-10 or a variant thereof. In some cases, the Tn7 type transposase complex comprises at least three polypeptides comprising a sequence substantially identical to any one of SEQ ID NOs: 8-10 or a variant thereof.

In some cases, the Tn7 type transposase complex comprises a TnsA polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 7 or a variant thereof. In some cases, the Tn7 type transposase complex comprises a TnsA polypeptide comprising a sequence substantially identical to any one of SEQ ID NOs: 7 or a variant thereof. In some cases, the Tn7 type transposase complex comprises a TnsB polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 8 or a variant thereof. In some cases, the Tn7 type transposase complex comprises a TnsB polypeptide comprising a sequence substantially identical to any one of SEQ ID NOs: 8 or a variant thereof. In some cases, the Tn7 type transposase complex comprises a TnsC polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 9 or a variant thereof. In some cases, the Tn7 type transposase complex comprises a TnsC polypeptide comprising a sequence substantially identical to any one of SEQ ID NOs: 9 or a variant thereof. In some cases, the Tn7 type transposase complex comprises a TniQ polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 10 or a variant thereof. In some cases, the Tn7 type transposase complex comprises a TniQ polypeptide comprising a sequence substantially identical to any one of SEQ ID NOs: 10 or a variant thereof.

In some cases, the engineered guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 13-16, or a variant thereof. In some cases, the engineered guide polynucleotide comprises a sequence comprising at least about 46-80 consecutive nucleotides substantially identical to any one of SEQ ID NOs: 13-16 or a variant thereof.

In some cases, the left-hand recombinase sequence comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 20, or a variant thereof. In some cases, the left-hand recombinase sequence comprises a sequence substantially identical to SEQ ID NO: 20, or a variant thereof.

In some cases, the right-hand recombinase sequence comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 21, or a variant thereof. In some cases, the right-hand recombinase sequence comprises a sequence substantially identical to SEQ ID NO: 21, or a variant thereof.

In some cases, the class II, type V Cas effector and the Tn7 type transposase complex are encoded by polynucleotide sequences comprising fewer than about 20 kilobases, fewer than about 15 kilobases, fewer than about 10 kilobases, or fewer than about 5 kilobases.

In one aspect, the present disclosure provides for a method for transposing a cargo nucleotide sequence to a target nucleic acid site comprising a target nucleotide sequence comprising expressing a system described herein within a cell or introducing a system described herein to a cell.

In one aspect, the present disclosure provides for a method for transposing a cargo nucleotide sequence to a target nucleic acid site, comprising contacting a first double-stranded nucleic acid comprising a cargo nucleotide sequence with a Cas effector complex. The Cas effector complex may comprise a class II, type II Cas effector and at least one engineered guide polynucleotide configured to hybridize to the target nucleic acid site. The method may comprise contacting the first double-stranded nucleic acid comprising the cargo nucleotide sequence with a recombinase or transposase complex configured to recruit the cargo nucleotide to the target nucleic acid site. The method may comprise contacting the first double-stranded nucleic acid comprising the cargo nucleotide sequence with a second double-stranded nucleic acid comprising the target nucleic acid site.

In some cases, the cargo nucleotide sequence is flanked by a left-hand transposase recognition sequence. In some cases, the cargo nucleotide sequence is flanked by a right-hand transposase recognition sequence. In some cases, the cargo nucleotide sequence is flanked by a left-hand transposase recognition sequence and a right-hand transposase recognition sequence. In some cases, the Cas effector complex further comprises a PAM sequence compatible with the Cas effector complex adjacent to the target nucleic acid site. In some cases, the PAM sequence is located 3′ of the target nucleic acid site. In some cases, the PAM sequence is located 5′ of the target nucleic acid site.

### MG64 Systems

In one aspect, the present disclosure provides for a system for transposing a cargo nucleotide sequence to a target nucleic acid site. The system may comprise a first double-stranded nucleic acid comprising a cargo nucleotide sequence. This cargo nucleotide sequence may be configured to interact with a Tn7 type transposase complex. The system may comprise a Cas effector complex. The Cas effector complex may comprise a class II, type V Cas effector and an engineered guide polynucleotide configured to hybridize to the target nucleotide sequence. The system may comprise a Tn7 type transposase complex configured to bind the Cas effector complex. The class II, type V Cas effector can comprise a RuvC domain.

In some cases, the cargo nucleotide sequence is flanked by a left-hand transposase recognition sequence. In some cases, the cargo nucleotide sequence is flanked by a right-hand transposase recognition sequence. In some cases, the cargo nucleotide sequence is flanked by a left-hand transposase recognition sequence and a right-hand transposase recognition sequence. In some cases, the system further comprises a second double-stranded nucleic acid comprising the target nucleic acid site. In some cases, the system further comprises a PAM sequence compatible with the Cas effector complex adjacent to the target nucleic acid site. In some cases, the PAM sequence is located 3′ of the target nucleic acid site. In some cases, the PAM sequence is located 5′ of the target nucleic acid site. In some cases, the PAM sequence comprises 5′-nGTn-3′ or 5′-nGTt-3′.

In some cases, the engineered guide polynucleotide is configured to bind the class II, type V Cas effector. In some cases, the class II, type V Cas effector comprises a polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs:22, 26, 30, 34, 55-89, 104, or 147, or a variant thereof. In some cases, the class II, type V Cas effector comprises a polypeptide comprising a sequence substantially identical to any one of SEQ ID NOs:22, 26, 30, 34, 55-89, 104, or 147, or a variant thereof.

In some cases, the Tn7 type transposase complex comprises at least one polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs:23-25, 27-29, 31-33, 35-37, 101-103, 105-107, or 148-150, or a variant thereof. In some cases, the recombinase or transposase complex comprises at least one polypeptide comprising a sequence substantially identical to any one of SEQ ID NOs:23-25, 27-29, 31-33, 35-37, 101-103, 105-107, or 148-150, or a variant thereof. In some cases, the Tn7 type transposase complex comprises at least two polypeptides comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 23-25, 27-29, 31-33, 35-37, 101-103, 105-107, or 148-150, or a variant thereof. In some cases, the Tn7 type transposase complex comprises at least two polypeptides comprising a sequence substantially identical to any one of SEQ ID NOs: 23-25, 27-29, 31-33, 35-37, 101-103, 105-107, or 148-150, or a variant thereof. In some cases, the Tn7 type transposase complex comprises at least three polypeptides comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs:23-25, 27-29, 31-33, 35-37, 101-103, 105-107, or 148-150, or a variant thereof. In some cases, the Tn7 type transposase complex comprises at least three polypeptides comprising a sequence substantially identical to any one of SEQ ID NOs: 23-25, 27-29, 31-33, 35-37, 101-103, 105-107, or 148-150, or a variant thereof.

In some cases, the Tn7 type transposase complex comprises TnsB, TnsC, and TniQ polypeptides comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs:23-25, 27-29, 31-33, 35-37, 101-103, 105-107, or 148-150, or a variant thereof, respectively. In some cases, the Tn7 type transposase complex comprises a TnsB polypeptide comprising a sequence substantially identical to any one of SEQ ID NOs: 8 or a variant thereof. In some cases, the Tn7 type transposase complex comprises TnsB, TnsC, and TniQ polypeptides comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs:23-25, 27-29, 31-33, 35-37, 101-103, 105-107, or 148-150, or a variant thereof, respectively.

In some cases, the engineered guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 90, 91, 92, 93, 117, 151, 156-181, or 209-234, or a variant thereof. In some cases, the engineered guide polynucleotide comprises a sequence comprising at least about 46-80 consecutive nucleotides substantially identical any one of SEQ ID NOs: 90, 91, 92, 93, 117, 151, 156-181, or 209-234, or a variant thereof.

In some cases, the engineered guide polynucleotide comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to non-degenerate nucleotides of any one of SEQ ID NOs: 111-114 or 201-204, or a variant thereof. In some cases, the engineered guide polynucleotide comprises a sequence comprising at least about 46-80 consecutive nucleotides substantially identical to the non-degenerate nucleotides of any one of SEQ ID NOs: 111-114 or 201-204, or a variant thereof.

In some cases, the left-hand recombinase sequence comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 125, 127, 123, 129, 131, 133, 153, or 134, or a variant thereof. In some cases, the left-hand recombinase sequence comprises a sequence substantially identical to any one of SEQ ID NOs: 125, 127, 123, 129, 131, 133, 153, or 134, or a variant thereof.

In some cases, the right-hand recombinase sequence comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 126, 155, 128, 124, 130, 132, or 154, or a variant thereof. In some cases, the right-hand recombinase sequence comprises a sequence substantially identical to any one of SEQ ID NOs: 126, 155, 128, 124, 130, 132, or 154, or a variant thereof.

In some cases, the class II, type V Cas effector and the Tn7 type transposase complex are encoded by polynucleotide sequences comprising fewer than about 20 kilobases, fewer than about 15 kilobases, fewer than about 10 kilobases, or fewer than about 5 kilobases.

### MG108 Systems

In one aspect, the present disclosure provides for a system for transposing a cargo nucleotide sequence to a target nucleic acid site. The system may comprise a first double-stranded configured to interact with a Tn7 type transposase complex. The system may comprise a Cas effector complex. The Cas effector complex may comprise a class II, type V Cas effector and an engineered guide polynucleotide configured to hybridize to the target nucleotide sequence. The class II, type V Cas effector can comprise a RuvC domain. The system may comprise a Tn7 type transposase complex configured to bind the Cas effector complex. In some cases, the Tn7 type transposase complex comprises TnsB and TnsC components but does not comprise a TnsA and/or TniQ component.

In some cases, the cargo nucleotide sequence is flanked by a left-hand transposase recognition sequence. In some cases, the cargo nucleotide sequence is flanked by a right-hand transposase recognition sequence. In some cases, the cargo nucleotide sequence is flanked by a left-hand transposase recognition sequence and a right-hand transposase recognition sequence. In some cases, the system further comprises a second double-stranded nucleic acid comprising the target nucleic acid site. In some cases, the system further comprises a PAM sequence compatible with the Cas effector complex adjacent to the target nucleic acid site. In some cases, the PAM sequence is located 3′ of the target nucleic acid site. In some cases, the PAM sequence is located 5′ of the target nucleic acid site.

In some cases, the engineered guide polynucleotide is configured to bind the class II, type V Cas effector. In some cases, the class II, type V Cas effector comprises a polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 38 or SEQ ID NO: 108, or a variant thereof. In some cases, the class II, type V Cas effector comprises a polypeptide comprising a sequence substantially identical to SEQ ID NO: 38 or SEQ ID NO: 108, or a variant thereof.

In some cases, the Tn7 type transposase complex comprises at least one polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 39-40 or 109-110, or a variant thereof. In some cases, the recombinase or transposase complex comprises at least one polypeptide comprising a sequence substantially identical to any one of SEQ ID NOs: 39-40 or 109-110, or a variant thereof. In some cases, the Tn7 type transposase complex comprises at least two polypeptides comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 39-40 or 109-110, or a variant thereof. In some cases, the Tn7 type transposase complex comprises at least two polypeptides comprising a sequence substantially identical to any one of SEQ ID NOs: 39-40 or 109-110, or a variant thereof. In some cases, the Tn7 type transposase complex comprises at least three polypeptides comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 39-40 or 109-110, or a variant thereof. In some cases, the Tn7 type transposase complex comprises at least three polypeptides comprising a sequence substantially identical to any one of SEQ ID NOs: 39-40 or 109-110, or a variant thereof.

In some cases, the Tn7 type transposase complex comprises a TnsB component comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 40 or 109, or a variant thereof. In some cases, the recombinase or transposase complex comprises a TnsB component comprising a sequence substantially identical to any one of SEQ ID NOs: 40 or 109, or a variant thereof.

In some cases, the Tn7 type transposase complex comprises a TnsC component comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NOs: 39 or 110, or a variant thereof. In some cases, the recombinase or transposase complex comprises a TnsC component comprising a sequence substantially identical to any one of SEQ ID NOs: 39 or 110, or a variant thereof.

In some cases, the Tn7 type transposase complex comprises TnsB and TnsC components comprising sequences having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NOs: 40 and 39 or 109 and 110, or a variant thereof, respectively. In some cases, the recombinase or transposase complex comprises TnsB and TnsC components comprising sequences substantially identical to any one of SEQ ID NOs: 40 and 39 or 109 and 110, or a variant thereof, respectively.

In some cases, the engineered guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 118, 182, 183, 235, or 236, or a variant thereof. In some cases, the engineered guide polynucleotide comprises a sequence comprising at least about 46-80 consecutive nucleotides substantially identical any one of SEQ ID NOs: 118, 182, 183, 235, or 236, or a variant thereof.

In some cases, the engineered guide polynucleotide comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to non-degenerate nucleotides of any one of SEQ ID NOs: 115, 116, 205, or 206, or a variant thereof. In some cases, the engineered guide polynucleotide comprises a sequence comprising at least about 46-80 consecutive nucleotides substantially identical to the non-degenerate nucleotides of any one of SEQ ID NOs: 115, 116, 205, or 206, or a variant thereof.

In some cases, the left-hand recombinase sequence comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NOs: 134, or a variant thereof. In some cases, the left-hand recombinase sequence comprises a sequence substantially identical to SEQ ID NO:134, or a variant thereof.

In some cases, the right-hand recombinase sequence comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 135, or a variant thereof. In some cases, the right-hand recombinase sequence comprises a sequence substantially identical to SEQ ID NO: 135, or a variant thereof.

In some cases, the class II, type V Cas effector and the Tn7 type transposase complex are encoded by polynucleotide sequences comprising fewer than about 20 kilobases, fewer than about 15 kilobases, fewer than about 10 kilobases, or fewer than about 5 kilobases.

### MG110 Systems

In one aspect, the present disclosure provides for a system for transposing a cargo nucleotide sequence to a target nucleic acid site. The system may comprise a first double-stranded configured to interact with a Tn7 type transposase complex. The system may comprise a Cas effector complex. The Cas effector complex may comprise a class I, type I Cas effector and an engineered guide polynucleotide configured to hybridize to the target nucleotide sequence. The system may comprise a Tn7 type transposase complex configured to bind the Cas effector complex.

In some cases, the cargo nucleotide sequence is flanked by a left-hand transposase recognition sequence. In some cases, the cargo nucleotide sequence is flanked by a right-hand transposase recognition sequence. In some cases, the cargo nucleotide sequence is flanked by a left-hand transposase recognition sequence and a right-hand transposase recognition sequence. In some cases, the system further comprises a second double-stranded nucleic acid comprising the target nucleic acid site. In some cases, the system further comprises a PAM sequence compatible with the Cas effector complex adjacent to the target nucleic acid site. In some cases, the PAM sequence is located 3′ of the target nucleic acid site.

In some cases, the engineered guide polynucleotide is configured to bind the class I, type I Cas effector. In some cases, the class I, type I Cas effector comprises a polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NO: 41-43, or 48-50, or a variant thereof. In some cases, the class I, type I Cas effector comprises a polypeptide comprising a sequence substantially identical to any one of SEQ ID NO: 41-43, or 48-50, or a variant thereof.

In some cases, the engineered guide polynucleotide is configured to bind the class I, type I Cas effector. In some cases, the class I, type I Cas effector comprises Cas6, Cas7, and Cas8 effectors comprising sequences having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NO: 41-43, or 48-50, or a variant thereof. In some cases, the class I, type I Cas effector comprises Cas6, Cas7, and Cas8 effectors comprising sequences substantially identical to any one of SEQ ID NO: 41-43, or 48-50, or a variant thereof.

In some cases, the Tn7 type transposase complex comprises at least one polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 44-47, or 51-54, or a variant thereof. In some cases, the recombinase or transposase complex comprises at least one polypeptide comprising a sequence substantially identical to any one of SEQ ID NOs: 44-47, or 51-54, or a variant thereof. In some cases, the Tn7 type transposase complex comprises at least two polypeptides comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 44-47, or 51-54, or a variant thereof. In some cases, the Tn7 type transposase complex comprises at least two polypeptides comprising a sequence substantially identical to any one of SEQ ID NOs: 44-47, or 51-54, or a variant thereof. In some cases, the Tn7 type transposase complex comprises at least three polypeptides comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 44-47, or 51-54, or a variant thereof. In some cases, the Tn7 type transposase complex comprises at least three polypeptides comprising a sequence substantially identical to any one of SEQ ID NOs: 44-47, or 51-54, or a variant thereof. In some cases, the Tn7 type transposase complex comprises four polypeptides comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 44-47, or 51-54, or a variant thereof, or a variant thereof. In some cases, Tn7 type transposase complex comprises four polypeptides comprising a sequence substantially identical to any one of SEQ ID NOs: 44-47, or 51-54, or a variant thereof.

In some cases, the Tn7 type transposase complex comprises TnsA, TnsB, TnsC, and TniQ components comprising sequences having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 44-47, or 51-54, or a variant thereof, respectively.

In some cases, the engineered guide polynucleotide comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to non-degenerate nucleotides of any one of SEQ ID NOs: 121, 122, 207, or 208, or a variant thereof. In some cases, the engineered guide polynucleotide comprises a sequence comprising at least about 46-80 consecutive nucleotides substantially identical to the non-degenerate nucleotides of any one of SEQ ID NOs: 121, 122, 207, or 208, or a variant thereof.

In some cases, the left-hand recombinase sequence comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 136 or 138, or a variant thereof. In some cases, the left-hand recombinase sequence comprises a sequence substantially identical to SEQ ID NO: 136 or 138, or a variant thereof.

In some cases, the right-hand recombinase sequence comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 137 or 139, or a variant thereof, or a variant thereof. In some cases, the right-hand recombinase sequence comprises a sequence substantially identical to SEQ ID NO: 137 or 139, or a variant thereof, or a variant thereof.

In some cases, the class I, type I Cas effector and the Tn7 type transposase complex are encoded by polynucleotide sequences comprising fewer than about 20 kilobases, fewer than about 15 kilobases, fewer than about 10 kilobases, or fewer than about 5 kilobases.

In one aspect, the present disclosure provides for a method for transposing a cargo nucleotide sequence to a target nucleic acid site comprising a target nucleotide sequence comprising expressing a system described herein within a cell or introducing a system described herein to a cell.

In accordance with IUPAC conventions, the following abbreviations are used throughout the examples:
A=adenine
C=cytosine
G=guanine
T=thymine
R=adenine or guanine
Y=cytosine or thymine
S=guanine or cytosine
W=adenine or thymine
K=guanine or thymine
M=adenine or cytosine
B=C, G, or T
D=A, G, or T
H=A, C, or T
V=A, C, or G

### EXAMPLES

### Example 1—(General Protocol) PAM Sequence Identification/Confirmation for Systems Described Herein

Putative endonucleases were expressed in an E. coli lysate-based expression system (myTXTL. Arbor Biosciences). PAM sequences were determined by sequencing plasmids containing randomly-generated potential PAM sequences that could be cleaved by the putative nucleases. In this system, an E. coli codon optimized nucleotide sequence encoding the putative nuclease was transcribed and translated in vitro from a PCR fragment under control of a T7 promoter. A second PCR fragment with a minimal CRISPR array composed of a T7 promoter followed by a repeat-spacer-repeat sequence was transcribed in the same reaction. Successful expression of the endonuclease and repeat-spacer-repeat sequence in the TXTL system followed by CRISPR array processing provided active in vitro CRISPR nuclease complexes.

A library of target plasmids containing a spacer sequence matching that in the minimal array preceded by 8N mixed bases (potential PAM sequences) was incubated with the output of the TXTL reaction. After 1-3 hr, the reaction was stopped and the DNA was recovered via a DNA clean-up kit, e.g . . . . Zymo DCC, AMPure XP beads, QiaQuick etc. Adapter sequences were blunt-end ligated to DNA with active PAM sequences that were cleaved by the endonuclease, whereas DNA that was not cleaved is inaccessible for ligation. DNA segments comprising active PAM sequences were then amplified by PCR with primers specific to the library and the adapter sequence. The PCR amplification products were resolved on a gel to identify amplicons that correspond to cleavage events. The amplified segments of the cleavage reaction were also used as templates for preparation of an NGS library or as a substrate for Sanger sequencing. Sequencing this resulting library, which is a subset of the starting 8N library, revealed sequences with PAM activity compatible with the CRISPR complex. For PAM testing with a processed RNA construct, the same procedure was repeated except that an in vitro transcribed RNA was added along with the plasmid library and the minimal CRISPR array template is omitted.

For endonucleases which are binding-competent but nuclease deficient, the PAM was determined via a modification of the above procedure. After expression in TXTL, the sgRNA or crRNA and PAM library were added. Upon binding of the effector in a sgRNA-dependent manner to the spacer sequence, the spacer sequence was sequestered within the effector protein. The appropriate restriction enzyme that targets within the spacer sequence was added and all unprotected plasmids within the library were cleaved. The uncleaved (endonuclease-bound) members of the library which contain the PAM were identified by PCR and subsequent NGS library preparation of the band.

### Example 2—In Vitro Targeted Integrase Activity

Integrase activity was preferentially assayed with a previously identified PAM but may be conducted with a PAM library substrate instead, with reduced efficiency. One arrangement of components for in vitro testing involved three plasmids other than that containing the donor sequence: (1) an expression plasmid with effector (or effectors) under a T7 promoter: (2) an expression plasmid with integrase genes under a T7 promoter: a sgRNA or crRNA and tracrRNA: (3) a target plasmid which contained the spacer site and appropriate PAM; and (4) a donor plasmid which contained the required left end (LE) and right end (RE) DNA sequences for transposition around a cargo gene (e.g. a selection marker such as a Tet resistance gene). Using an in vitro transcription/translation (TXTL) system (e.g. E. coli lysate- or reticulocyte lysate-based system), the effector and integrase genes were expressed. After expression, the RNA, target DNA, and donor DNA were added and incubated to allow for transposition to occur. Transposition was detected via PCR across the junction of the integrase site, with one primer on the target DNA and one primer on the donor DNA. The resulting PCR product was sequenced via NGS to determine the exact insertion topology relative to the sgRNA/crRNA targeted site. The primers were located downstream such that a variety of insertion sites can be accommodated and detected. Primers were designed such that integration is detected in either orientation of cargo or on either side of the spacer, as the integration direction was also not known initially.

Integration efficiency was measured via quantitative PCR (qPCR) measurements of the experimental output of target DNA with integrated cargo, normalized to the amount of unmodified target DNA also measured via qPCR.

This assay may be conducted with purified protein components rather than from lysate-based expression. In this case the proteins were expressed in an E. coli protease deficient B strain under a T7 inducible promoter, the cells were lysed using sonication, and the His-tagged protein of interest was purified using HisTrap FF (GE Lifescience) Ni-NTA affinity chromatography on the AKTA Avant FPLC (GE Lifescience). Purity was determined using densitometry in ImageLab software (Bio-Rad) of the protein bands resolved on SDS-PAGE and InstantBlue Ultrafast (Sigma-Aldrich) coomassie stained acrylamide gels (Bio-Rad). The protein was desalted in storage buffer composed of 50 mM Tris-HCl, 300 mM NaCl, 1 mM TCEP, 5% glycerol; pH 7.5 (or other buffers as determined for maximum stability) and stored at −80° C. After purification the effector(s) and integrase(s) were added to the sgRNA, target DNA, and donor DNA as described above in a reaction buffer, for example 26 mM HEPES pH 7.5, 4.2 mM TRIS pH 8, 50 μg/mL BSA, 2 mM ATP, 2.1 mM DTT, 0.05 mM EDTA, 0.2 mM MgCl₂, 28 mM NaCl, 21 mM KCl, 1.35% glycerol, (final pH 7.5) supplemented with 15 mM Mg(OAc)₂.

### Example 3—Predicted RNA Folding

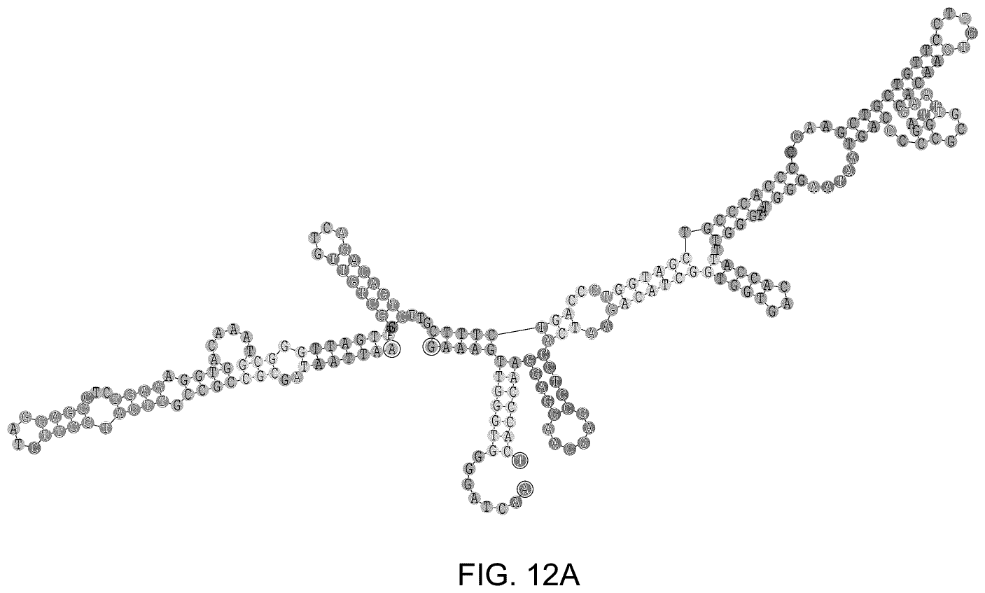

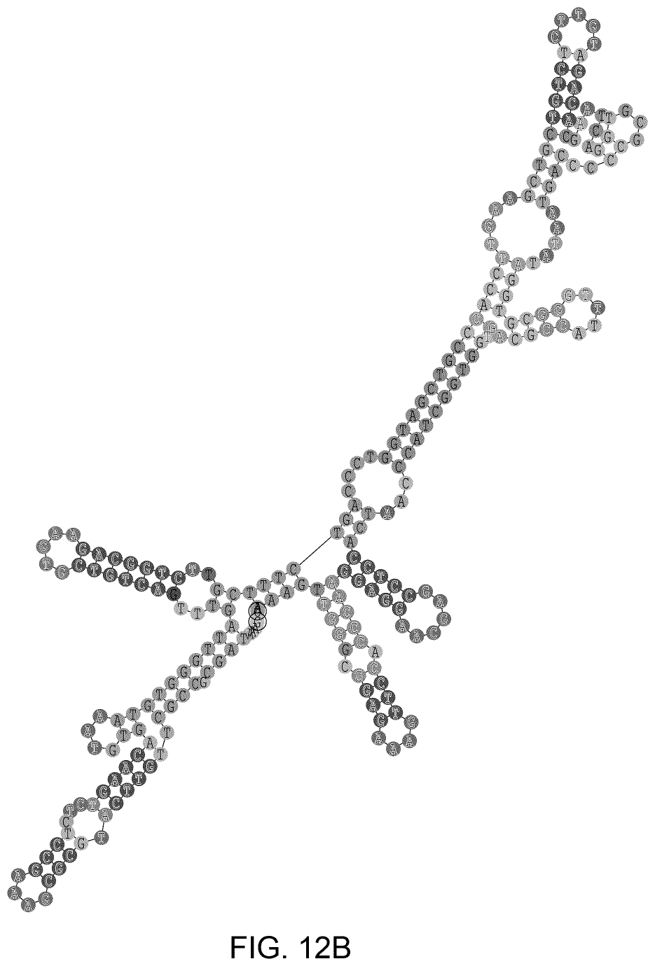

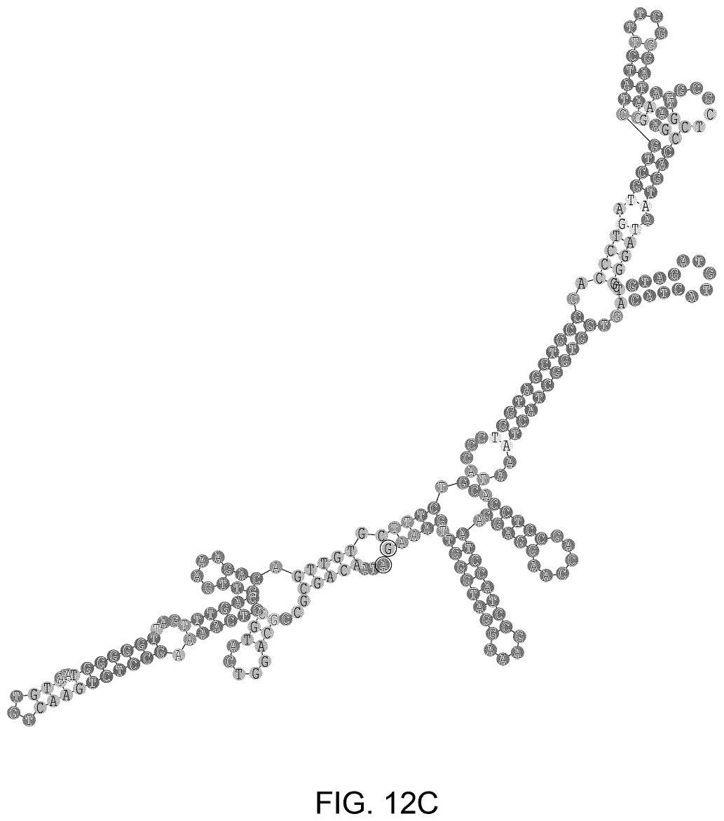

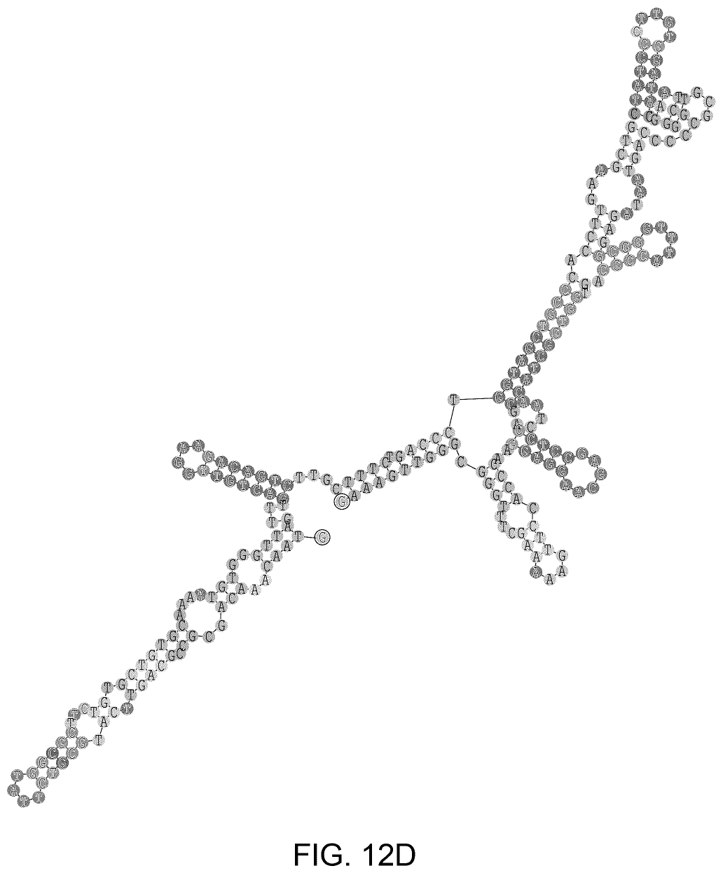

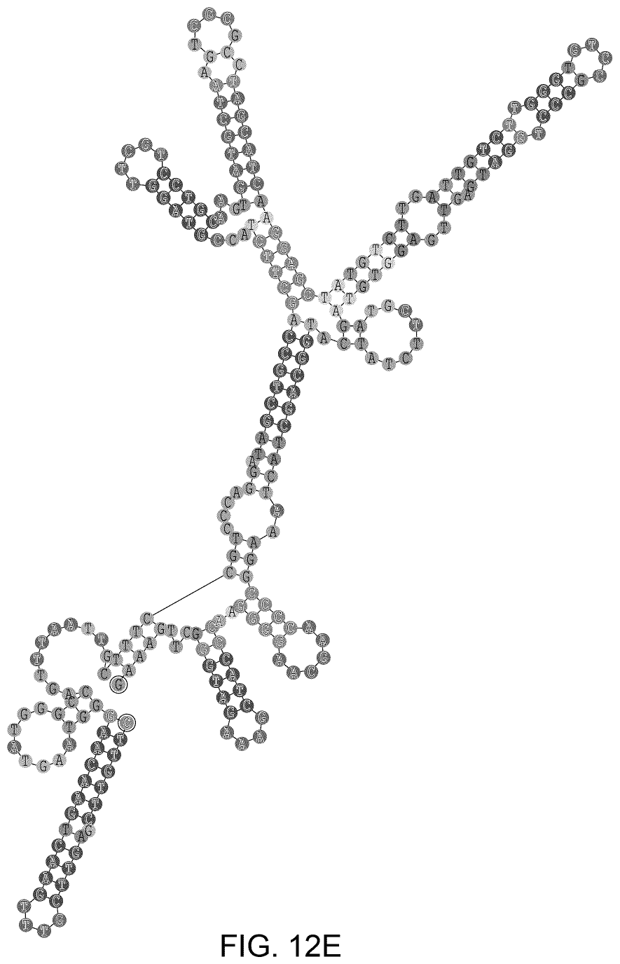

Predicted RNA folding of the active single RNA sequence was computed at 37 using the method of Andronescu 2007. All hairpin-loop secondary structures were singly deleted from the structure and iteratively compiled into a smaller single guide. In a second approach, the tracrRNA of MG64-1 was aligned to known type V-k tracrRNA, and areas of unique insertions were mutated out of the single guide and minimized by 57 bases. FIG. 12A depicts the predicted structure of MG64-2 sgRNA (SEQ ID NO:202). FIG. 12B depicts the predicted structure of MG64-4 sgRNA (SEQ ID NO:203). FIG. 12C depicts the predicted structure of MG64-6 sgRNA (SEQ ID NO:201). FIG. 12D depicts the predicted structure of MG64-7 sgRNA (SEQ ID NO:204). FIG. 12E depicts the predicted structure of MG108-1 sgRNA (SEQ ID NO:206). The shading of the bases corresponds to the probability of base pairing of that base.

### Example 4—Transposon End Verification Via Gel Shift

The transposon ends were tested for TnsB binding via an electrophoretic mobility shift assay (EMSA). In this case the potential LE or RE was synthesized as a DNA fragment (100-500 bp) and end-labeled with FAM via PCR with FAM-labeled primers. The TnsB protein was synthesized in an in vitro transcription/translation system (e.g. PURExpress). After synthesis, 1 μL of TnsB protein was added to 50 nM of the labeled RE or LE in a 10 μL reaction in binding buffer (20 mM HEPES pH 7.5, 2.5 mM Tris pH 7.5, 10 mM NaCl, 0.0625 mM EDTA, 5 mM TCEP, 0.005% BSA, 1 μg/mL poly(dI-dC), and 5% glycerol). The binding was incubated at 30 for 40 minutes, then 2 μL of 6× loading buffer (60 mM KCl, 10 mM Tris pH 7.6, 50% glycerol) was added. The binding reaction was separated on a 5% TBE gel and visualized. Shifts of the LE or RE in the presence of TnsB were attributed to successful binding and were indicative of transposase activity.

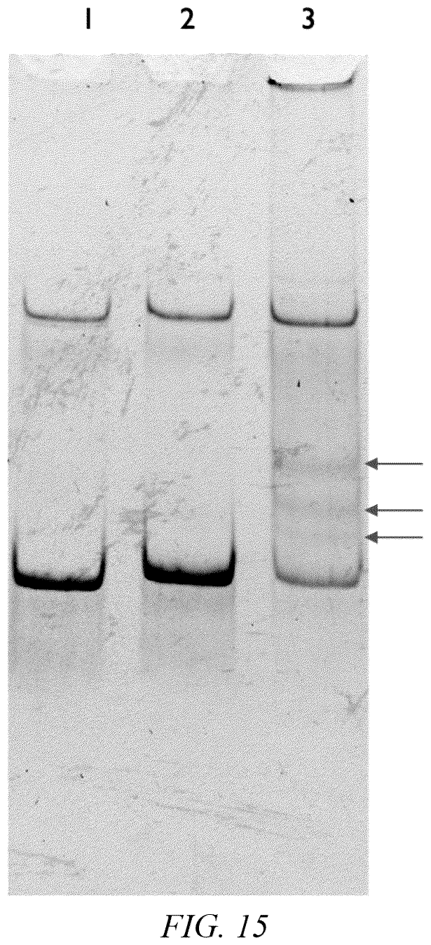

FIG. 15 shows an example of this experiment, where the RE DNA sequence for MG64-2 (e.g. SEQ ID NO:155) was end-labeled with FAM by the above procedure and incubated with the corresponding MG64-2 TnsB-like component (e.g. SEQ ID NO: 23). Upshift of the labeled band in Lane 3 indicates binding of the RE sequence by TnsB, indicating it contains an active RE transposition sequence.

### Example 5—Integrase Activity in E. coli (Prophetic)

As E. coli lacks the capacity to efficiently repair genomic double-stranded DNA breaks, transformation of E. coli by agents able to cause double-stranded breaks in the E. coli genome causes cell death. Exploiting this phenomenon, endonuclease or effector-assisted integrase activity is tested in E. coli by recombinantly expressing either the endonuclease or effector-assisted integrase and a guide RNA (determined e.g. as in Example 3) in a target strain with spacer/target and PAM sequences integrated into its genomic DNA.

Engineered strains are then transformed with a plasmid containing the nuclease or effector with single guide RNA, a plasmid expressing the integrase and accessory genes, and a plasmid containing a temperature sensitive origin of replication with a selectable marker flanked by left end (LE) and right end (RE) transposon motifs for integration. Transformants induced for expression of these genes are then screened for transfer of the marker to the genomic target by selection at restrictive temperature for plasmid replication and the marker integration in the genome is confirmed by PCR.

Off-target integrations are screened using an unbiased approach. In brief, purified gDNA is fragmented with Tn5 integrase or shearing, and DNA of interest is then PCR amplified using primers specific to a ligated adaptor and the selectable marker. The amplicons are then prepared for NGS sequencing. Analysis of the resulting sequences is trimmed of the transposon sequences and flanking sequences are mapped to the genome to determine insertion position, and off target insertion rates are determined.

### Example 6—Colony PCR Screen of Transposase Activity (Prophetic)

For testing of nuclease or effector assisted integrase activity in bacterial cells, strain MGB0032 is constructed from BL21(DE3) E. coli cells which are engineered to contain the target and corresponding PAM sequence specific to MG64_1. MGB0032 E. coli cells are then transformed with pJL56 (plasmid that expresses the MG64_1 effector and helper suite, ampicillin resistant) and pTCM 64_1 sg, a chloramphenicol-resistant plasmid that expresses the single guide RNA sequence for the engineered target of interest driven by a T7 promoter.

An MGB0032 culture containing both plasmids is then grown to a saturation, diluted at least 1:10 into growth culture with appropriate antibiotics, and incubated at 37° C. until OD of approximately 1. Cells from this growth stage are made electrocompetent and transformed with streamlined 64_1 pDonor, a plasmid bearing a tetracycline resistance marker flanked by left end (LE) and right end (RE) transposon motifs for integration. Electroporated cells are then recovered for 2 hours on LB medium in the presence or absence of IPTG at a final concentration of 100 UM before being plated on LB-agar-ampicillin-chloramphenicol-tetracycline and incubated 4 days at 37° C. Sterile toothpicks are used to sample each resultant CFU, which is mixed into water. To this solution is added Q5 High Fidelity PCR mastermix (New England Biolabs) and primers LA155 (5′-GCTCTTCCGATCTNNNNNGATGAGCGCATTGTTAGATTTCAT-3′) and oJL50 (5′-AAACCGACATCGCAGGCTTC-3′). These primers flank the predicted insertion junction. The predicted product size is 609 bp. DNA amplified PCR product is visualized on a 2% agarose gel. Sanger sequencing of PCR products confirms the transposition event.

### Example 7—In Cell Expression/In Vitro Assay (Prophetic)

To test the functionality of the NLS constructs in a physiologically relevant environment, constructs cloned with active NLS-tagged CAST components are integrated into K562 cells using lentiviral transduction. Briefly, constructs cloned into lentiviral transfer plasmids are transfected into 293T cells with envelope and packaging plasmids, and virus containing supernatant is harvested from the media after 72 hr incubation. Media containing virus is then incubated with K562 cell lines with 8 μg/mL of polybrene for 72 hrs, and transfected cells are then selected for integration in bulk using Puromycin at 1 μg/mL for 4 days. Cell lines undergoing selection are harvested at the end of 4 days, and differentially lysed for nuclear and cytoplasmic fractions. Subsequent fractions are then tested for transposition capability with a complementary set of in vitro expressed components.

10 million cells are centrifuged and washed once with 1×PBS pH7.4. Supernatant wash is aspirated completely to the cell pellet, and flash frozen at −80° C. for 16 hrs. After thawing on ice, cell pellet size is measured by mass, and appropriate extraction volumes of cell fractionation and nuclear extraction reagent (NE-PER) is used to natively extract proteins in cell fractions. Briefly, cytoplasmic extraction reagent is used at 1:10 mass of cells to volume of extraction reagent. Cell suspension is mixed by vortexing and lysed with non-ionic detergent. Cells are then centrifuged at 16.000×g at 4° C. for 5 minutes. Cytoplasmic extraction supernatant is then decanted and saved for in vitro testing. Nuclear extraction reagent is then added 1:2 original cell mass to nuclear extraction reagent and incubated on ice for 1 hr on ice with intermittent vortexing. Nuclear suspension is then centrifuged at 16,000×g for 10 minutes at 4° C. and supernatant nuclear extract is decanted and tested for in vitro transposition activity. Using 4 μL of each cell and nuclear extract for each condition, we perform the in vitro transposition reaction with a complementary set of in vitro expressed proteins, donor DNA, pTarget, and buffer. Evidence of transposition activity is assayed by PCR amplification of donor-target junctions.

### Example 8—Activity in Mammalian Cells (Prophetic)

To show targeting and cleavage activity in mammalian cells, nuclear localization sequences are fused to the C terminus of each of the nuclease or effector proteins and integrase proteins and the fusion proteins are purified. A single guide RNA targeting a genomic locus of interest is synthesized and incubated with the nuclease/effector protein to form a ribonucleoprotein complex. Cells are transfected with a plasmid containing a selectable neomycin resistance marker (NeoR) or a fluorescent marker flanked by the left end (LE) and right end (RE) motifs, recovered for 4-6 hours, and subsequently electroporated with nuclease RNP and integrase proteins. Integration of a plasmid into the genome is quantified by counting G418-resistant colonies or fluorescence activated cell cytometry. Genomic DNA is extracted 72 hours after electroporation and used for the preparation of an NGS-library. Off target frequency is assayed by fragmenting the genome and preparing amplicons of the transposon marker and flanking DNA for NGS library preparation. At least 40 different target sites are chosen for testing each targeting system's activity.

### Example 9—Activity of Targeted Nuclease

In situ expression and protein sequence analyses suggest that some RNA guided effectors are active nucleases. They contain predicted endonuclease-associated domains (matching RuvC and HNH_endonuclease domains) and predicted HNH and RuvC catalytic residues (see e.g. FIG. 4A, which shows predicted catalytic residues of the MG36-5 effector).

Candidate activity is tested with engineered single guide RNA sequences using the my TXTL system and in vitro transcribed RNA. Active proteins are identified as those that successfully cleave the library to yield a band around 170 bp in agarose gel electrophoresis

### Example 10—Identification of Transposons

Transposons are predicted to be active when they contain one or more protein sequences with integrase and/or integrase function between the left and right ends of the transposon. A typical Tn7 transposon generally comprises a catalytic integrase TnsB, but may also contain TnsA, TnsC, TnsD, TnsE, TniQ, and/or other integrases or integrases. The transposon ends comprise predicted integrase binding sites, which contain direct and/or inverted repeats of 15 bp to 150 bp in length flanking the integrase proteins and other ‘cargo’ genes. Protein sequence analysis indicated that the integrases contain integrase domains, integrase domains and/or integrase catalytic residues, suggesting that they are active (e.g. FIG. 4A, which shows a locus diagram for an example MG36-5 effector-based CAST system containing TnsB elements; and FIG. 5A, which shows a locus diagram for an exemplary MG39-1 effector-based CAST system containing TnsA, TnsB, TnsC, and TniQ elements).

### Example 11—Identification of CRISPR-Associated Transposons

Putative CRISPR-associated transposons (CAST) contain a DNA and/or RNA targeting CRISPR effector and proteins with predicted integrase function in the vicinity of a CRISPR array. In some systems, the effector is predicted to have nuclease activity based on the presence of endonuclease-associated catalytic domains and/or catalytic residues (e.g. FIG. 4A, which shows predicted catalytic residues of the MG36-5 effector in the context of a CAST system locus containing TnsB elements). The integrases were predicted to be associated with the active nucleases when the CRISPR loci (CRISPR nuclease and array) and the integrase proteins are located between the predicted transposon left and right ends (e.g. FIGS. 4B and 4C). In this case, the effector was predicted to direct DNA integration to specific genomic locations based on a guide RNA.

In some systems, the effector was predicted to have homology with known CRISPR effector proteins, but to be inactive based on the absence of endonuclease domains and/or catalytic residues (FIG. 5A). The integrases are predicted to be associated with the effector when the CRISPR loci (inactive CRISPR nuclease and array) and the integrase proteins are located within the predicted transposon left and right ends (FIGS. 5A and 5B).

### Example 12—CAST Discovery

CRISPR-associated transposons (CAST) are systems that comprise a transposon that has evolved to interact with a CRISPR system to promote targeted integration of DNA cargo.

CASTs are genomic sequences encoding one or more protein sequences involved in DNA transposition within the signature left and right ends of the transposon. A typical Tn7 transposon, generally comprises a catalytic transposase TnsB, but may also contain a catalytic transposase TnsA, a loader protein TnsC or TniB, and target recognition proteins TnsD, TnsE, TniQ, and/or other transposon-associated components. The transposon ends comprise predicted transposase binding sites, which contain direct and/or inverted repeats of 15 bp to 150 bp in length flanking the transposon machinery and other cargo genes.

In addition, CASTs also encode a DNA and/or RNA targeting CRISPR nuclease or effector in the vicinity of a CRISPR array. In some systems, the effector is predicted to be an active nuclease based on the presence of endonuclease-associated catalytic domains and/or catalytic residues. In some systems, the effector was predicted to have sequence similarity with known CRISPR effector proteins, but to be inactive based on the absence of endonuclease domains and/or catalytic residues. The transposons are predicted to be associated with the effector when the CRISPR locus and the transposon-associated proteins are located within the predicted transposon left and right ends. In this case, the effector is predicted to direct DNA integration to specific genomic locations based on a guide RNA.

### Example 13a—Cas12k CAST

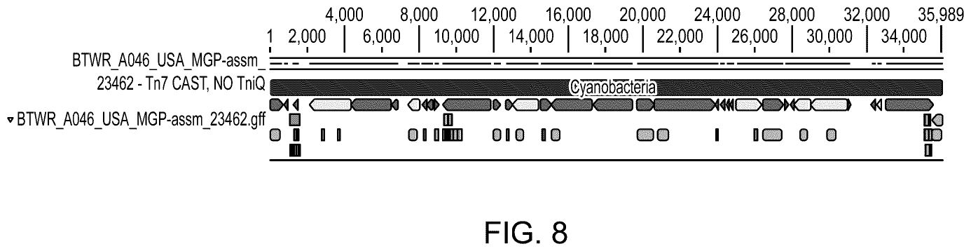

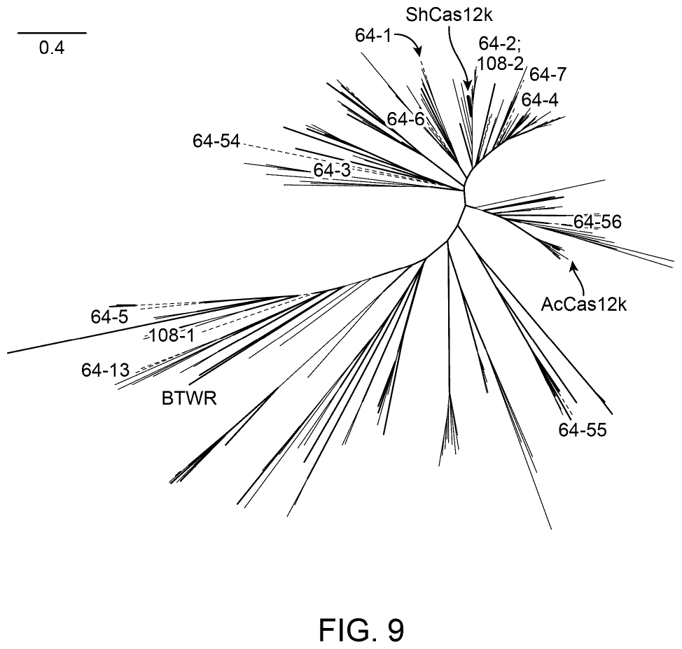

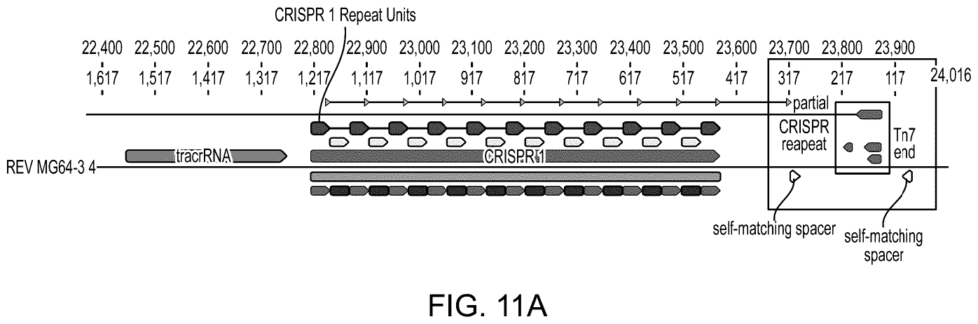

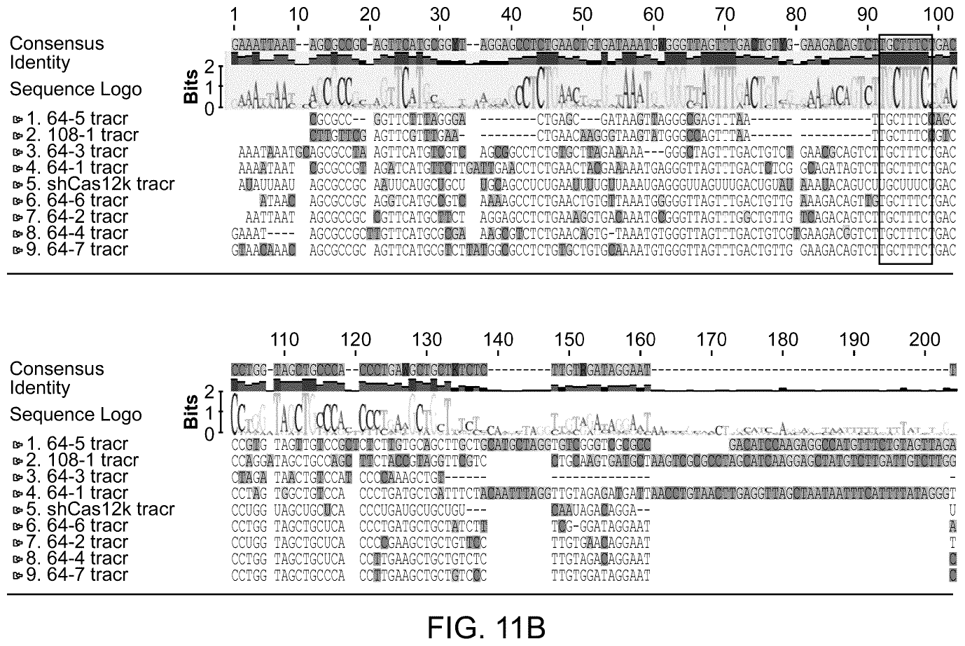

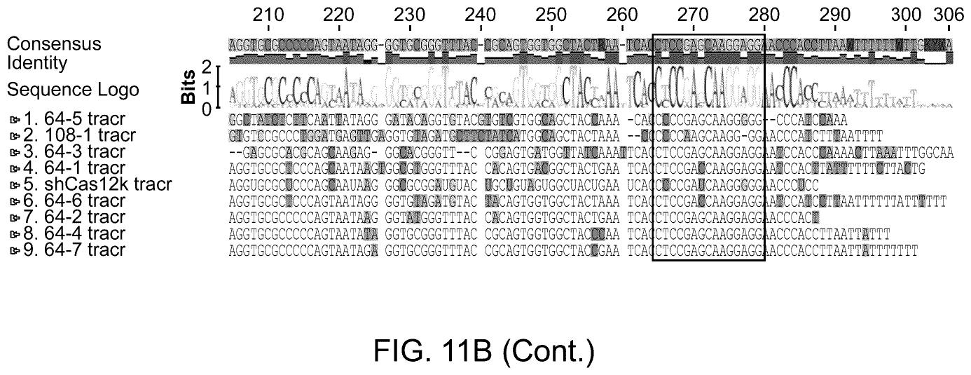

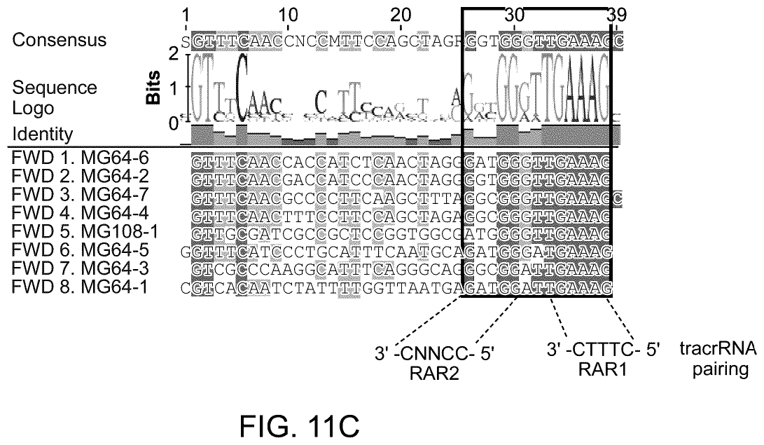

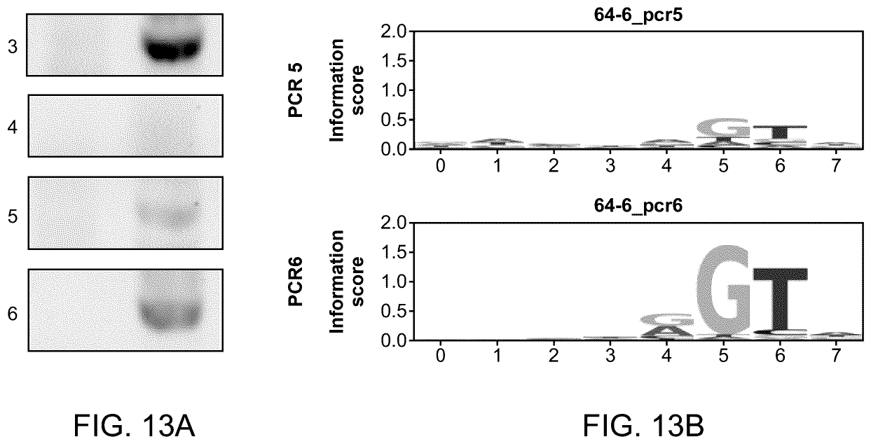

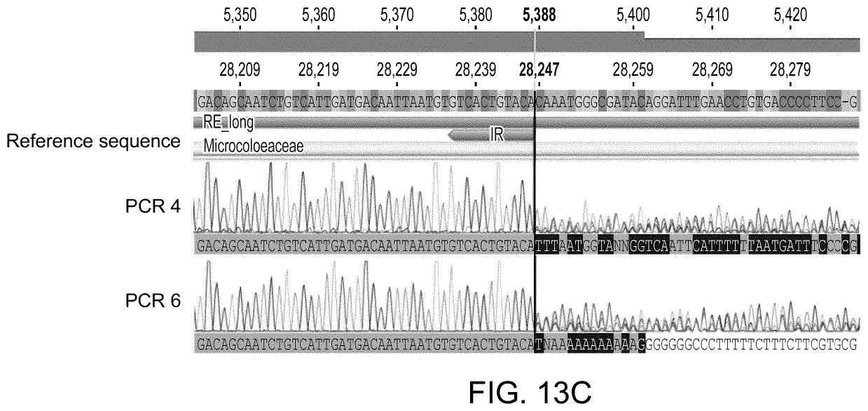

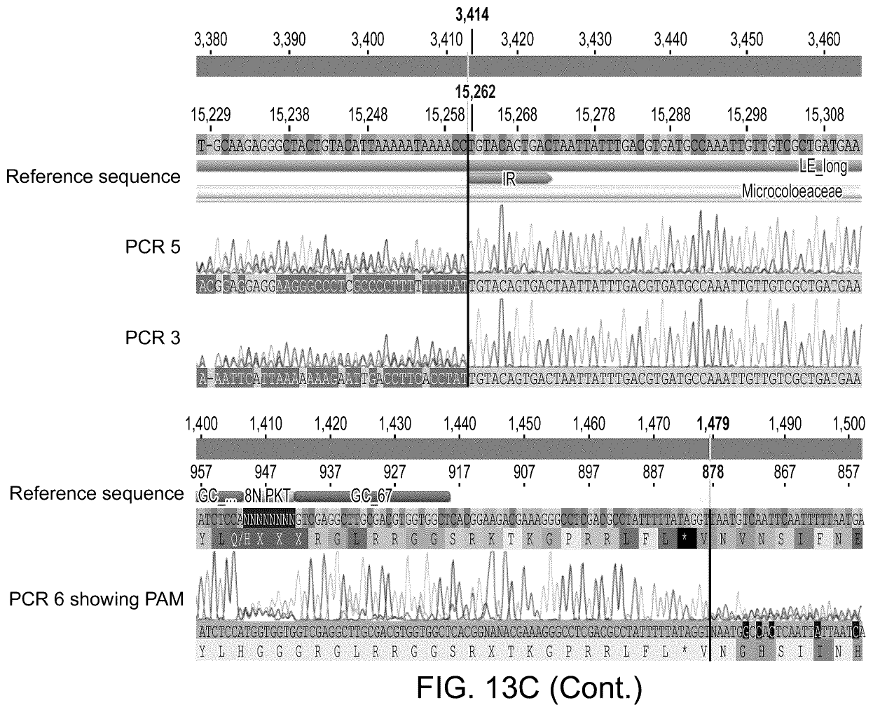

Cas 12k CAST systems encode a nuclease-defective CRISPR Cas12k effector, a CRISPR array, a tracrRNA, and Tn7-like transposition proteins (see e.g., FIG. 8, which shows a locus organization diagram for MG108-1 CAST system containing Cas12k). Cas12k effectors are phylogenetically diverse and features that confirm their association with CASTs have been confirmed for several (see e.g., FIG. 9, which shows how MG64-1, MG64-2, MG64-3, MG64-5. MG64-6, MG64-7, MG64-13, MG64-54, MG64-56, MG108-1, and MG108-2 effectors are part of this group). One such characteristic feature was transposon ends identified in the context of the MG64-3 CRISPR locus; the transposon left end was identified downstream from the MG64-3 CRISPR locus, as shown by terminal inverted repeats and self-matching spacer sequences (FIG. 11A). Another such characteristic that was identified includes Cas12k CAST CRISPR repeats (crRNA) which contain a conserved motif 5′-GNNGGNNTGAAAG-3′ (see e.g. MG64-2, MG64-4, MG64-5, MG64-6, MG64-7, and MG108-1 and FIG. 11B). Short repeat-antirepeats (RAR) within the crRNA motif aligned with different regions of the tracrRNA, and RAR motifs appeared to define the start and end of the tracrRNA. FIG. 13C shows presence of these RAR motifs in e.g. MG64-2, MG64-4, MG64-5, MG64-6, MG64-7, and MG108-1 families.

### Example 13b—Class I Type I-F CAST

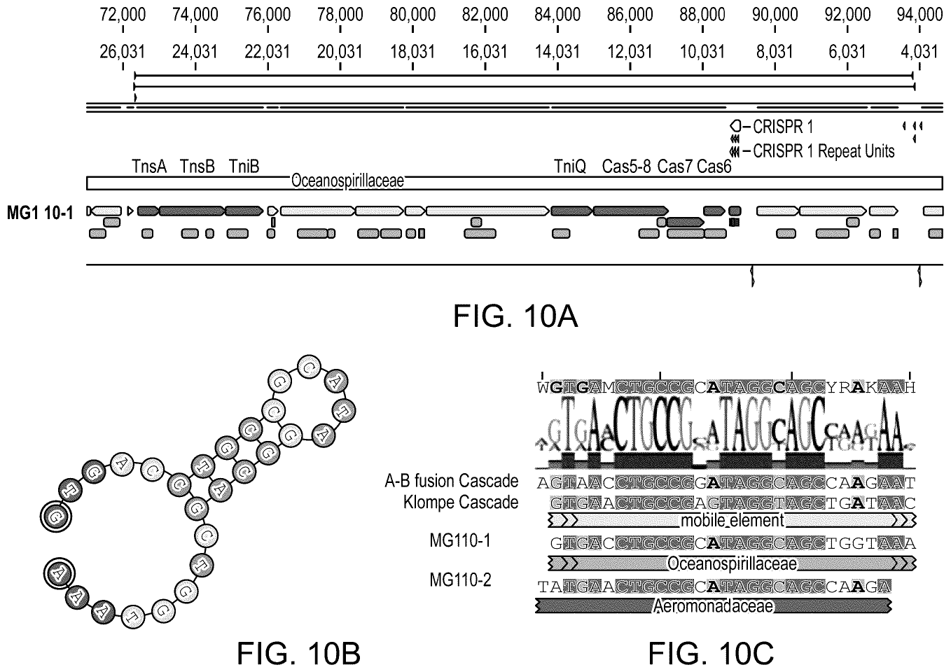

Some CASTs encode nuclease-defective CRISPR Type I-F Cascade effector proteins, a CRISPR array, and Tn7-like transposition proteins (see e.g. FIG. 10A, which shows a locus organization diagram of a MG110-1 effector-based Type I-F CAST system). Type I-F Cascade CAST were predicted to function with a single guide RNA encoded by the crRNA, which contains a conserved motif 5′-CTGCCGNNTAGGNAGC-3′ likely involved in formation of a stem-loop structure (see e.g., FIG. 10B-C, which shows an alignment of this feature in MG110-1 and MG110-2 family crRNAs SEQ ID NOs: 207 and 208). Based in part on its having these same features, the MG110-2 effector-containing and family was also identified as a Type I-F CAST system.

### Example 14—Transposon End Prediction

Transposon ends were estimated from intergenic regions flanking the effector and the transposon machinery. For example, for Cas12k CAST, the intergenic region located directly upstream from TnsB and directly downstream from the CRISPR locus, were predicted as containing the Tn7 transposon left and right ends (LE and RE) (see e.g. FIG. 11A, which shows LE and RE analysis in the context of an MG64-3 family CAST locus diagram).

Direct and inverted repeats (DR/IR) of ˜12 bp were predicted on the contig, with up to 2 mismatches. In addition, the Dotplot algorithm was used to find short (˜10-20 bp) DR/IR flanking CAST transposons. Matching DR/IR located in intergenic regions flanking CAST effector and transposon genes were predicted to encode transposon binding sites. LE and RE extracted from intergenic regions, which encode putative transposon binding sites, were aligned to define the transposon ends boundaries. Putative transposon LE and RE ends are identified as regions: a) located within 400 bp upstream and downstream from the first and last predicted transposon encoded genes: b) sharing multiple short inverted repeats; and c) sharing >65% nucleotide id. This process was repeated to identify putative LE/RE sequences for MG36-5 (SEQ ID NOs: 17-18). MG39-1 (SEQ ID NOs:20-21). MG64-2 (SEQ ID NOs: 125-126). MG64-4 (SEQ ID NOs: 127-128). MG64-6 (SEQ ID NOs: 123-124). MG64-7 (SEQ ID NOs: 129-130). MG64-13 (SEQ ID NOs: 131-132). MG64-54 (SEQ ID NO: 133). MG108-1 (SEQ ID NOs:134-135). MG110-1 (SEQ ID NOs: 136-137), and MG110-2 (SEQ ID NOs:138-139).

### Example 15—Single Guide Design for Class II, Type V CAST Systems

Analysis of the intergenic regions surrounding the Cas effector and CRISPR array for MG64 sub-families identified a potential anti-repeat sequence and a conserved “CYCC(N6)GGRG” stem-loop structure neighboring the antirepeat corresponding to the sequence of the tracrRNA (FIG. 11B). TracrRNA and crRNA repeat were folded and trimmed, adding a tetraloop sequence of GAAA to maintain the stem loop region of the crRNA-tracrRNA complementary sequence, in order to generate the sgRNA. These sequences are outlined in Table 1 below.

TABLE 1
Corresponding crRNA-tracrRNA sequences for MG
families described herein.

| | SEQ ID | |
|---|---|---|
| Description | NO: | Sequence |
| MG64-2 crRNA | 255 | See sequence listing |
| MG64-2 tracrRNA | 262 | AAUUAAUAGCGCCGCCGUUCAU |
| | | GCUUCUAGGAGCCUCUGAAAGG |
| | | UGACAAAUGCGGGUUAGUUUGG |
| | | CUGUUGUCAGACAGUCUUGCUU |
| | | UCUGACCCUGGUAGCUGCCCAC |
| | | CCCGAAGCUGCUGUUCCUUGUG |
| | | AACAGGAAUUAGGUGCGCCCCC |
| | | AGUAAUAAGGGUAUGGGUUUAC |
| | | CACAGUGGUGGCUACUGAAUCA |
| | | CCUCCGAGCAAGGAGGAACCCA |
| | | CU |
| MG64-4 crRNA | 256 | See sequence listing |
| MG64-4 tracrRNA | 209 | See sequence listing |
| MG64-6 crRNA | 257 | See sequence listing |
| MG64-6 tracrRNA | 263 | AUAACAGCGCCGCAGGUCAUGC |
| | | CGUCAAAAGCCUCUGAACUGUG |
| | | UUAAAUGGGGGUUAGUUUGACU |
| | | GUUGAAAGACAGUUGUGCUUUC |
| | | UGACCCUGGUAGCUGCCCACCC |
| | | UGAUGCUGCUAUCUUUCGGGAU |
| | | AGGAAUAAGGUGCGCUCCCAGU |
| | | AAUAGGGGUGUAGAUGUACUAC |
| | | AGUGGUGGCUACUAAAUCACCU |
| | | CCGACCAAGGAGGAAUCCAUCC |
| | | UUAAUUUUUUAUUUUUU |
| MG64-7 crRNA | 258 | See sequence listing |
| MG64-7 tracrRNA | 210 | See sequence listing |
| MG108-1 crRNA | 261 | See sequence listing |
| MG108-1 tracrRNA | 235 | See sequence listing |
| MG108-2 crRNA | 260 | See sequence listing |
| MG108-2 tracrRNA | 236 | See sequence listing |

### Example 16—In Vitro Integration Activity Using Targeted Nuclease

In situ expression and protein sequence analyses indicated that some RNA guided effectors are active nucleases. They contain predicted endonuclease-associated domains (matching RuvC and HNH_endonuclease domains), and/or predicted HNH and RuvC catalytic residues. Candidate activity was tested with engineered single guide RNA sequences using the myTXTL system and in vitro transcribed RNA. Active proteins are identified as those that successfully cleave the library to yield a band around 170 bp in agarose gel electrophoresis.

### Example 17—Programmable DNA Integration

CAST activity was tested by combining five types of components in a single reaction: (1) a Cas effector protein expressed by my TXTL or PURExpress: (2) a target DNA fragment or plasmid containing the target sequence and PAM corresponding to the Cas enzyme: (3) a donor DNA fragment containing a marker or fragment of DNA flanked by the predicted LE and RE of the transposase system in a DNA fragment or plasmid: (4) any combination of additional transposase proteins predicted to be part of the array expressed using myTXTL or PURExpress; and (5) an engineered in vitro transcribed single guide RNA sequence. Active systems that successfully transposed the donor fragment were assayed by PCR amplification of the donor-target junction.

FIGS. 13A-13C shows example data demonstrating that the MG64-6 system comprising the MG64-6 effector, TnsB, TnsC, and TniQ proteins (SEQ ID NOs:30-33) using the predicted LE/RE donor sequences (SEQ ID NOs: 123-124) and in silico designed sgRNA (SEQ ID NO:201) is active. After performing the transposition reaction by combining all the MG64-6 components, PCR amplification of the junction showed that proper donor-target formation occurred and the transposition reaction was sg dependent. (FIG. 13A). Presence of amplified bands in PCR reactions #3 and #4 (spanning the LE/RE junctions when the LE/RE is inserted distal to the PAM, respectively) indicated that both orientations of the donor relative to the target are made: one where the LE is closer to the PAM, and one where the RE is closer to the PAM. While both transposition orientations were made, there was a preference for donor integration in the target where the LE is closer to the PAM, represented by strong band present for reactions #4 and #5 (which span the LE junction when it is inserted distal to the PAM and the RE junction when it is inserted proximal to the PAM, respectively).

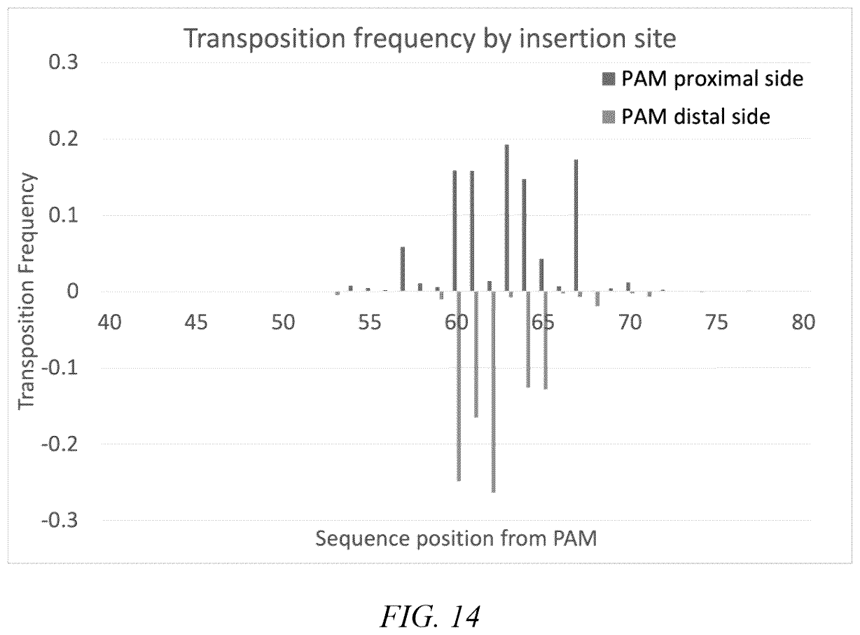

Sanger sequencing of the preferred orientation product was performed. Of the integrations that occur with the LE closer to the PAM, there was a clear degradation of the sequencing chromatogram signal from either the forward or reverse direction over the target/donor junction (FIG. 13C). This indicated that, of the products that are oriented with the LE closer to the PAM, integration occurred over a range of nucleotides, with the primary product of LE-closer-to-PAM products as a 61 bp integration from the PAM (FIG. 14). Sequencing that originated from the donor over the donor-target junction defined the composition of the essential outer bounds of the LE and RE sequences. Further investigation of the LE and RE domains will determine the inner limits of the LE and RE sequences and thus the minimal LE/RE that are essential for transposition. Sequencing of the RE on LE-closer-to-PAM products showed a 3 bp duplication downstream of the donor RE. This is in part due to the Tn7 transposase integration event that cleaves and ligates the donor fragment at a staggered cut site. A 3 bp duplication is smaller than the expected 5 bp of duplication from other Tn7 transposases.

Sanger sequencing of the PCR amplified product over the 8N library of the target plasmid also elucidated that the PAM preference of the MG64-6 effector as a nGTn/nGTt on the 5′ end of the spacer. NGS analysis of the PAM library target corroborated the nGTn motif preference at the 5′ end (FIG. 13B).

### Example 18—Integration Window Determination

PCR junctions of the PAM that were amplified in Example 17 above were indexed for NGS libraries and sequenced on a MiSeq with a V2 300 read kit. Reads were mapped and quantified using CRISPResso using an amplicon sequence of a putative transposition sequence with a 60 bp distance of integration from the PAM (guideseq=20 bp 3′ end of LE or RE, center of window=0, window size=20) Indel histogram was normalized to total indel reads detected, and frequencies were plotted relative to the 60 bp reference sequence (FIG. 14).

Both PCR reactions 5 (LE proximal to PAM, FIG. 13A) and PCR 4 (RE distal to PAM, FIG. 13B) were plotted on the sequence and distance from the PAM for MG64-6 (FIG. 14). Analysis of the integration window indicated that 95% of the integrations that occurred at the spacer PAM site are within a 10 bp window between 58 and 68 nucleotides away from the PAM. Differences in the integration distance between the distal and the proximal frequencies reflected the integration site duplication—a 3-5 base pair duplication as a result of staggered nuclease activity of the transposase upon integration.

### Example 19—Transposon End Verification Via Gel Shift

In order to verify the activity of TnsB on the predicted transposon end sequence, the RE of MG64-6 was amplified using FAM labeled oligos. MG64-6 TnsB protein was expressed using a cell free transcription/translation system and incubated with the RE FAM labeled product. After incubation for 30 minutes, binding was observed on a native 5% TBE gel (FIG. 15). Multiple bands of fluorescent product within the co-incubated lane (FIG. 15, lane 3) indicated a minimum of 3 TnsB binding sites.

### Example 20—Colony PCR Screen of Transposase Activity (Prophetic)

Transposition activity is assayed via a colony PCR screen. After transformation with the pDonor plasmids. E. coli are plated onto LB-agar containing ampicillin, chloramphenicol, and tetracycline. Select CFUs are added to a solution containing PCR reagents and primers that flank the insertion junction.

### Example 22—LE-RE Minimization (Prophetic)

Sequencing of the target-transposition junction helps to identify the terminal inverted repeats by identifying the outmost sequence from the donor plasmid that are incorporated into the target reaction. By performing repeat analysis of 14 bp with variability of 10%, short repeats contained within the terminal ends are identified; identifying the minimal sequences to be included in truncations of these that preserve the repeats while deleting superfluous sequence. Prediction and cloning is done in multiple iterations, with each interaction tested with in vitro transposition. Transposition is predicted to be active down to a LE region of 68 bp combined with a RE region of 96 bp.

### Example 23—Overhang Influence of Transposition (Prophetic)

In order to test whether superfluous sequence outside of the TnsB binding motifs are necessary for transposition, oligos designed for the TGTACA or TGTCGA motifs of both LE and RE are designed and synthesized with 0, 1, 2, 3, 5 and 10 bp extra base pairs. These synthesized oligos are used to generate donor PCR fragments with overhangs and tested for their ability to transpose into the target site.

### Example 24—CAST NLS Design (Prophetic)

Eukaryotic genome editing for therapeutic purposes is dependent on the import of editing enzymes into the nucleus. Small polypeptide stretches of larger proteins signal to cellular components for protein import across the nuclear membrane. Placement of these tags may require optimization, as these NLS tags need to provide import function while also maintaining function of the protein to which it is fused. In order to test functional orientations of the NLS to each of the components of the CAST complex, constructs fusing Nucleoplasmin NLS to the N-terminus and SV40 NLS to the C-terminus of each of the components of the MG CAST are synthesized. Proteins of these constructs are expressed in cell free in vitro transcription/translation reactions and tested for in vitro transposition activity with a complement set of untagged components. NLS-tagged constructs are assessed for maintenance of activity by PCR of the donor-target junction using PCR 4 (Assessing RE distal transpositions) and the cognate transposition event. PCR 5 (Assessing LE proximal transposition).

### Example 25—Cas12k and TniQ Protein Fusion Construct Design and Testing (Prophetic)

To simplify/minimize the expression of the protein components and facilitate delivery of these components into cells, fusion constructs between the Cas12k effector and the TniQ protein with various linkers, linker lengths, and domain boundaries are designed, synthesized, and tested. Both orientations of the TniQ fused to the Cas12k are designed and synthesized, a C-terminal fusion, Cas-TniQ, and an N terminal fusion, TniQ-Cas.

Two other linkers are also employed to fuse the effector and TniQ genes. P2A, a self-stopping translation sequence is active in a Cas-NLS-P2A-NLS-TniQ construct, and an MCV Internal Ribosome Entry Sequence (IRES) mRNA-based linker allows for independent translation of the two components in cells.

### Example 26—Intracellular Expression Coupled with In Vitro Transposition Testing (Prophetic)

To test the functionality of the NLS constructs in a physiologically relevant environment, constructs cloned with active NLS-tagged CAST components are integrated into K562 cells using lentiviral transduction. Briefly, constructs cloned into lentiviral transfer plasmids are transfected into 293T cells with envelope and packaging plasmids, and virus containing supernatant are harvested from the media after 72 hr incubation. Media containing virus is then incubated with K562 cell lines with 8 μg/mL of polybrene for 72 hrs, and transfected cells are then selected for integration in bulk using Puromycin at 1 μg/mL for 4 days. Cell lines undergoing selection are harvested at the end of 4 days, and differentially lysed for nuclear and cytoplasmic fractions. Subsequent fractions are then tested for transposition capability with a complementary set of in vitro expressed components.

Both NLS-TnsB and TnsB-NLS are tested by cell fractionation and in vitro transposition, and transposition is detected across both cytoplasmic and nuclear fractions

Cas12k fusions in the cell are similarly fractionated and tested for transposition. Cas-NLS Cas-NLS-P2A-NLS-TniQ are transduced into cells, fractionated, and tested in vitro for subcellular activity. Cas-NLS-P2A-NLS-TniQ is able to transpose in the cytoplasm with the addition of single guide to the reaction. By supplementing holo Cas protein (+sgRNA) or additional TniQ with sgRNA, we are able to complement the Cas-NLS-P2A-NLS-TniQ construct in the nuclear fraction.

Systems of the present disclosure may be used for various applications, such as, for example, nucleic acid editing (e.g., gene editing) or binding to a nucleic acid molecule (e.g., sequence-specific binding). Such systems may be used, for example, for remediating (e.g., removing or replacing) a genetically inherited mutation that may cause a disease in a subject; inactivating a gene in order to ascertain its function in a cell; as a diagnostic tool to detect disease-causing genetic elements (e.g. via cleavage of reverse-transcribed viral RNA or an amplified DNA sequence encoding a disease-causing mutation); as deactivated enzymes in combination with a probe to target and detect a specific nucleotide sequence (e.g. sequence encoding antibiotic resistance int bacteria): to render viruses inactive or incapable of infecting host cells by targeting viral genomes: to add genes or amend metabolic pathways to engineer organisms to produce valuable small molecules, macromolecules, or secondary metabolites: to establish a gene drive element for evolutionary selection, and/or to detect cell perturbations by foreign small molecules and nucleotides as a biosensor.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

1.-141. (canceled)

142. An engineered nuclease system comprising:
an endonuclease comprising a RuvC domain, wherein said endonuclease is a Class II, type V endonuclease having at least 80% identity to any one of SEQ ID NOs: 30, 22, 26, 34, 55-89, 104, or 147; and
an engineered guide ribonucleic acid (RNA), wherein said engineered guide RNA is configured to form a complex with said endonuclease and said engineered guide RNA comprises a spacer sequence configured to hybridize to a target nucleic acid sequence.

143. The engineered nuclease system of claim 142, wherein said endonuclease comprises a sequence having at least 90% sequence identity to SEQ ID NO: 30.

144. The engineered nuclease system of claim 142, wherein said endonuclease comprises SEQ ID NO: 30.

145. The engineered nuclease system of claim 142, wherein said engineered guide RNA comprises a sequence comprising at least about 46-80 consecutive nucleotides having at least 80% sequence identity to SEQ ID NO: 92.

146. The engineered nuclease system of claim 142, wherein said engineered guide RNA comprises a sequence having at least 80% sequence identity to non-degenerate nucleotides of SEQ ID NO: 111 or 257.

147. The engineered nuclease system of claim 142, wherein said endonuclease is configured to bind to a protospacer adjacent motif (PAM) sequence, wherein said PAM sequence comprises 5′-nGTn-3′ or 5′-nGTt-3′.

148. A system for transposing a cargo nucleotide sequence to a target nucleic acid site comprising:
a first double-stranded nucleic acid comprising said cargo nucleotide sequence configured to interact with a Tn7 type transposase complex;
a Cas effector complex comprising a class II, type V Cas effector and an engineered guide polynucleotide configured to hybridize to said target nucleic acid site; and
said Tn7 type transposase complex, wherein said Tn7 type transposase complex is configured to bind said Cas effector complex, wherein said Tn7 type transposase complex comprises a TnsB component, a TnsC component, and a TniQ component, wherein:
(a) said Class II, type V Cas effector comprises a polypeptide comprising a sequence having at least 80% sequence identity to any one of SEQ ID NOs: 30, 22, 26, 34, 55-89, 104, or 147; or
(b) said TnsB component, said TnsC component, or said TniQ component comprises a sequence having at least 80% sequence identity to any one of SEQ ID NOs: 31-33, 23-25, 27-29, 35-37, 101-103, 105-107, or 148-150.

149. The system of claim 148, wherein said cargo nucleotide sequence is flanked by a left-hand transposase recognition sequence and a right-hand transposase recognition sequence.

150. The system of claim 148, wherein said Class II, type V Cas effector comprises a polypeptide comprising a sequence having at least 90% sequence identity to SEQ ID NO: 30.

151. The system of claim 148, wherein said Class II, type V Cas effector comprises a polypeptide comprising SEQ ID NO: 30.

152. The system of claim 148, wherein said TnsB component comprises a sequence having at least 90% sequence identity to SEQ ID NO: 31.

153. The system of claim 148, wherein said TnsC component comprises a sequence having at least 90% sequence identity to SEQ ID NO: 32.

154. The system of claim 148, wherein said TniQ component comprises a sequence having at least 90% sequence identity to SEQ ID NO: 33.

155. The system of claim 148, wherein said engineered guide polynucleotide comprises a sequence comprising at least about 46-80 consecutive nucleotides having at least 80% identity to SEQ ID NO: 92.

156. The system of claim 148, wherein said engineered guide polynucleotide comprises a sequence having at least 80% sequence identity to SEQ ID NO: 111 or 257.

157. The system of claim 148, wherein said Cas effector complex is configured to bind to a protospacer adjacent motif (PAM) sequence, wherein said PAM sequence comprises 5′-nGTn-3′ or 5′-nGTt-3′.

158. The system of claim 148, wherein said Tn7 type transposase complex binds non-covalently to said Cas effector complex.

159. The system of claim 148, wherein said Tn7 type transposase complex is covalently linked to said Cas effector complex.

160. The system of claim 149, wherein said left-hand transposase recognition sequence and a right-hand transposase recognition sequence comprises a sequence having at least 80% identity to SEQ ID NO: 123.

161. The system of claim 149, wherein said right-hand transposase recognition sequence and a right-hand transposase recognition sequence comprises a sequence having at least 80% identity to SEQ ID NO: 124.


SEQUENCE LISTING (261 sequences)

SEQ ID NO: 1 - protein, 1424 aa, unidentified
  REGION 1..1424: note = Description of Unknown: MG36 effector sequence
  REGION 1..1424: note = MG36-5 effector
MKRILGLDLGTNSIGWALVNEAETDEEVSSIIRLGVRVNPLTVDETQNFEKGKSITTNAERTLKRSMRRNLQRYKLRRDALVEFLKELGFITDETILSEQGNRTTFETCRLRAKAAEEEISLEELSRVLLMINKKRGYKSSRKVKKEEDGVLIDGMEIAKKLYEEDLTPGQLCSQLLESGKKVLPDFYRSDLQEELDRIWSVQKQFHPDVFCDAAKEEIKGKNRSQTWAILANYFVWKEEVVGWNDREAKNETIEKECKLVGLKRTTKGYELKKENYRWRAQALTEQLGLEEIAVVLQEINGQINASSGYLGAISDRSKVLYFNHQTVGQYQMAELDKNPHASLRNMVFYRQDYLDEFDRIWEKQAEFHKELTAELKKEIRDMIIFYQRRLKSQKGLISFCEFERLEIVVEKDGKKQTKVIGCKVIPRSHPLFQEFKVWQTLNDIKVLGREKKRNALSASESRALYPEEKEILARELAIKENMKKADVLKLLFENPQELDLNFKQIDGNRTGFSLFSAYSKMIEKYGYEPLDFKKPADEIIGQLRTIFTDLGWNTNLFTIDLTKEGKELEMQPYFRLWHLLYSFEGDNTSTGNGKLIEKIMQLCGVEKEYAVELASLSFQDDYGSLSAKAIKRILPYLKEGNLYDVACEYAGYRHSKSSLTKEEIENKVLKGKLEVLPKNSLRNPVVEKILSQMVNVINTIIDTYGKPDEIRVELARELKKSAKEREELTKAIAKSTREHEEIRKLLQDEFGMMNVSRNDIIRYKLYEELKDNGYKTLYSNQYIPKEKIFSKEIDIEHIIPQSRLFDDSLSNKTLEYKAINIEKGNKTAYDFVKEKYGEEGLQQYINRCESLFNDKKAKLRKLKMEQKDIPDGFIDRDLRNTQYIAKKALAMLNEICRRVVATTGSITDQLREDWQLVDVMKELNLPKYEALGFVETYEDKDGRKIKRIKDWTKRNDHRHHAMDALTVAFTKDAFIQYFNNKNAAQDSGNRNHTNIIGIKTRYFDKGRALPPIPLEQFRIEAKQHLEKLLVSIKAKNKVVTVNVNRTKKRKGENTKIQQTPRGQLHLETVYGSHKQYVTKIEKVNASFDAAKIATVSKRAYRNALLKRLETFGNDPKKAFTGKNALEKNPLYADKYQTIRVPEKVQTVEFETIYTIRKPVDPALNVDKVVDVKVRAILERRLKEYGGDPKKAFVNFWENPIWLNKEKGISIKRVSIRGINNAQSIHVKKDKNGNPIWDENGKQIPVDFVNTGNNHHVAIYRKPVLDKKGQVTFDEDGNLMYELDEVVVPFFEAVTRANLGLPIIDKDYRKSEGWQFLFSMKQNEYFVFPNEKTGFNPKEVDLLNPDNYAMISPNLFRVQTMSKVMYGNNVVRDYKFRHHLETTVKDMKELKDIAYKQYKTLSFGNSVVKIRINHIGQIVSVGEY

SEQ ID NO: 2 - protein, 264 aa, unidentified
  REGION 1..264: note = Description of Unknown: MG36 transposition protein sequence
  REGION 1..264: note = MG36-5-B1 transposition protein
MKADVSERQRVKLLADMIGYYRLCCPRIGGFKLFHLLEKDLGHAVTLGRDSFLKVYESKGFKLNPNKRRRTTDSNHVYKRYPNLIKGKDARYSNHIWVSDITYVWILGDVLYLHLVTDAYSHAVLGWCLSDSLSASHTTEALRMAIRIAGGGNLCGTIHHSDRGSQYASEAYVSCLMEHHIRISMTEGYEPTDNAMAERQNGIFKVEWIYEQEMYRDKEQAINEINRMIDFYNNRRPHMSIGMECPMEVYKGKLPGKNLWRKRP

SEQ ID NO: 3 - protein, 126 aa, unidentified
  REGION 1..126: note = Description of Unknown: MG36 transposition protein sequence
  REGION 1..126: note = MG36-5-B2 transposition protein
MIKRKPKRVFTEPFKLSVLREYYSSGMSKYAITKKYSLSPPCIYRWLKEYPVGSDQLPLPSETKERLQMVPKQSDLTDMESLQKRIEELRRSLELEKMRSRAFEKMIEIAEEEEGISIFRKDGAKQ

SEQ ID NO: 4 - protein, 286 aa, unidentified
  REGION 1..286: note = Description of Unknown: MG36 transposition protein sequence
  REGION 1..286: note = MG36-5-T1 transposition protein
MARPNKEGLDYFPFDVDFFSDEKIGSISGEFGIKGEITAIKLLCAIYRNGYFILWNDALKMSLLRGLPGISLELLEQIVTRLVRWGFFEQTLFSTVSVLTSKGIQERYFKAIKRRKDSSNYPYLLVNVDNNKVNVSNNDINVNTNPIKERKGNKNRESLNTRETLFDNFKNELLRDEEWRRYACQISGLSVAFNDLIPGELDNFLAWMVSTGEGDTLKTIDDVKRRFTYWWQGTGLRAYNQRYGGTRKETFGGYTSHAGAYGKREAPAKTGVQPSEEARKDYTERF

SEQ ID NO: 5 - protein, 203 aa, unidentified
  REGION 1..203: note = Description of Unknown: MG36 transposition protein sequence
  REGION 1..203: note = MG36-5-C transposition protein
MIKALGCNYLGIERRQFETDRGNDKVLRFLLYYFNDCPLAESVFPEENYKLHKNLLIVGDPGTGKTLMMQIFADYLKLTDNPKRFVNLSVTQMMNYYKIHGHIDRFTYNEEAGKGSMEGNPFDICLNDIGLETENQKSYGTSLNSVIDEFLYARYEIYQSHQKKYHITSNLSVTDFKNRFGTRLVDRFKSFNVIILNGESRRR

SEQ ID NO: 6 - protein, 724 aa, unidentified
  REGION 1..724: note = Description of Unknown: MG39 effector sequence
  REGION 1..724: note = MG39-1 effector
VKIIKRSGAENTFDKEKIENAVAKANITVEEKDRLSEGEIGEIAQNIEDKCSEMNRAMDVETIQDWVEADIMRHGKYTVAKHYITYRYERSIVRQANTTDKQILSLLNFENEEVKQENSNKNPTVNSVQRDYMAGEVSKDITRRFLLPDDIVEAHEKGLIHFHDADYFAQHMHNCCLVNLEDMLQNGTVISEVMIEKPHSFSTACNIATQSIAQIASSQYGGQSITLSHLAPFVQISRDKYRREVKKEFAELNIPADEDTINKVAEMRVKAEIVQGVQMIQYQVITLMTTNGQAPFVTVFMYLDEVPEGQTRDDLAAIIEEMLRQRIQGVKNEKGVYITPAFPKLIYVLEEDNIREGSKYWELTKLAAKCTAKRMVPDYISEKKMKELKVDKNGNGQCYPCMGCRSFLTTYLDENGKPKYYGRFNQGVVTINLVDVACSSYKDMDKFWLIFDERLELCRRALMLRHERLKGTPSDVAPILWQNGALARLKKGETIDKLLFGGYSTISLGYAGLCECVRYMTGKSHTDPSATPFALEVMQHLNDACAKWRAETNIDFSLYGTPLESTTYKFARCLQKRFGVIEGVTDRNYITNSYHIHVTENIDAFDKLTFESQFQALSPGGAISYVEVPNMQNNIEAVLAVMQHIYDNIMYAELNTKSDYCQKCGFDGEIKIVEDDGKLVWECPNCGNRDQNTLNVARRTCGYIGTQFWNQGRTQEIKERVLHL

SEQ ID NO: 7 - protein, 214 aa, unidentified
  REGION 1..214: note = Description of Unknown: MG39 transposition protein sequence
  REGION 1..214: note = MG39-1-A transposition protein
MSKRISRATKLARGQGSGEGAKYKSYVMTNEFNSLGTTSVIKDWKTGRGVHCLSQAEALWFYILRWDDNNIDIREQYPLDRNITSRIADKYGFKHPGNSDHIMTTDFLVTKKNNKLHAYSVKPDRDLSKRTLEILCIEKLYWEHNNIEFDMLFKEDVNTILASNIRLVTEYYDESRVFDRYSNIRHNIATKKIRCDMEHKILTNDDLDRIWGEY

SEQ ID NO: 8 - protein, 716 aa, unidentified
  REGION 1..716: note = Description of Unknown: MG39 transposition protein sequence
  REGION 1..716: note = MG39-1-B transposition protein
MTILIEYGVNTEMNDYVLAIGDILYDKDNQNSYRIISLIDDHLILCEMETTKLELQQIKYTIIADLVLSNKIEIKKDQALVYDIDQLSESVRNRYILKVHIMNDVKIAYGPSYLGLMGKSSKIELQKILAKYNYPISSFWRMCTTYFQSGMKNYSLINAKSFKSNEVKTYTYKARPGAKSTYNLDNVVKPNEYVIYFEEALNEYKAGREKTLKNAFSRMNILHFTQAEIIDGVATRLLLPECQRPTYKQFYYYAQKHLTKEEKDLIKTSAAEQRNNKRLLISDSLKDVYGPADMVEIDACEADVSLVSELDPDQAIGRPIVYFMIDVYSRIILAVSVAFDNNSILGITNLFLNLADDKQEYCKKYGIEFNDKRLWPSGVIPKRIRVDRGSEFKSYEFDRICNELGIEKQIVSGASGSLKGVVEQAFHQMHAKQNVHLENHGLIEKRYDSLHHKEASLTIHDYTRMVINFVLAHNQQHLETYPLTKEMIEKNIAPVPAILWEYGSKKYGMPQPIPVLEQYLFSLMTPIKAKISKRGISYKGLWYFAPNDKRLMSEMYAAGTRRMPFEVRMDMRDVGAIYYIRNSKLVKIPLNVLITGNSDYKGLTMKQYEEYYSAKKKMQAKGRIDNEKIDTAVYANNESIVKSAKKNVHSRTKDIRSSREIDKQKVSYEGKISARLENKDETSKNISSDEQKENGVTEYRDYASFEEALQDFYDNN

SEQ ID NO: 9 - protein, 534 aa, unidentified
  REGION 1..534: note = Description of Unknown: MG39 transposition protein sequence
  REGION 1..534: note = MG39-1-C transposition protein
MAKYNYVTCEEVVSQYDDYDLKEQRIVPARYVEAKLAIDTGNPYIEALPYPRTGRNIISSYSQTMADFDYDKIKSMSTIDKILQIRSLRSIRFPLPFHAELELSFYNALITSYRSRHILHSDNDKVSYSVENQEYAASNILVGDSSASTDAGFSLIGYSGCGKSSAIQMLVSYYPQVIMHTTENGEYFPQITYLVVNCIPNSNFSALYDGIGDAIDKALGNIKPIYSAEIMKIRTLGAKAERIREYVEKFAIGIIIFDEIQLIDFSHTRENSFDSLLTLSNRTKVATAIVGTEDAKAKMFKTLRTARRVGNVINGNMYCMDKKFFYFLVNELFRYQWFDKPVTVSEEITDALYDVTKGIVDQLIGIYIYMNIDYLNKKKKPEINSKYIYAVTNKHFAGLRDVLENLDSDFNNELMQEINDNAKLELDKIEDEAKQLKEAEKILNNNSSEEKVQIKNVVANITAIYDEYTPTQIEDAFKKVIRKKSSVGKAEREISKLVIEQLTKQQRTRSDLCKKNTPSVIQMQNFLGIDKEDK

SEQ ID NO: 10 - protein, 641 aa, unidentified
  REGION 1..641: note = Description of Unknown: MG39 transposition protein sequence
  REGION 1..641: note = MG39-1-Q transposition protein
MPVIPVYINPYPDELMYSWIHRLAKKNGLPITTFADSYLNKFNSKIGSLEYDIRYGLLCLNESFFIQKDLKEMFLSMSIFPFEAMFFSVGQQTRYVNNMFRKPDPLNAPVNTMIREIHICPQCIENDIEIFGEPYIHRAHQLSGVCTCHKHKAPLYKYTGIKGHECEYDLSHYTELEIKDIAIENEYTDYAQALFNSNTNCNIADLKHMIYNKLKELGHKATNGYEDFISAFNDSKLVTLFDADLKNFLLVSIISTQSTSARSMLPLLMYLFPNVQEIIHRFENAPPIIQKYHCAECGKNFYATPTSLTEGWGCTYCDANKSIDERYKKLIDFAGKGNYEPLEPFRSLNIKSKIHHKICGETIQIKPRKFIFDHVRCICESLLNEWDVRQKLEKFEDYEFISYDSESSKITMRSKACGHVFSCRFHKFIKYPSCRVCRPKNMTTELYTERVYDLVGDDYTVLSEFVDQRTKIAIKHNKCGGIQEYKPSAFLDGQRCNACNSLIVKKANDSWEKGYALLCEYKEECGTANIPKRDHYKGVFLGNWLQSQRDKYKAGKLTRSQEDALVSLGITLDPLAAEWERRYEQYKRYIQQNNGSSDITKRTIFEGEKLGVWVVLQRRNYNIGKLSEERYKKLCDINMKF

SEQ ID NO: 11 - RNA, 118 nt, synthetic construct
  misc_feature 1..118: note = Description of Artificial Sequence: Synthetic polynucleotide
  misc_feature 1..118: note = MG36-5 effector tested sgRNA sequence
  source 1..118: mol_type = other RNA
  misc_difference 1..24: note = any nucleotide
nnnnnnnnnnnnnnnnnnnnnnnngttgtgatttgctttcagaaaatgaagcaaatcacaataaggattattccgttgtgaaaacattaggttccctcgcccttctgcgggggatttt

SEQ ID NO: 12 - RNA, 80 nt, synthetic construct
  misc_feature 1..80: note = Description of Artificial Sequence: Synthetic oligonucleotide
  misc_feature 1..80: note = MG36-5 effector tracrRNA sequence
  source 1..80: mol_type = other RNA
tactaaaatgaagcaaatcacaataaggattattccgttgtgaaaacattaggttccctcgcccttctgcgggggatttt

SEQ ID NO: 13 - RNA, 46 nt, synthetic construct
  misc_feature 1..46: note = Description of Artificial Sequence: Synthetic oligonucleotide
  misc_feature 1..46: note = MG39-1 effector tracrRNA sequence 1
  source 1..46: mol_type = other RNA
tttttgtaaaatcattttgtaaaattacaattcataatgagatatg

SEQ ID NO: 14 - RNA, 66 nt, synthetic construct
  misc_feature 1..66: note = Description of Artificial Sequence: Synthetic oligonucleotide
  misc_feature 1..66: note = MG39-1 effector tracrRNA sequence 2
  source 1..66: mol_type = other RNA
tttttgtaaaatcattttgtaaaattacaattcataatgagatatgacaattcataatgagatatt

SEQ ID NO: 15 - RNA, 109 nt, synthetic construct
  misc_feature 1..109: note = Description of Artificial Sequence: Synthetic polynucleotide
  misc_feature 1..109: note = MG39-1 effector tracrRNA sequence 3
  source 1..109: mol_type = other RNA
aaaatataggatattaatgtcagagatatctcattataattgtaattttgttttgtgtacaagctgttaatatctcattatgaattgtcatatctcattatgaattgta

SEQ ID NO: 16 - RNA, 88 nt, synthetic construct
  misc_feature 1..88: note = Description of Artificial Sequence: Synthetic oligonucleotide
  misc_feature 1..88: note = MG39-1 effector tracrRNA sequence 4
  source 1..88: mol_type = other RNA
aaaatataggatattaatgtcagagatatctcattataattgtaattttgttttgtgtacaagctgttaatatctcattatgaattgt

SEQ ID NO: 17 - DNA, 36 bp, synthetic construct
  misc_feature 1..36: note = Description of Artificial Sequence: Synthetic oligonucleotide
  misc_feature 1..36: note = putative MG36-5 transposon end LE1
  source 1..36: mol_type = other DNA
ttaattgaaaattgaaaattgagaattgaaaattaa

SEQ ID NO: 18 - DNA, 36 bp, synthetic construct
  misc_feature 1..36: note = Description of Artificial Sequence: Synthetic oligonucleotide
  misc_feature 1..36: note = putative MG36-5 transposon end LE2
  source 1..36: mol_type = other DNA
tgagaattgaaaattgagaattgggaattgagaatt

SEQ ID NO: 19 - DNA, 28 bp, synthetic construct
  misc_feature 1..28: note = Description of Artificial Sequence: Synthetic oligonucleotide
  misc_feature 1..28: note = putative MG36-5 transposon end RE1
  source 1..28: mol_type = other DNA
ttaattttcaattctcaattttcaatta

SEQ ID NO: 20 - DNA, 36 bp, synthetic construct
  misc_feature 1..36: note = Description of Artificial Sequence: Synthetic oligonucleotide
  misc_feature 1..36: note = putative MG39-1 transposon end LE1
  source 1..36: mol_type = other DNA
aaaagtattgacaaattgacaaaatggtggtataat

SEQ ID NO: 21 - DNA, 36 bp, synthetic construct
  misc_feature 1..36: note = Description of Artificial Sequence: Synthetic oligonucleotide
  misc_feature 1..36: note = putative MG39-1 transposon end RE1
  source 1..36: mol_type = other DNA
attataacctatatttttgattttgtcaatagtttt

SEQ ID NO: 22 - protein, 637 aa, unidentified
  REGION 1..637: note = Description of Unknown: MG64 effector sequence
  REGION 1..637: note = MG64-2 effector
MSQITIQCQLVASASTRQQLWLLMTQKNTPLINELLQQVGQHPEFETWRQKGKLQAGIVKALCQPLKADPRFMGQPARFYASAIAVVDYIYRSWLALQKRLQYQLEGQTRWYQMLRSDAELIELYGGSLETLRSKAAEILAQFAPESASVNPQPTKGKKSKKRKNSSNNPNLSTALFEAYRQTEDILSSCAINYLLKNGCKVSEKEEDPEKFAKRRRSVEIRIERLKEKLASRMPKGRDLTDEKWLETLMVASTTVPSSEFQAKSWQDNLLRKSSAIPFPVAYETNEDMTWFKNSKGRICVKFNGLSEQTFEVYCDSRQLYWFQRFLEDQQIKRNSKDQHSSSLFTLRSGRIAWSEGEGKGDPWNIHRLTLYCCVDTRLWTSEGTEQVRHEKADEITQIITKTKQKTDLNEKQQAFIKRKTSTLARINNPFPRPSKPVYQGHSHILVGVSLGLDKPATLAVIDAIANKVITYRSIRQLLGDNYQLLNRQRQQQHQNAHKRQTAQRQDAPHQFSESELGQYIDRLLAKAIVAVAKTYQAGSIVLPQLGDVRESIESEIKARAEQKCPDLVEVQKQYAKQYRSSIHRWSYARLIDSIKSQASQVGIFIEEGKQPVRGSPQDKAKDLAIAVYHSRLNTKS

SEQ ID NO: 23 - protein, 560 aa, unidentified
  REGION 1..560: note = Description of Unknown: MG64 transposition protein sequence
  REGION 1..560: note = MG64-2-B transposition protein
MQDDRESEVPIPAEVNEIVTDFSEDAKLMQEVIQSLLEPCDRITYGQRQREAAAKLGKSVRTIRRLVKKWETEGLSALQPNKRTDKGKHRIDQEWQEFIIKTYKEGNKGSKRITPQQVAVRVAAKAADLGQEKYPSYRTVYRVLQPIIEKQEKTQSVRSRGWRGSRLSVKTRDGQDLSVEYSNHVWQCDHTRADILLVDQDGQLLGRPWLTTVIDTYSRCIIGINLGYDAPSAYVVALALRHAILPKQYSSEYKLHCQWGTYGKPEHFYTDGGKDFRSNHLQQIGVQLGFVCHLRDRPSEGGIVERPFGTFNTDLFSTLPGYTGSNVQERPEEAEKEASLTLRELEQLLVRYIVDKYNQSIDARMGDQTRFQRWEAGLIAAPNLISERDLDICLMKQTRRTIYRGGYLQFENLTYRGDYLEGYSGESVVLRYDPKDITTILVYRKEGDKEVFLARAYEQDLQTEQLSFDEVKAISRKLREAKKAVNSRSILAEVRDRETFLTQKKSKKQRQKSEQAQIHRKKELFPIEAEATEFESPVNELETEIIEVFDYEQMREDYGF

SEQ ID NO: 24 - protein, 287 aa, unidentified
  REGION 1..287: note = Description of Unknown: MG64 transposition protein sequence
  REGION 1..287: note = MG64-2-C transposition protein
MMNQEKEAKAIAQKLGNIPLNDEKIQAEIQRLNRKNFVPLEQVKALHDWLESKRQARQCCRVIGESRTGKTMACNAYRLRNKAIQSLGQPPTVPVVYIQIPQECTPKELFSVLLEHLNHQMTKGTTAEMRNRTLRVLKACRVEMLIIDEADRLKPKTFADVRDIFDNLEISVVLVGTVRLEKVMTDDEQVCNRFSACYRYGKLSVEEFKKTVNIWENQVLKLPVSSNLTQSKMLDILKDKTQCYIGLMDMILRDAAIRALNKGMPKIDLETLKEVTNEYTAPPKRSK

SEQ ID NO: 25 - protein, 168 aa, unidentified
  REGION 1..168: note = Description of Unknown: MG64 transposition protein sequence
  REGION 1..168: note = MG64-2-Q transposition protein
MKASDIQPWLFRVEPFEGESISHFLGRFQRANELTPSGLAKAAGLGGAIARWEKFRFNPPPSPQQLEALALVVEVEAERLVQMLPPAGVGMKMEPIRLCGACYAEVPCHKIEWQFKTTQGCDRHQLSLLSECPNCGARYKVPALWPDGWCSRCFLPFADMVKWQKLIV

SEQ ID NO: 26 - protein, 637 aa, unidentified
  REGION 1..637: note = Description of Unknown: MG64 effector sequence
  REGION 1..637: note = MG64-4 effector
MSQITIQCRLVCCESTRASLWKLMAEKNTPLTNELLHQVGHHCEFESWRKKGKIPSGIVKELCQPLKTDPRFTGQPGRFYTSAIALVSYIYKSWLVLMKRSHYKLEGKTRWLEMLSSDAELVERCGISVDGIRARASEILAQLALPDSADTGQVKGRKAKKSKKFQQQQDRDRNLSRTLFEAYRDTEDILTRCAICYLLKNGCQVSDREENSEKFTQRRRQVEIQIERLKEKLAARIPKGRDLTDAKWLETLFLATSHAPESEAEAKLWQDCLLRQPSSVPFPVAYETSEDMTWFKNHKGRISVKFNGLSEHTFEVYCDSRQLHWFERFLEDQQIKRNNQNQYSSSLFTLRSSRIAWQEGKGKGEPWNFNHLTLYCSVDTRLWTAEGTQLVIEEKVEEIVKNITNTKDKGNLNEKQLAHIQRKNSTLARITNPFPRPSKSIYRGQSHILVGVSLGLEKPATVAVVDATTSNVIAYRSIKQLLGDNYRLLNRQRQQKQVLSHQRHLAQRLDALNSLGESELGQYVDRLLAKEIVAIAQTYCAGSIVLPSLGDMREQVQSEIQAKAEQKSDLIEVQQKYAKQYRASVHQWSYGRLISSIQSQAKKAGIAIEEAKQPIRGSPQQKAKELVIAAYNARKTS

SEQ ID NO: 27 - protein, 563 aa, unidentified
  REGION 1..563: note = Description of Unknown: MG64 transposition protein sequence
  REGION 1..563: note = MG64-4-B transposition protein
MSKLSEDNHIDNQKPAVGEIVPEIADDNKQLLEVIQKLLEPCDRITYGQRQREAAAQLGKSVRTVRRLVKKWEEEGLAALSQTTRADKGKHRIEQDWQDFIIKTYKEGNKGSKRITPKQVAVRVQAKAAELGQDRYPSYRTVYRVLQPIIERQEQQASVRSRGWRGSRLSVKTRDGKDLSVEYSNHVWQCDHTRVDVLLVDRNGAILSRPWLTTVVDTYSRCIMGFNLGYDAPSSQVVALALRHAILPKQYDWEYQLDCDWGTYGKPEHFYTDGGKDFRSNHLQQIGVQLGFASHLRDRPSEGGIVERPFGTFNTEFFSTLPGYTGSNVQERPEQAEKEARLTLRELERLLVRYIVDKYNQSIDARLGDQTRFQRWEAGLIAAPNPIAERDLDICLMKQTRRSIYRGGYLQFENLTYRGENLAGYAGESVVLRYDPRDITTVLVYRQEAGKEVFLARAFAQDLETEQMSLDEAKASSRKLRETGKTISNRSILAEVRDRETFLTQKKTKKERQKAEQAEVKRAKQPFSVEREEEIEAASIPNQPEPEMPDVFDYEQMREDYGF

SEQ ID NO: 28 - protein, 278 aa, unidentified
  REGION 1..278: note = Description of Unknown: MG64 transposition protein sequence
  REGION 1..278: note = MG64-4-C transposition protein
MSLKDAQAIAQQLGNIQPNDARLQTEIQRLNGKSFVPLEQVKILHDWLDGKRQARQGCRVVGESRTGKTIACDAYRLRHKPIQELGKPPIVPVAYILVPPDCGSKDLFGLIVEHLKYQMTKGTVAEIRERTRRVLKGCGVEMLIIDEADRLKPNTFRDVRDIGENLGITVVLVGTDRLDAVIKKDPQVYNRFRACHRFGNLSGDDFKRTVEIWEKKVLQLPVASNLSGKTMLKTLGEATGGYIGLLDMILREAAIRALKKGLQKIDLETLKEVAGEYR

SEQ ID NO: 29 - protein, 167 aa, unidentified
  REGION 1..167: note = Description of Unknown: MG64 transposition protein sequence
  REGION 1..167: note = MG64-4-Q transposition protein
MEASDIQPWLFRAEPYEGESISHYLGRFRRANDLTPTGLGKATGLGGAIARWEKFRFNPPPSRKQLEALAVVVGVEADRLAQMIPPAGVGMKLEPIRLCAACYAQAPYHRIEWQFKVTAGCDHHQLRLLSECPNCKARFKIPALWVDGWCQRCFTKFEEMTSNQAAL

SEQ ID NO: 30 - protein, 632 aa, unidentified
  REGION 1..632: note = Description of Unknown: MG64 effector sequence
  REGION 1..632: note = MG64-6 effector
MSQITIQCRLIAKESSRQALWRLMAELNTPLINDILNQIANHPDFETWREKGKLPAGIVKQLSDSLKTDPRYIGQPGRFYTSAITLISYIYKSWFKVQQRLQQRLVGQTRWLGILKSDEELVAESDRTLEEIRAKAIGLLASLTPENPSPEPKPAKKTKKAKTSTNKPLLGILFDNYEKTEDILTHAAICYLLKNGCKIPTKLEEPQEFAKKRRKAEIKVERLQEQLNSRKPKGRDLTGEKWLQTLITASTTAPENEAQAKSWQNILLTKSKSIPFPVAYETNEDLTWSKNDKGRLCVHFNGLGEHEFEIYCDQRQLKWLQRFYEDQETKRASKDQHSSALFTLRSGRIGWQEGKGKGEPWNIHRLNLFCTIDTRFWTAEGTEQVRQEKATEIAQTITKMEQKGDLNDKQQAFIHRKHSTLARINNPFPRPSQPLYQGKSHILIGIAMGLENPATAAIINGTTGEALAYRSIKQLLGDNYQLLTRQQKQKQRQSHQRHKAQKNAAPNQFGESELGEYVDRLLAKAIVALAKTYQAGSIVVPELGDMRELVQAEVKAKAEAKIPGCIEAQEKYAKQYRVNTHQWSYGRLIDNIQAQASKIGIVIEQGQQPIRGSPQEKAKEMALLAYHSRSKS

SEQ ID NO: 31 - protein, 578 aa, unidentified
  REGION 1..578: note = Description of Unknown: MG64 transposition protein sequence
  REGION 1..578: note = MG64-6-B transposition protein
MKKLFAQDVNIDTEVISNQIPTSDPSQSNLIASELPEEARPKLEVIQSLLEPCDRVTYGERLREGAEKLGLSVRSVQRLFKKYQEKGLIALLSGSRTDKGEHRISELWQNFIVKTYQEGNKGSKRMSPKQVALKVQAKAGAIADDNPPSYRTVLRVLKPILEKQEKAKSIRSPGWRGSTLSVKTRDGDDLDISYSNQVWQCDHTRADVLLVDQHGKLLVRPWLTTVIDSYSRCIMGINLGFDAPSSQVVALALRHAILPKRYGTEYKLNCDWGTYGTPEYLFTDGGKDFRSNHLAEIGLQLGFVCKLRDRPSEGGIVERPFKTLNQSLFSTLPGYTGSNVQERPEDAEKDAQLTLRDLEQLIVRFIVDRYNQSIDARMGDQTRYQRWEAGLQKEPDVISERDLDICLMKMSRRTVQRGGHLQFENVMYLGEYLAGYAGEVVSFRYDPRDITTIWVYRQENDREVFLTRAHAQGLETEQLSVDDAKASAKRLRAAGKTISNQSILQETIEREVLAERTKSRKHRQKEEQSYKRSPSAAVMVEVESEQLEIESSNEANANSVSADIEVWDYDEMREGLGW

SEQ ID NO: 32 - protein, 284 aa, unidentified
  REGION 1..284: note = Description of Unknown: MG64 transposition protein sequence
  REGION 1..284: note = MG64-6-C transposition protein
MIKENSSQEKPASQIAEELGDFKVDSQLLQIEIARLNKKSIVPLEHIKDLHDWLDEKRKARQSCRLVGESRTGKTVACEAYTFRNKPKQEGKQAPTVPVVYIMPPAKCGAKELFREIIEYLKYRAVRGTVADFRSRAMEVLKGCEVEMIIIDEADRLKPETFSDVRDINDKLGIAVVLVGTDRLDAVIKRDEQVYNRFRASRRFGKLTGEDFKRTVEIWEDKVLKMPVASNLTNKEMLKILLKATEGYIGRLDEILREAAIKSLSRGFRKVEKAVLQEVAREYS

SEQ ID NO: 33 - protein, 167 aa, unidentified
  REGION 1..167: note = Description of Unknown: MG64 transposition protein sequence
  REGION 1..167: note = MG64-6-Q transposition protein
MTENDIQPWLFAIAPLPGESLSHFLGRFRRRNHLTPSSLGQIAKIGAVVARWERFHFNPYPTQQEFEALAEVVGVEVERVWEMLPPMGEGMKCEPIRLCCACYAESPCHRIEWQFKSVWKCDRHQLKLLAKCPQCEARFKIPALWENGRCDRCQITFGELAAYQKSA

SEQ ID NO: 34 - protein, 713 aa, unidentified
  REGION 1..713: note = Description of Unknown: MG64 effector sequence
  REGION 1..713: note = MG64-7 effector
MSQITIQCRLVASASTRQKLWKLMAEFNTPLINELLILAYQHPDFKTWQHKGAIPAGIIKQLCEPLKTDARFVGQPGRFFASAIATVSYIYKSWVKVQKRLQLQIDGKTRWLEMLNSDTELVEMAGVALDTLRATATELLNQLNPQPTTEESPKKKGKKAKKTQQPQGERSLSKILFDTYRDAEDIQTRCAISYLLKNGCKIRSQEEDSKKFAQRRRKVEIQIQRLTDQLASRVPKGRDLTATKWLESLLEATRKVPKNEAEAKSWQDSLNRQSSTLPFPVAFESSEDMIWFRKLKLNNIPIKLWTILLYIDYLIVILFVRDSLQNEALWFKNFKINNIHVLTKLWMILLNINSFAGVLYLGGVLKKYQQRIYVHFNGLSDCTFEIYCDSRHIHWFKRFLEDQQIKRSSKNQHSSSLFTIRSGRIAWKSAQGKGKPWNVNRLMLYCCVDTRLWTAEGTKLVVEEKAEEIAKTITRTKEKETKEKVQLNDKQLAYIKRKNATLTRINNPFPRPSKPLYNGQSHILVSVSLGLEKPATLAVLNAMTGKIIAYRSVKQLLGKNYKLLNQQRHQKQALSHQRKIAQTLAAPNQFGDSELGEHIDRLLAKEIIAVAQKFNAGSIVVPNLDNMREQVNSEIQAKAEEKCPESIEAQKKYASSYRRSVNQWSYRRLIDCITNQAAKAGIVIEENKQPIRASPQDKAKQLALSAYHARKKS

SEQ ID NO: 35 - protein, 587 aa, unidentified
  REGION 1..587: note = Description of Unknown: MG64 transposition protein sequence
  REGION 1..587: note = MG64-7-B transposition protein
MKDAESATNSPMTHASIADAENGKAEANIIVSELSDEALLKMEVIQSLLKNSDRSTYGELLKQSAEKLGKSVRTVRRLVDKWEKEGLAGLVQNQRDDKGKHRVDKYWQEFVLTTYKENNKGSKRMTRQQVFIRAKARADELEIEPPSRMTVYRILKPIIDKQEQAKSIRSPGWRGSRLSVKTRDGKDLQVEHSNQVWQCDHTRVDVLLVDQHGKILSRPWLTTVIDSYSRCIMGINLGYDTPSSTVVALALRHAILPKQYSLEYGLHEEWGTSGLPQNFYTDGGKDFRSNHLQQIGVQLGFVCHLRDRPSEGGSVERPFKTLNTELFSTLAGYTGSNVQERPEEAEKEASFTLRQLEKMLVRYIVDNYNQRIDARMGDQTRFQRWESGLIAMPDLLSERDLDICLMKQTRRQVQRGGYLQFENLMYRGELLAGYAGESVVLRYDPKDITTILVYRIEEGKEIFLARAYAQDLETEELSLDEAKASSRKVREAGKAISNRSILAEIRERETFPTQKKTRKERQKLEQAEVKKAKQLTPAETEEEIIVVSIDAKPTAKNPLESELCTESGEPDMPEVLDYEQMREDYGW

SEQ ID NO: 36 - protein, 278 aa, unidentified
  REGION 1..278: note = Description of Unknown: MG64 transposition protein sequence
  REGION 1..278: note = MG64-7-C transposition protein
MVAKEAQEVAKQLGDIPVNDEKLQAEIHRLNRKGFVPLEQVQTLHDWLEGKRQSRQSGRVVGESRTGKTMGCDAYRLRNKPKQEAGKPPTVPIAYIQIPQECGAKEFFGVILEHLKYQVTKGTVAEVRDRALRVLKGCGVEMLIIDEADRFKPKTFAEVRDIFDKLEIPVILVGTDRLDAVIKRDEQVYNRFRSCHRFGKLSGEEFKRTVDIWEKKVLQLPVTSNLSSKTMLKTLGEATGGYIGLMDMILRESAIRALKKGLQKIDLNTLKEVTAEYR

SEQ ID NO: 37 - protein, 167 aa, unidentified
  REGION 1..167: note = Description of Unknown: MG64 transposition protein sequence
  REGION 1..167: note = MG64-7-Q transposition protein
MESEHIKAWLFQVEPFEGESLSHFLGRFRRANDLTPGGLGSQAGLGGAIARWEKFRFNPPPSHQQLEKLAVVAGIDAGRLVQMLAPAGVSIKLEPIRLCAACYAESPCHKIEWQFKETRGCKHHKLRLLSECPNCGASFKIPALWVDGWCHRCFTLFGEMVNHQKPC

SEQ ID NO: 38 - protein, 739 aa, unidentified
  REGION 1..739: note = Description of Unknown: MG108 effector sequence
  REGION 1..739: note = MG108-1 effector
MSFQTVRCVLCASEEMRQKVWLWTEKYTLLVNELLDQVASHEKFSEWQAQGSIARKEVEILLKSLKASDQFKGLPGRFYTSAVLMVQYTYLAWLALQKKRHFKIIGKQRWLEVLEADLEMASNTDFEWQEIQSRAFELLAQTKKSSDTVSAKPVKKGAVNQEAAKIQKQTPFNALLERFSSEKDLLNRRAIAHLLRNNLQVEENEESLDKLLFRFEKKRIEIERIEKQLESRRPKGRDPVGTRFMESLEEATALPEHTKSEEIEAELNGWKHQKQVRSLNNLPYPVLFVSITDLIWSLQSNENEQESVKGNQVQPARRGKCKKKQKRSSDRICVNFNGFKKFLGETQGVFKIQCDRRQLPIFRQIVSDWRAYQQLPDDEKFGLGLMLLKSAQLIWKKDQQKLYKKKAKNNSTQHETASGGQLVHPWQTHRLYLHCCINPDLLTAEGTEVVRQKKLPATAKKLEGSKKRQAEVAERIQTSDLGEDSTMLKKLHTNLVNRISANATSLNRLENATPQRPSKPAYHGQPHIIVGVSFSREQLVGVAVVDTRTGRVIEYQSTRLLLSNPAVKAKRGGRTVTQLRLEKYRLVNRRQRQQQQNAIRRSKEQKQNRYARSKSESNLGQYLDRLIASKVIKLAIKWQASSIVVPNLGDIRESVEASLKAEAERKFPNEFERQKKYAKHIRTSFHTWSYGRLTECIRSCAAKAQIPIEQGHQPAQGDLKEKAMQVALHAYYTRQSPKG

SEQ ID NO: 39 - protein, 302 aa, unidentified
  REGION 1..302: note = Description of Unknown: MG108 transposition protein sequence
  REGION 1..302: note = MG108-1-C transposition protein
MSQLQFAEPDRPHSSNEETLFLEEPRRSIEAQAEVERIGKADTYLVLDRDIALFDWLDDQRDTKLCGYIIGSQGSGLPKACQFYRMKHVKRRGRLLQVPASVIYAEVKQHGGPTDLYHAILEEIGHPLAKAGALRDLRSRAWGTLKDYGVKLLIVGNADYLKLESFNELIDLFGKLRIAIALVGTDTLAETLDRRGSAYRRIHDAFLESFDFLNLTPEDIREVIEHWENQFLPTHSRLNFARIPDVRKFLELKSKGRIEPLYDLLRKIAILKIDEPKLELNSTNLADRLGKRNAPKDRISTR

SEQ ID NO: 40 - protein, 650 aa, unidentified
  REGION 1..650: note = Description of Unknown: MG108 transposition protein sequence
  REGION 1..650: note = MG108-1-B transposition protein
MFRTSADSNENSASSPISELSSPGLVEAGSFKLLQDISPEVQQEMDLIDAIVQASNKCDRKAAIDRACAVLNRHPRSIMRKVQRLRQEGITSLAKGRKDNGQFRVSEQWVNFITKMYARGQKDSLRFSPHKVFVRLTVFAAQGNRLRESKYSDLFKDYPEVLKDLIQGKHPSHPTVYKIIDNYLLQKNTKARHPGAFPGKLVLQTTEGLISLTHSNQVWQIDHTKLDILLVIKVEEDDKVEHEGKKITARPYLTLVVDSYSSCVAGYYLGFEPAGSHEVSLALRNAILPKQYGQEYKLQAKWNIYGVPDYVMTDRAKEFKSEHLKQISFQLGFQRRLRAFPSAGGLVESHFDKINKEYLSEKPGYTGSSVEERPKEAENAACLTLDDLERELVRYFVDHNNQHFYSEDKLTVSERMSKPKRFERWQDGLLVEPEILNERELDICLMKSIPRKVEKYGCVRFETLRYQGECLANKAFIGRDITLRYDQRNIATLLVYSYSTTSQIQEFIGTVQAIGLERERFSLGEWEALKQKMRDRDKEIDNSILMAERMGLIESTKEQIKSSRSKRQRIKKAHTEQERKANRSKVVELYPERAIEDTTVIQENVESTPNSSIQKRVESTQEQQIQQAAPKTRRVRTVAPDWNQIKQDNW

SEQ ID NO: 41 - protein, 200 aa, unidentified
  REGION 1..200: note = Description of Unknown: MG110 effector sequence
  REGION 1..200: note = MG110-1-6 effector
MKLFSVKITYLPEKCDHALLAGRCIKVLHAFMSRNGQFNIAVAFPRWSENTIGNQLVFVSPDYKFLDMLLEQPYFRMMIENGLFETGTVVDLPVSDSYVKFVRNQSIDKMTPAAKARRLRRAKKRALARGEVFDPIAPRSKDVDFFHSIPMESSESEMSYLLRVQRYEVQQANPVASFEVCSYGLSTNESHQALIPSSVT

SEQ ID NO: 42 - protein, 342 aa, unidentified
  REGION 1..342: note = Description of Unknown: MG110 effector sequence
  REGION 1..342: note = MG110-1-7 effector
MQLPNQLSYKRSINPSKAIFYYRRDEDLYPLPVERIKIRGSKSGFSEAYTAKGIKESATIHSLATGNPHTIDTCYLPPAADSLVCRFSLRVSANSLFPDRCSEIAFKDTVSQFLNSYIAKDGFKELAIRYSKNIAMGTWLWRNKEGNTFDVLVRTSQGNEYKFKNAHMLFWDSVWPDESSELLALLSDELAVALTKPRYVWHCDVWAEVKMPFCSEVFPSQCFVDHDDKQSASKVLLTTDIDGVMTACYNADKVGAAIQMIDDWWDESCDFPLRVNEYAADHENLIARRHPSTERDFYQCLQNLRGYTDKLNKVKSVDDIEPDAHFVASVLVKGGMFQGGKS

SEQ ID NO: 43 - protein, 682 aa, unidentified
  REGION 1..682: note = Description of Unknown: MG110 effector sequence
  REGION 1..682: note = MG110-1-8 effector
MSLQDTLQAVKALKAKERYDALKKSFELYSEHVDVTGSELACLTILVNLVSKDAVELEGLRHAKHVLKSDEFWGKFSKVASQLHTHNLKWPDSRVNLKHHIRALPEIGVLPKFGWSGNSGDYRFGRLLTSTFIWQGLEHSLISVWLGDFVAWRKAAYKLGITKVFWYQIKRDLEDLFQESHFPDVVDSYSPELQFPYKDQYLSVTPVASHETQLAIQHVTGLPMHSLSFPHPSALGVLCGSLGGHVRLIRLSPLGNQNVQRSSLGSLSLKNYLNPYVLTAIPAINLYRRIVDVRTYSSLRLKRRARLNALSTLDGILAEWVAPLVQVKLSSNVDGRIDGLGAEEQEFVESSSVDIEEFSRYLNRKLHGMLELGKYSRKFSYHQRLVGVTQKRLASLLRRLLLAETQSESNNTTFIILKSLRINEANGLNNPYVVGMPSIIGLYGFLHRFECQLREIYADISVDSFALHCGEYSYHASNNLPAPSIPDKEMRIKRSGITPEFKFDGKFSIIVKLHCLTDNAVLDVEQIKAAVPERLWGGSVHPPYLYENTEWAAIAYGSADLERYLIRNLFFGNWITPEEEDGFELRKHIEKLNGKNELSLCLVGYKLLEKVKPRSVVSGIHAFCEPVVDLCCLKQTHKVIKPTKSIEQELFWRYVPVSQNCTTLRVSPVCGETHAASQSAEL

SEQ ID NO: 44 - protein, 383 aa, unidentified
  REGION 1..383: note = Description of Unknown: MG110 transposition protein sequence
  REGION 1..383: note = MG110-1-Q transposition protein
MGFLPNSIHLYPDETVESALLRLCKANHFERYIDLSIEIRSWLEEHHPTIAGAFPVALDAVNVYHAKQSSAKRVQSLQLLEQLVGLPRFSLLDISFKHTNAVDAGHFAEVRYKQITIPKSFVRASSVPVCIACLRESNYVRFDWHISKVTCCEKHKVKLLSNCPACNAPLNYMISEDPSHCVCGFNLLETTNSEKGADDWRRRISFDQQGSLSISEQLALLLFLERYFSHVELGDFIGNYKSLIDKHLQILTSKNILLATEKINRVSFSSLTDNFLGDVAQIGSLPNFIKQHIANVVIELALETPRSTIANLGDSLVSMRDAALITGSTIDDIFRLYESGILVVGKRIRNEGRIESFNPLFRLRDVAAIALSYSRYGYSQSAW

SEQ ID NO: 45 - protein, 334 aa, unidentified
  REGION 1..334: note = Description of Unknown: MG110 transposition protein sequence
  REGION 1..334: note = MG110-1-C transposition protein
MGLTDADKAKLREFKDCFCPYTPVTTVLNDLESLYQSSEIGGEQLSMLLRGDTGTGKSAIIHHFCNIKNGINLESIPVLLSRVPSKLTVEDMTRQLLSDLGVFGSSTHRAKNAQSDAHLTNRLLDALKVKNTKMIIINEFQELIEFKGARDRQAIGNRLKLISEEAAVPIVLVGMPWIDEILNDSQWASRLATRTHTLQYFSLSKRIREYFEFLEAIETYVPCEIETSIIDFEISIALFAASCGEMRQLKAILTEAIKLCLISGEPFSKQALSHSFANLYAGIENPFDMPKEKIKVREVEMHSRYIRGDSTHRAAIEPRRLSDFMSLTQILSKK

SEQ ID NO: 46 - protein, 601 aa, unidentified
  REGION 1..601: note = Description of Unknown: MG110 transposition protein sequence
  REGION 1..601: note = MG110-1-B transposition protein
MFDDEFDHDPLKEDVESSNRQSDEASYLSPDLDSYPIKSSEEALVRYELVQFIRERTSKGWTQRNLNPLISEYFSTHRSLERPSWRTVVRWHKKLLEQGDTPVSLIERHHNKGNRNRKLLLDHERFFEAAIDSFLKAKRPSVATAYRYYKDQCLLQGNNINPMSQRAFYDRIDKLNSYEVAVKRFGKYKADIMYGHKGSTLKPERVMQRVEIDHTVLDITLLDDDTGMPIGKPSLTLLKCALSGCIVGYNLTFKAPCFASVAKAICHTLLPKTSSLDIWGIDWPCYGKIEVLVVDNGVEFWIPSMEKMCFELGINVQYNPVKKPWLKPFIERNFKTINSLLLDELSGKTFGNIYVRGDYDSVKNAEIPFGVFVYVFEKWAAEVYNCSPDSQGMKVPSIIWQDGIEKFPPARLSDSDIRELPKITGFKESRKIQSSGITYKHLRYDSTDLAEYRKQYWSNTRKELVTIKIDVDDLSKIFVYLPEIEKYLTVPCVDQEYTKNLSLDQHLITRTYTKKRNKLLGKSELELAKSRDEIRDAVEQHDKKAATSKKVTTSKKVAQFKGYSNESVKNKVAKPISSDDKSRAEHAEMSELEALWNSFRK

SEQ ID NO: 47 - protein, 207 aa, unidentified
  REGION 1..207: note = Description of Unknown: MG110 transposition protein sequence
  REGION 1..207: note = MG110-1-A transposition protein
MYNRNLRKPSPNKNLYKFVSRKNHSTVMCESGLEFDACFHLEFSPSIASFESQPTGIEYQADNKIRRYTPDFKIVKNTGEIEYIEVKPERIHSTKKFRDEFEYKRAAYRALGFKLILVSEKQIRSEKLLSNLKILHRYASTNFSELHKLALTHIKKFKSLSIRELAIKLGIRICDCIAACALLIGIGAVKADLESDFLCEHSLLNEA

SEQ ID NO: 48 - protein, 215 aa, unidentified
  REGION 1..215: note = Description of Unknown: MG110 effector sequence
  REGION 1..215: note = MG110-2-6 effector
MTDKRYFFAIHYVPAKADCGLLAGRCISTLHGYLLNHAVTQIGVAFPCWSNKSIGRTIAFVSEHSSHLTQFRERTYFQTMQGDGLFELSPVLEVPDDCAEVRFIRNQNLAKLFVGERRRRLLRSKRRVAERGELFVPKAPAINQEIAPFHCALIQSASNAQSYVLHIQKQSCDSEDSTNTYSRYGLASPNSYRGSVPELSSSISTLFPDSLYLKR

SEQ ID NO: 49 - protein, 345 aa, unidentified
  REGION 1..345: note = Description of Unknown: MG110 effector sequence
  REGION 1..345: note = MG110-2-7 effector
MELCKHLSYSRSLSPGKAVFFYKTADSDFVPLRVETARVVGQKSGYSEGFDANFQPKNVERYELAYGNPQTIEACYVPPNIVELYCRFSLRIEANSLSPDLCSDPGVVKTLKQLAGLYQEHGGYIELARRYSRNLLMGTWLWRNRHNQGTRIEVKTSQGSHFAIDDVRHLDWFSEWPALAEAQLNQLAAEMAIALSRRDMFWFADITAVMKTGFCQEIFPSQRFTERPDNHDEPSKKLATVECLDGQLAACLHAQKVGAALQQIDDWWAEDADQPLRVHQYGADSKNLTSMRHPVSGLDFYHLLGRADEFLELMRSSPASSPLPGEIHYLMAVLTKGGLFQKGKG

SEQ ID NO: 50 - protein, 710 aa, unidentified
  REGION 1..710: note = Description of Unknown: MG110 effector sequence
  REGION 1..710: note = MG110-2-8 effector
MPLHELMNLHDQAEQDKALRRAFAPYTPDIELEGEEYRALVVLVNLTLKRDQAVDLCDESQAQRLVNDDKHLEHCVHPVAWLHSHNLKYPDTRVSGQRLVIDAPPLIPGVVTSAGLQNRLGWANNSADINYAKLFCSSFLYQGLSSNLALQLVADVPAWTGAFRQLGVANTAIAALQAQLAHHLAATAIPSEVSVYSKQVRFWYQGDYCAITPVVSHALMAHLQHMIYEKRCSHLTISHDHPSSVGNLVGAVGGKISVLNYPPPISIPKRRHFSQSRTQRLNEGRSLFDRGMLYDRIFPQALEHLITPAGLTRRQRKQSRLSAMRYLRRQLVAWIGPVIEWRDEIQLLPNIELPITPERLEWKIVTSPVAELPDLATELAGVFHLELQTHHATRRFAYHPELLIPIKSQLRWLLNKLADDENRPSPQHAATSCYLHLSGLRVYDALALANPYLCGIPSLSALAGFCHDYERRLTALLKRPVRFTGVSWYLSHYHLLSGKSLQEPLSPMHAREVSAIRRPGLIDSKYCDLGMDLVIALQVDDEHPLPSANEQDLLQAAFPSRFAGGCLHPPSLYEGKPWCSLYTNRNELFNTLSRLPRTGCWVYPYQSRISNLDDLIDTLSMDRRVRPVSTGYVFLDEPRVRAGSVEKCHVYAESALGLALCVNPVEMRLYGNNHFFNHGVWQLTAINRAILMTGAGNMEQRHGAMQTSEL

SEQ ID NO: 51 - protein, 409 aa, unidentified
  REGION 1..409: note = Description of Unknown: MG110 transposition protein sequence
  REGION 1..409: note = MG110-2-Q transposition protein
MRLLIRPEPYPDESLESYLLRLSQENGFERYTLLSGAIRDSLLVQDHEASGAFPLELARVNVFHAQRSSGLRVRALQLFAQLTDLDPACLLQLSLMHSGLHFGSGHACVHRAGVDTPLGFIRTGPIPVCPACLRESAYIRQHWHYSPYLACHQHGSQLLHLCPSCGEALDYQHSESFTHCRCGFDLRTAITQPASAEAQQLSTLICGARFESTNPLLSSEHPSLMFGALYWYWLRNKRADAGQPEFGTLTMAIDYFSAWPANFHHELQQMAQKALQTQTRLLNHTAFREVFGSVLTDCRQLPTRDSHRNFILRGLLDYLTELVCANPKTRVANLADIQLSAIEAAALLGTSVEQVFRLLQDGYLTPAHRRIHGGLKPHEPLFHLRHVIECRQAHRSTFNDAYHPYLPAW

SEQ ID NO: 52 - protein, 335 aa, unidentified
  REGION 1..335: note = Description of Unknown: MG110 transposition protein sequence
  REGION 1..335: note = MG110-2-C transposition protein
MELSSTDADKLKSFIDCYVETPLLRIIQDDFDRLRYDKQFAGEPICMLLTGDAGTGKSSLLRHYMAQSPEQSGQGFVRKPLLVSRIPSKPTLESTMVELLKDLGQWGSEYRLHRSSAESLTEALIKCLKRCETELIIIDEFQELIENKTREKRNQIANRLKYISETAKIPIVLVGMPWAAKIAEEPQWASRLMVRRTIPFFKLSEDAESFVRFVMGLARRMPFATPPKLEAKHTIFALFASCHGSIRRLKHLLDESVKQALAAHSETLQHEHIAVAFALFYPDQINPFLQPIDEIQTCEVKQYSRYEIDAAGKDEVLIPTQFTDKIPISQLLKKR

SEQ ID NO: 53 - protein, 630 aa, unidentified
  REGION 1..630: note = Description of Unknown: MG110 transposition protein sequence
  REGION 1..630: note = MG110-2-B transposition protein
MMCAQQSSEHPSGLFEDEFALPEPAASSTPSESTVTGISATFDSFPDELKNEALHRLKYLQWIEARLIGGWTEKNISPLLVEAASILPPPVPNWRTLARWRKNYIQQGKKIIALIPRHQAKGNSQSRLPLSDEIFFEEAVHKYLVDEEPSIASSYQLYKSKVELENKTIVQNPIKILSYKAFYDRIKMLPAYQVMKCRKGLHLANAQFKAIGSHKRPTRIMERVEIDHTPLDLILLDDELLVPLGRPCLTLLIDCYSHCVVGFNLNFNQPGYESVRNALLNSIPQKNYIKDKYPVIEHEWPCYGKPETLVVDNGVEFWSNSLEQACLELGINIQYNPVRKPWLKPMIERMFRTINCKLIDPIPGKTFSNYLEKGEYNPEKDAVMRFSVFLEIFHQWIIDIYHYEPDSRHRYIPILSWQYGFDRLPPAKVTGEDMAKLEVILSLCIRCKHTRGGVEHLYLRYDSEEFASYRMKYPSKTDGKQYVLVKLNPRDISYVYVFIDKIGEYIRVPCVDSEGYTRGLSLQAHKINVKLHRDFIGKKIDVVSLAISRAKIESRIIKELTEVRQTLKKHNIKGINKIAKYRDIGSQTAANLLSSTQTPENTNDNPVQPKTDQLPLEDDWDSFTSELEPY

SEQ ID NO: 54 - protein, 207 aa, unidentified
  REGION 1..207: note = Description of Unknown: MG110 transposition protein sequence
  REGION 1..207: note = MG110-2-A transposition protein
MYRRHLKHSRVKNLFKFVSAKMNTVFTVESSLEFDTCFHLEYSPAVKTFEAQPEGYYYIFEDRECPYTPDFRVVDERGNPYFIEVKPSAKVATPDFLQRFPIKQQKAIELRSPLKLVTERQIRVDPILGNLKLLHRYSGFQSFTPLHIQLLGLVRQLGRVSLAKLSGSTGAPPEEILATTLSLMARGLIHSDLTEREMGLSSVVWAI

SEQ ID NO: 55 - protein, 400 aa, unidentified
  REGION 1..400: note = Description of Unknown: MG64 effector sequence
  REGION 1..400: note = MG64-8 effector
QFNIENETGSAPNAKKRKKSKSANDPNLMSYLFKAHKATEDILSHCSISHLLKNDCKVSETEEDPDKFAHRIHRKQKQIEQLDAKLNARLPKGRDLTGEEFFRTLETATHQISDNVIQAREWDAKLLTRPATLPYPIIYGSSTDLRWGKTKTGRITVNFNGIDKYLNAADPNLKEWFKTHEEHPFQLYCDRRQLHFFQRFLEDWQAYQANKDTYPAGLLTLSSAILIWTECEGKGDPWNVNHLSLHCTYDTRLMTAEGTLVVQQEKSAKAAKNLEREKSDPRNQSTLNRLNNLPARPSKQPYQGNPEISVGLSIGLTNPITAAVVNVTTGEVLTYRTPKTLLGEQYRLLNRHRDRQQQNTLQRQKNQKRCVRYQPSESELGEYVDRLLANKIIQLAQQYQ

SEQ ID NO: 56 - protein, 748 aa, unidentified
  REGION 1..748: note = Description of Unknown: MG64 effector sequence
  REGION 1..748: note = MG64-9 effector
MGMKTIQSDLIVRLDPNPRRGKKNLDDLSEFDAVAWCRLCERAYQHTLLIDQISEQIKLNPDVIAWIYAVQNSEDQNSADMDTDTDSEQGYSWLPDGLINGLCALLKNTPQFEGMSGRAYTSATDRVEENFKGWFANHQKLIRQIKGKQHWLTVLESDAELAENSNFNQSEIENQAKQILNEIEADNESKDESVNDNRRVFSILFDRFEATEDVLNRRAIIHLLKNGGKVRWEPKKSRNRKSQKHPSKPMTFEERLVAKRIEIARLEKQLLSQLPRARNLFPDIAFEESLAEVVALPKLSDEEQVEAEQVESEFYNWHESITFKLLEFLREPKSLPYPISFGYEDVRSWQINQAGKIFFKLNGWGDLIFEVRCHRRQLPLIKSFLKDWQTKEQCQEGDQYSGSLMLLRSIELVWKPKPVNEQNDIQLCSQCEVFQQYPSKGFWSECKLSIHWSYDSDALSKQGLEKVRQRKLEPQLEKLRKKQEELEEKQQLLQSIEEVPEALRSKAQSKKMRSLTKAIQELQDDLAKPRPKLDCLQNSLLFDRPDRPLHEGVPNIFVGVLLDLDKHLVVTVVDAMRRKILAIRNARSISKEGYDLLQSYFRQRREHSKERQIDQKAHRHVHQTESNLGQHVARLFAKGIVELGQKYKASTIVIPETDGWRDRLYSQLVASAAIKCKGVKKAMTLYTKQHGEKLHQWDYNRLSQAISDRAATDGLKVMRQNTVYEEDAFQQAANLAIAAYDSLNSVEK

SEQ ID NO: 57 - protein, 422 aa, unidentified
  REGION 1..422: note = Description of Unknown: MG64 effector sequence
  REGION 1..422: note = MG64-10 effector
SWQLNQEGKICFKLNGLGDYLFEVRCDRRQLGIVKYFLQDWQTQNKNKNEYSGGLTLLRSAELLVKPKLGKQNAKLPPIHDRQAVVTAYKLSLHCTYDTDYLTHQGLECVRQRKIANQLKGLTDKKAKLTKQQEQLQQLEQEMQQEQIGTSAKRSKRHAQRLKQIEQLKQSISKLQAAIQAELERPRPKLERLQQSQLFQRADRPLYAGVAHLFVGVCLDLDQHLVVTIVDAMRHKVLTKRTVKQIMGEHYPLLQRYRRLKQQHPKQRRQDQKVGRHNHLSETGLGEQVACAIANGLLSLAQQYKVSTIVLPETKGWRERLYSQLVARAKIKCNGSKKAMARYTKAYGKRLHQWDYNRLSRAIETEAQTVGVTVIFQRLEFQANAEQDNQPADEADEQDNQRVNPFELALQIAIAAYDSLQA

SEQ ID NO: 58 - protein, 404 aa, unidentified
  REGION 1..404: note = Description of Unknown: MG64 effector sequence
  REGION 1..404: note = MG64-11 effector
MAESTLWEKSEPEPLAQLPTLPHTLPYQIRFESSDDLYWSLLPKGKQEDAEKNEGKFEQAEPLPRPAPKRKRRKRRKIHYQASICVRFKGLAEHSFRVQCSHRQLPIFQQCLDEWQLLNALSDDVKFSLAVFPLRSARLVWCKDKQASKHSKNPKAEWNQYRLYLHCTIDRRTLTAEGTEELRQQKLVEAQKQKEASVKKQKPPKIPAPLTEEQRQAKNQAEALKRNHSTLLRLQNPSPPRLSKPCYVGQPHLVVGVCFSRRDRASAIVYDIQQHQEIEYVGVRELLTDESAEEHYRHFSSNPQRKGKRSLEQMRLEQYRLVERFYREQEKYQQRRAEAQECGLELEDESVSNLGEYLDRLLAARIVEFAVKQQASSIVIPELGNIRESIECEVQMQMENRGFE

SEQ ID NO: 59 - protein, 731 aa, unidentified
  REGION 1..731: note = Description of Unknown: MG64 effector sequence
  REGION 1..731: note = MG64-12 effector
MASGILRTIQARLVPSEWMPLDQKASEDSGESQPVPIAVVSSFVEDCTLQLGLTSTRLCRALNECINQHSDFEVWKEGGSIPPKALKEIWNSIRTMPPYQDLPERFLRSAWLRVEGIYASWFAIRTRLAAKQDGLNRWLSIVRSDEYLTKTCDCSLEEMQARATQLLTEPSGSSLSALFDAYFALVKADGSLLDRCAIVHLIKNGCAVASKPEDLKKFAATCQKKRQQVERITKQLAAPAPQVRDLGEAAEAALTDSVERIPSNNADFVAQLAALQRKPIPLPYAVVFYSNDDLEWHQIKRRNSATEKIEERIFVKFKGLNKYLRNRVKYNLEAQLAQILESSGLKLDDISWKIIRRKTAQSTVKYISFKLKTPDPTLKQHLKEQFEQIDLEDKYNLQKEYGFELCCGYRQLADFQTFLSDWQLYCTNRDRYSVSPFALRSAALDWQKEVKQGKSKLQVYLRCTLDQQELTAEGAERARAKAATELNKKIAKLEAAQEQGEVLSEEQQRDLERARSQLRGAEHPYPRPSQPLYEGNPDILVGVCLSWEQVATVAVVNWSTRQVLVYRTIHQLMGEDYPLLSSYRHEQPRNANKRHKRQKRGQSSNISEASKGKHIDRVIAKRLVEVVVEFNAGCLVLPNLNGLRENLQAELMAKAAWKHPGDVAKQKAYLKQYKKNLHCWPYRRLAQFVKERADKIGVPIELGQQPREGDFKEQAVQVAWSFHEAQQADEG

SEQ ID NO: 60 - protein, 741 aa, unidentified
  REGION 1..741: note = Description of Unknown: MG64 effector sequence
  REGION 1..741: note = MG64-13 effector
MAIKTLRSQLCATEETREYLWHLFITYTLLINQLLERLPKDAKFSDWRKEGRVPRKAVAALCNKVLEEDGSLKGLPARFYTSAVLCTSYTFASIFAIQNKLRARCEGKQRWLNVAEHDLELAETTAFSLEEIRERAAQVLGQAESERQECLDEEEEPSNIPSIMSLLFKLLDKSNDLLSYRAIAHLLRNDCKVSYEEEDPEKLALRLSKKRIQIERLQEQLNSQLPIGPDPTGERTNQFIEEAIFLPRDSDSSENRIKEFETWEDLLPERIANLSTQLETLPYPLLLGSNDDVSWSWETISTAASSSSTSPNPSPKRTRTRRRRRKAKKRICVSFKGKGLSQIRLRLYCDRRQLPIFRQFVEDSKANKARNKKEKFSLALYPLRSATLLWAEDPKLSTEKNRDKPWNSHRLYLHVTVDTRLLTAEGTEQVRQEKIELMHKFLKGLVEAEETQEANEDEEAFLVAQKNRSRATKKNQTTLKLLESNPPPPRPRQSAYRGNAEIAVKVAFSREHVVGVAVSDGQQPILDYRDVETLLIDSHLELLEQRSHKLRNQPERLRKVKLGDSKKAKKAKRTRYKPKISTRQLQLQPYRLLKRWHRLKRKNVTERRAEQKHGLYRQSQAETNLAHQINHILARNIVDLCQKWSAGTIILPEFGDLRESIESEIQAKARRKYPDDNVERQKQYAKEFRMQFHRWNYKHLSECIRLRAAKVGIACIAGQQPRLGTLREKAIAVTAIPPKPK

SEQ ID NO: 61 - protein, 642 aa, unidentified
  REGION 1..642: note = Description of Unknown: MG64 effector sequence
  REGION 1..642: note = MG64-14 effector
MSIITIHCRLIASEPIRRHLWQLMSRSNTPLINDLLKQVSHHADFETWQTRGTVPSNAIRDLCEPLKEVYPGQPARFYASAILMVTYTYESWLALQQTRRRRLHGKQRWLNVVKSDAELLGLSGSTLESIRQRAQDILSQLSTEMETQSAPNPKKRSQRQGQTHSSNDASLMPRLFTAYDTADDILSQCAIVHLIKNGCKISETEEDSEKFAHRIHRKQKEIDQIEAQLQARLPKGRDLTGDVFLETLAIATQQIPETVIQAREWQAKLLSRPASLPYPMIYGSSTDVRWGKTANDRIAVSFNGIDKYLKEADPEIQEWFKLHKEYPFRVYCDQRQLPFFQRFLEDWQAYQANKDTYPAGLLTLSSATLAWREGEGKGEPWEANHLALYCAFDTRLMSAEGTLEIQQEKSTKALKNLTHDNPDPRNQSTLNRLKNVPDRPSRKPYRGNPEILVGLSIGLADPVTAAVVNGRTGEVLTYRSPRTLLAEHYHLLNRYRQQQQQNALQRHRNQKRGVTYQPSESELGQYVDRLLANSIIQLAQTHQAGSIVIPSLTHVRELLASEITAKAERKSRIVEVQDKYAKEYRIAIHRWSYNRLINAVCSKAQQFGMTVESGFQPLQGNPQEQAKDIAIAAYYARTIAIK

SEQ ID NO: 62 - protein, 828 aa, unidentified
  REGION 1..828: note = Description of Unknown: MG64 effector sequence
  REGION 1..828: note = MG64-15 effector
MSFKTIRSLLCATEETREYLWHLFLNHTLLINQLLEKLPQKTAFAKWRKSGRIPRKAIVAACNTSLQEDESLEGLPARFYTSAVLSVSYTFAFIFAIQNGLRAKVEGKQRWLNVAEKDLELAQTTSFSPEAIRAVAAQVVDQAEAERQREFEARENRTGQPLPLMTVLFKFWNKTKKPLRRRAIAHLLRNDCQVNSEAEDPDKLTLRLSKKRIQIERLQKQLDSQLPTGRDPLGDRIEKFIEDAIAFAEHSTFVPTQFWLKWHRLLLDVQSSEPMLDLWFFGWTYYRLNAIAEFEAWEQSLSERTANLSTQLKSLPYPLLFGSTDDLYWSWEKQVQPKTPQPAPAHPQASADPTIKRKPKRCRTRKRKKKSTPRICVSFKSRGLSHLCLRLYCDRRQLPVFRQLVEESEANKARKKPDRFSLALSPLRSAGLMWVEDPQQLHKKKHWKLKNLWLKWFCEMSSQAFPDEIREQWFQSLVYLCLSLPWQTAAIDPRLLTAEGTEVVRQEKITLMGKFLEGSKKADEKQAIEELSKKKQAKLLTAEKNRCKVTKKNQTTLTLLQSNSPLPRPSRVAYQGNPDIAVKVAFSREHIAGVAVSDGCQPVLDYHDIKSLLVDPRVDLLEERSHKLRNQPERLWKATLATQKSKPSKSKYPRSKPKISTRQLQLQPYRLLKRWRRLKRENGAKRQLEQRHGLYRPSQAESNLAQYINHLLARNIVDLCQRWSAGSIILPKFGDLRESIESEIQAKAKRKYPDDNVERQKQYAKEFRMEFHRWNYQHLTQCIRSRAATVGVKCVAGQQPRLGTLREKATLAVSAEQRVVTAIPPKPK

SEQ ID NO: 63 - protein, 601 aa, unidentified
  REGION 1..601: note = Description of Unknown: MG64 effector sequence
  REGION 1..601: note = MG64-16 effector
MTKLTIHCRLVAEEETRRQVWEMMAGRYAPLIATTLEQVSQHKDFPQWVATGELPTQVVKNLVNQAQSGLPARWCASAQRQVQETYKAWLTKRRKLQQKLQGQQTWLSVLRPDAELAKNAGLSLEEMKIRAQALLHREINNWFQVYQQCQDVVERSIFAYLLKHRLTVPAEPENTDKLRQKRRQVEIKIERLETQLAGRSPQGRDLTGSRYATALNDGEQCDWEDDADFLAWQAEILSRPDSLPPPVEYATNTDMTWHKDEQGRLAVTFNGLGKLKFKIACDQRQLHWFQRFYQDQEQFKAQKGQRSQALFTLRSAELLWKPGNRSGDPWQANFLYLHCTVDSRLWTQEGTAMVQQEKAKKSQAIVKKLSERSDLTAQQKDCLQRHQSTLARLHMGYDRPQRRMYQGKSHLVVGISLDMENLVTVALVDVVKQKVITGCTMKSLLGQDYALVQRLRYEKRQNSHLRKVAQERGSKIVNYEANLAIHVERLLVKAIIHFAQQHLAGSLCVPTLKDIRETIQAHLQCRAEERFPDSKELQRRYAKEYRINAHRWSYNRLLKLLNQQAKFAGLVVEQGVQSAGETALERALGVALSAYYQRSAA

SEQ ID NO: 64 - protein, 605 aa, unidentified
  REGION 1..605: note = Description of Unknown: MG64 effector sequence
  REGION 1..605: note = MG64-17 effector
MTKLTIQCRLVAEEETRRQVWEMMAGRYAPLIATTLEQVSRHKDFPQWVSAGEIPAQVVKSLLHSAKTSYGGLPARWYASAQRQVQEIYKGWLKRRRQLQHKLQGQQTWLSVLQPDAELAQSAGLSLPELQQRAQALLAGDTVTNWYHAYQQCQDAVERSIFAYLLKHRLTVPTEPEDTDKLKRKRRRVEIRIERLETQLAGRSPQGRDLTGIRYATALHEGEQCYWEDDADFLAWQAEILSCPDSVPPPVEYATNTDMTWHKDEQSRLWVTFNGLGKHKFKIACDQRQLHWFERFYKDQELFKAQKGQRSQALFTLRSCELLWKPGNRSGELWQVNCLYLHCTVDPRLWTQEGTAIVQQEKAQKSQAIVKELSERSDLTPGQEDYLRRHQSTLARLHTGYSRPQRRMYQGKSHLSVGISLDMDNLVTVALVDVVEQRIITGTTMKNLLGSGYALVQRLRYEKRRNAHLRKVAQQRGSKIVNHEANLATHIERLLAKAIINFAQQHLAGSLCVPTLKDIRETIQAHIQSRAEERHPNSKELQRRYAKEYRINAHRWSYNRLLELISQQAIVAGLLVEQGLQGSGGTAIKRALGVALSAYYQRSAA

SEQ ID NO: 65 - protein, 613 aa, unidentified
  REGION 1..613: note = Description of Unknown: MG64 effector sequence
  REGION 1..613: note = MG64-18 effector
MSIKTIRCRLVASESTRKALWELMAGKNTPLMNHVLALVSSDTNFDSWRQKGEIPASIIKNYINTLKHEPQFAGQPWWCYASSEKQATRIFDNWLATQKQLQSKLNGKEQWLSVLQPDSELASQASCTLAALRRLASQINKQGLTFNQLFNLYTTVQVLKQRAAIAYLLKRQGKLEPNEEDPEQIARKRRKTEISIQRLKKQIQARVPKGRDLTGSEYTAALDECIQTAITTDEEYEAWQGKLTAKTVSVPFPLICESSEVLKWSQTSSGRIVVKFSGLHGLSFDIYCDKTHLRWFSRFLADQEVKKASGGKHSAALFTLRSATLLWRPNKHPSKGDPWSTNYLELHCTVDTRLWTAEGTEQVRREKSTEVEKILTEIGSKDSLSNNQLVYIKRKQATLTRLQGNFDRPHQSPYCGDPDISVGISMGLDKPVTLAIVNLKTGQVSAYRTTRQLLGSNYCLLGRRRKEQERASHRAHINRARHRSDYHTYKESQVGTQIDHLLACSIIEVAMQYKASSIVLPDLEYIREVVEAEIKERAERRIPDYSEGQKRYAKEYRKKVHSWSYRRLHNFIKNKCEAVGIAIEVQRQETIGTPQQKARKLVEKAYANRCFVA

SEQ ID NO: 66 - protein, 543 aa, unidentified
  REGION 1..543: note = Description of Unknown: MG64 effector sequence
  REGION 1..543: note = MG64-19 effector
KNLVNQAQSGLPARWCASAQRQVQETYKAWLTKRRKLQQKLQAQQTWLSVLRPDAELAQEAGLSLPELQQRAQALLAGDTITNWYQAYQECQDVVQRSVFAYLLKHRLTVPNEPEDTDKLKRKRRQVEIKIERLETQLAGRSPQGRDLTGSRYATALNDGEQCDWEDDADFLAWQAEILSRPDSLPPPVEYATNTDMTWHKDEQGRLAVTFNGLGKLKFKIACDQRQLHWFQRFYQDQEQFKSQKGQRSQALFTLRSAELLWKPGNRSGDPWQANFLYLHCTVDSRLWTQEGTAMVQQEKAKKSQAIVKKLSERSDLTAQQKDCLQRHQSTLARLHMGYDRPQRRMYQGKSHLVVGISLDMENLVTVALVDVVKQKVITGCTMKSLLGQDYALVQRLRYEKRQNSHLRKVAQERGSKIVNYEANLAIHVERLLVKAIIHFAQQHLAGSLCVPTLKDIRETIQAHLQCRAEERFPDSKELQRRYAKEYRINAHRWSYNRLLKLLNQQAKFAGLVVEQGVQSAGETALERALGVALSAYYQRSAA

SEQ ID NO: 67 - protein, 605 aa, unidentified
  REGION 1..605: note = Description of Unknown: MG64 effector sequence
  REGION 1..605: note = MG64-20 effector
MSKLTIQCRLVACEDTRRQVWEMMAGRYAPLIATTLEQVSQHKDFPQWVSAGEIPAQVVKSLLHSAKTSYGGLPARWYASAQRQVQEIYKGWLKRRRQLQHKLQGQQTWLSVLQPDAELAQSAGLSLPELQQRAQALLAGDTVTNWYHAYQQCQDAVERSIFAYLLKHRLTVPTEPEDTDKLKRKRRRVEIRIERLETQLAGRSPQGRDLTGIRYATALHEGEQCYWEDDADFLAWQAEILSCPDSVPPPVEYATNTDMTWHKDEQSRLWVTFNGLGKHKFKIACDQRQLHWFERFYKDQELFKAQKGQRSQALFTLRSGELLWKPGNRSGELWQVNCLYLHCTVDPRLWTQEGTAIVQQEKAQKSQAIVKELSERSDLTPGQEDYLRRHQSTLARLHTGYSRPQRRMYQGKSHLSVGISLDMDNLVTVALVDVVEQRIITGTTMKNLLGSGYALVQRLRYEKRRNAHLRKVAQQRGSKIVNHEANLATHIERLLAKAIINFAQQHLAGSLCVPTLKDIRETIQAHIQSRAEERHPNSKELQRRYAKEYRINAHRWSYNRLLELISQQAIVAGLLVEQGLQGSGGTAIKRALGVALSAYYQRSAA

SEQ ID NO: 68 - protein, 638 aa, unidentified
  REGION 1..638: note = Description of Unknown: MG64 effector sequence
  REGION 1..638: note = MG64-21 effector
MGQITIQSRLVASEATRQHLWMLMADRNTPLINQLLEQINQHPDFLTWRQKGKIPVGTVKQLCQPLRSDPRFSGQPGRFYTSAIALVDYIYKSWLTLQQRLQHKLNGQQRWLEMLKSDEELAEISGCSLDTLRQRAANLLQADNPIHPEPSTLPKSGKRRKPKQTETSALSPNISQALFFAYRETEDVKIRAAIAYLLKNGCTLPEKPEDPQKFAKRRRKVEIQIQRLTEQLEGRVPKGRDLTNTQWLAALETATTTAPQSEAEAKSWQDSLLRESSNLPFPVAYETNTDLTWFRNQQGRLCVRFNGLGDHIFQIYCDRRQLHWFERFLDDQQVQKDSKDQHSSALFTLRSVRISWQEGKGKGTPWQIHRLALQCSLDTRLWTQEGTEQVRNEKAADIAKILTKMEDKGNLNDKQEAFIKRKQSTLDRINHTFPRPSKPLYQGQSQIIVGVSIGLEKLATAAVVDASTGKILTYRSIRQLLGNNYRLLNRQRQQQHHNTHQRQIAQRQGKRGLLSESELGQYVDRLLADAIVDLAKTYYAGSIVVPKLGDVRERVQSEIQARAEQVCPDLLSGQQNYAKQHRSAIHRWSYGRLIDQIQSNAKRTGIVVEDAQQPIQGTPQEIARALAITAYQDRLKMI

SEQ ID NO: 69 - protein, 502 aa, unidentified
  REGION 1..502: note = Description of Unknown: MG64 effector sequence
  REGION 1..502: note = MG64-22 effector
ANLLQADNPIHPEPSTLPKSSKRRKPKQTETSVLSPNISQSLFVAYRETEDVKTRAAIAYLLKNGCTLPEKPEDPQKFAKRRRKVEIQIQRLTEQLEGRVPKGRDLTNTQWLAALETATTTAPQSEAEAKSWQDSLLRESSNLPFPVAYETNTDLTWFRNQQGRLCVRFNGLGDHIFQIYCDRRQLHWFERFLDDQQVQKDSKDQHSSALFTLRSVRISWQEGKGKGTPWQIHRLALQCSLDTRLWTQEGTEQVRNEKAADIAKILTKMEDKGNLNDKQEAFIKRKQSTLDRINHTFPRPSKPLYQGQSQIIVGVSIGLEKLATAAVVDASTGKILTYRSIRQLLGNNYRLLNRQRQQQHHNTHQRQIAQRQGKRGLLSESELGQYVDRLLADAIVDLAKTYYAGSIVVPKLGDVRERVQSEIQARAEQVCPDLLSGQQNYAKQHRSAIHRWSYGRLIDQTQSNAKRTGIVVEDAQQPIQGTPQEIARALAITAYQDRLKMI

SEQ ID NO: 70 - protein, 541 aa, unidentified
  REGION 1..541: note = Description of Unknown: MG64 effector sequence
  REGION 1..541: note = MG64-23 effector
ADSLRSDPRFKSHPQRLVASAGRVVNSMFESWLAIQKGNFARLKGKQRWLAMLKSDEELVEYSDCDLYQIRNHAAQILESVAASVPSQTSNPANKKSKQQKSDRKKSKQKDNNIPKALHGLYDSTDNVLTQAAIIYLLKNGSKVSDCEEDPEKFTQKYRKTELQVERLQKQIDANMPKGRDLTGQVWEETLEIATLTAPRNGDEAKEWQNTLLRKPKNTPYPIFYRNKEDLRWYEIEINGKARRRGKNHNSPKKQLCVTLSGFGGHALKVSCDRRQLHWFKRFLEDQQTKKNSDGQCSSSLFTLRSAQLLWHEQKGNGNSWDVHHLHLHCTVETPRWTAEGTEKIRQAERQEVAKRIESLNQKEALSPSQQKYLQRLDSQLKGLDYPFDRPSRPLYTPQPDIIAAVSMGLQYPATLIIMNSSTQEVLATRTIRELLGSNYGLLLRRRRQQQKNAHQRHKAQKRSASDQFGESNLGEYLDRLIAKEIVAIAKVHKAQSVVIPKLARIREILNCEVMSRAEQRCPGSVEIQKRYAKEYRTKIH

SEQ ID NO: 71 - protein, 614 aa, unidentified
  REGION 1..614: note = Description of Unknown: MG64 effector sequence
  REGION 1..614: note = MG64-24 effector
MSQKTVRCRLISTEENRKALWLLMAERNTPLINEALRQLPSHSDFPKWRQKGKLPDIAAKCLIDRLKTDARFANQPVWYCISAQKHVTYIFRSWLAIQRRKQWKLEGKRRWLEILQPDTALAEKAGCSIEALREEAGEIIAEAEIVDPFRHLLAKYRETEDVREQCAIAYLLKRKAELEPEEENLAKLVERHRKTEIFIQRLEAQLDASLPKGRDLTGHLQAEALIQSIHSPLLDDSNYNTWKDALTTEPAKFPFPIMYETTESLVLSRDDRGRILLRFSGLSQQTYKIYCDKPHQHWFERFFEDQETKRVGGDQHSAAAFTLRSAQLMWVPSEKHRDEPDPWNRYYLNLSCTVDTRLWTQEGTKVVVQEKAVKTAQKLTSMQEKKSLTQTQRGYIRRLESTLQRLQNPYPRPSRTVYWGQPEILVGVSMSLDKTVTIAVVNALTEQVLTYRSAKQLLGERYRLLQRARKEIVKISHQGHRQRRKGGRRISQESDVGKYVDRLIAKAIDTLALKYRAGSIVLPNLAYIRESIEAEVQQRAIEKVPDFKDGQKQYAKAYRTQIHRWPFSRLQSAIISKAEQSGITIEIATQQISGSFQDKARELGLQAYAHRSAS

SEQ ID NO: 72 - protein, 440 aa, unidentified
  REGION 1..440: note = Description of Unknown: MG64 effector sequence
  REGION 1..440: note = MG64-25 effector
LKNRQRLPKKAEDPEKLAQRRRKVEIKIERLRDQIESCIPKGRDITGEAWLNTLLTAANTVPQSAQEAKTWQDILLTRSKSVPYPVAYETNEDLTWSKNEKNRLCVRFNGMSDYSFQIYCDQRQLKWFERFFQDQETKKQSQNQHPSALFTLRSARIFWQEHQGKGQPWQKHRLILYCSLDTRFWTAEGTEQIRQEKAAKITKTLSNLQAKDELTDSQQAFLRRQTSTLERINTPFPRPNQAAYQGQPHILVGVALGLEKPATVAVVDINTGRAIAYRSIKQLLGSNYKLLNRQRRQKQRNAHQRHKAQKQNKPNQFGDSNLGEYIDRLLAKAILSVAQQYQASSIVLPKLGDLRELVQSEVKVKAEAKIPGYLEGQEQYAKQYRVSVHQWSYGRLIDNIHQQASKIGIAIEQGQQPTRASPQNQARELAITAYQSRTQK

SEQ ID NO: 73 - protein, 561 aa, unidentified
  REGION 1..561: note = Description of Unknown: MG64 effector sequence
  REGION 1..561: note = MG64-26 effector
QHKDFPQWVSAGEIPAQVVKNLVNQAQSGLPARWCASAQRQVQETYKAWITKRRKLQQKLQGQQTWLSVLRPDAELAKNAGLSLEEMKIRAQALLHREIHNWFQVYQQCQDVVERSIFAYLLKHRLTVPAEPENTDKLRQKRRQVEIKIEWLETQLAGRSPQGRDLTGSRYVAALNEGEQCYWENDADFLAWQAEILSRPDSLPPPVEYATNTDMTWHKDEQGRLAVTFNGLGKLKFKIACDQRQLHWFQRFYQDQEQFKSQKGQRSQALFTLRSAELLWKPGNRSGDPWQANFLYLHCTVDPRLWTQEGTAMVQQEKAKKSQAIVKKLSERSDLTAQQKDCLQRHQSTLARLHMGYDRPQRRMYQGKSHLVVGISLDMENLVTVALVDVVKQEVITGCTMKSLLGQDYALVRRLRYEKRQNSHLRKVAQERGSKIVNHEANLAIHVERLLVKAIIHFAQQHLAGSLCVPTLKDIRETIQAHLQCRAEERFPDSKELQRRYAKEYRINAHRWSYNRLLKLLNQQAKFAGLVVEQGVQSAGETALERALGVALSAYYQRSAA

SEQ ID NO: 74 - protein, 583 aa, unidentified
  REGION 1..583: note = Description of Unknown: MG64 effector sequence
  REGION 1..583: note = MG64-27 effector
QVWEMMAGRYASLIATTLEQVSQHKDFPQWVSAGEIPAQVVKNLVNQAQSGLPARWCASAQRQVQETYKAWLTKRRKLQQKLQGQQTWLSVLRPDAELAKNAGLSLEEMKIRAQALLHREINNWFQVYQQCQDVVERSIFAYLLKHRLTVPTEPEDTDKLRRKRRQVEIKIERLETQLAGRSPQGRDLTGSRYVAALNEGEQCYWENDADFLAWQAEILSCPDSLPPPVEYATNTDMTWHKDEQGRLAVTFNGLGKLKFKIACDQRQLHWFQRFYQDQEQFKSQKGQRSQALFTLRSAELLWKPGNRSGDPWQANFLYLHCTVDPRLWTQEGTAMVQQEKAKKSQAIVKKLSERSDLTAQQRDCLQRHQSTLARLHMGYDRPQRRMYQGKSHLVVGISLDMENLVTVALVDVVKQKVITGCTMKSLLGQDYALVRRLRYEKRQNSHLRKVAQERGSKIVNYEANLAIHVERLLVKAIIHFAQQHLAGSLCVPTLKDIRETIQAHLQCRAEERFPDSKELQRRYAKEYRINAHRWSYNRLLKLLNQQAKFAGLVVEQGVQSAGETALERALGVALSAYYQRSAA

SEQ ID NO: 75 - protein, 417 aa, unidentified
  REGION 1..417: note = Description of Unknown: MG64 effector sequence
  REGION 1..417: note = MG64-28 effector
VEIKIERLETQLAGRSPQGRDLTGSRYATALNDGEQCDWEDDADFLAWQAEILSRPDSLPPPVEYATNTDMTWHKDEQGRLAVTFNGLGKLKFKIACDQRQLHWFQRFYQDQEQFKSQKGQRSQALFTLRSAELLWKPGNRSGDPWQANFLYLHCTVDSRLWTQEGTAMVQQEIAKKSQAIVKKLSERSDLTAQQKDCLQRHQSTLARLHMGYDRPQRRMYQGKSHLVVGISLDMENLVTVALVDVVKQKVITGCTMKSLLGQDYALVQRLRYEKRQNSHLRKVAQERGSKIVNYEANLAIHVERLLVKAIIHFAQQHLAGSLCVPTLKDIRETIQAHLQCRAEERFPDSKELQRRYAKEYRINAHRWSYNRLLKLLNQQAKFAGLVVEQGVQSAGETALERALGVALSAYYQRSAA

SEQ ID NO: 76 - protein, 488 aa, unidentified
  REGION 1..488: note = Description of Unknown: MG64 effector sequence
  REGION 1..488: note = MG64-29 effector
MGKDILDEIASIGVIEDETEWNSIESALLKKQTLMPHPILFESSDDFIWAEPARFQIEDLQAEEEISRDTEAKELKAKNLQAEKESSNTEPRKRVCVCFKSFDEKYTFEVAGDYRHIHAVWQALKERKEYDKNADKNTSALFLVRSATLIWREYKKNDNRIVRRRKAAKKRAKREGLVASTESDSSLAPEFYDPEFPWNRYQLFLHCTIETRYLSKEGTEIDIEKQKKSTAKSIQTLEKNIAELEKKGESAKTRKDRHNRRSGTLRRLESYDNNYERSSKPLYAGQPHIVTGVALGSSGLVTTTISDAISGKILECRGLEALLGKDYELVKRRQFERQLNSRRRTQNQKRGANDQFGESNLGDTIDKRIANAVIDFAKKHQSGFIVLPDMNDYRRRKQSEIAAFAERECGGWKGIEKKFAKAQKEKIHSWSYGRLTTYITNQAEKEGILVRTRRQPIQGSSQEQGKLMAIEAYKDKPKPKKSRPKKSA

SEQ ID NO: 77 - protein, 467 aa, unidentified
  REGION 1..467: note = Description of Unknown: MG64 effector sequence
  REGION 1..467: note = MG64-30 effector
KTAEILAQTTPQPAEARTAKSSKKKQKSKLNGQKQSFDLLFEQYQETDDELTRSAIVYLLKNGRKLPNEPEDPKQFAKRRRKAEIRVERTTNTLHRMQLPTGRDMTDQEWLKTLATAVSNVPEDDGEAASWQAILMSEAHKLPFPILYETNEDLSWFLNDGERLCVTFNGLSEHTFEIHCDRRQLHWFKRFLEDQEIKKASKNQHSCALFTLRSARIAWQEGKEKGEPWNTHRLTLACTVETRAWTAEGTEQIRQEKAAECKKVILGTKAKGNLNQRQEQFIQRREKTIVLMNNSFPRPSRPLYQGQPEIIASVSYGLDRPATLAIVDITTGKAIAYRSIRQLLGDHYKLLTRYRLRQQQNAHRRHNRQRKGASNRIQESQLGEYLDCLIAQAIVSVAQTYQASSIVLPDLGNIREVVEAEVRARAEQRIVGYLEGQQQYAKQYRASIHRWSYGRLSEKIQSQAAQA

SEQ ID NO: 78 - protein, 550 aa, unidentified
  REGION 1..550: note = Description of Unknown: MG64 effector sequence
  REGION 1..550: note = MG64-31 effector
PLINALIRELVSHPEFESWREKGRHPSDVVAKLNRTLKTDAQFAGQPSRFFMSAEKVVNYIFKSWFKIQSRLQQKLSGKQKWLEILKSDEELVELCGQSIDVLQKKAVQVLQEVGKAIDKETSDTIPEKKRANLIRQRLFKKLSSAKQPLPQYAAIYLLKNHCKIPNEPEDVENFAQRRRKTEIQIQILQDQIESRIPKGRDLTGKAWTETLLKAVNSIPQDNQEHKQWQDRLLSMPSTAPFPILFETNEDLVWSANERDRICVRFNGLSEHTFQIYCDQRQLHWFKRFLEDQKTKRASKNQHSSALFTLRSARISWQENDRKGNPWETHYITLSCTVDVRLWSAEGTDEVRHEKAADVAKVLTRLNEKDSLTETQAGYARRLTSTLEKLSSPFERPSLPRYQGKDNIIAGISLGWDKPVTLAIWNTDTQEVLTYRSLKQLLGKDYPLYLRHRREQQKQSHERHKAQKQGKGNRFGTSNLGEHLDRLLAKAIVAIAQQYSAGSIAVPKLDSIRDTLQAEIDAKAEQRIPGYLEGQKRYSRDYKRSIHRWS

SEQ ID NO: 79 - protein, 446 aa, unidentified
  REGION 1..446: note = Description of Unknown: MG64 effector sequence
  REGION 1..446: note = MG64-32 effector
SIFAYLLKHRLTVPTESEDTDELRRKRRQVEIKIERLETQLAGRSPQGRDLTGSRYATALNDGEQCDWEDDADFLAWQAEILSRPDSLPPPVEYATNTDMTWHKDEQGRLAVTFNGLGKLKFKIACDQRQLHWFQRFYQDQEQFKSQKGQRSQALFTLRSAELLWKPGNRSGDPWQANFLYLHCTVDSRLWTQEGTAMVQQEKAKKSQAIVKKLSERSDLTAQQKDCLQRHQSTLARLHMGYDRPQRRMYQGKSHLVVGISLDMENLVTVALVDVVKQKVITGCTMKSLLGQDYALVQRLRYEKRQNSHLRKVAQERGSKIVNYEANLAIHVERLLVKAIIHFAQQHLAGSLCVPTLKDIRETIQAHLQCRAEERFPDSKELQRRYAKEYRINAHRWSYNRLLELISQQAIVAGLLVEQGLQGSGGTAIKRALGVALSAYYQRSAA

SEQ ID NO: 80 - protein, 649 aa, unidentified
  REGION 1..649: note = Description of Unknown: MG64 effector sequence
  REGION 1..649: note = MG64-44 effector
MSQITIQCNLVASEATRQYLWHLMADIYTPFINEMLATIAQHPNFEEWSQNGRIPADVFEDIRKTLKAHPDFQGMPGRWYYAGRDLVKRIFKSWLALRRRLRHQLSGQTHWLEIFQSDDDLVAACGQDLPAIRAEAASILTKIQIEAPNTSKQPKKTKQPKKAGSKTQKPEEEQRNRNLFPALFKEYDGAETELVKCAIACLLKNNCQIPTKAEHPEKFQKRRRKTEIRVERIIEQLARTRLPKGRDLTNEKWLDTLKMAVQQVPKDETEAAAWEADLQTDSSPLPFPIAYESNEDLKWSQNAKGRLCVRFNGLGKHTFEIYCDTRQLHWFKRFLDDQTIKKQGGNSHSAGALTLRSGRISWRLDSSKGNPWDRNRLVLFCSVDTLLWTKEGTEKASQEKASKIAQVISGTKAKGNLTSKQEDFVRKREKTLALLQNPFPRPSRPLYQGSPAILAGVSFGLDKPATLAIVDVTTGKAIAYRSIRQLLGDDHKLLNRQRQRQRQKAQRRRSNQLKFASNRISEGGLGGQIDSLIAKAIVQIAQQYNASSIVLGDLANIREIIESEIQAKAEQKTTLKEIQAKYARDYRASIHRWSYKRLAQKIESNALQAGLIVATIKQPLAGSPQDKARDVAIAGFQSRSVSKILDTGS

SEQ ID NO: 81 - protein, 429 aa, unidentified
  REGION 1..429: note = Description of Unknown: MG64 effector sequence
  REGION 1..429: note = MG64-45 effector
MPPCRPPATRQYLWGLAAEKNTHLINALIQEVVSHEDFEDWRLKGRHPADVVAKACRRLKTEVPFSGQPSRFYASAEKAVNYIFKSWFTLQSRLQRQISGKQAWLDMLKSDDELVELCGRPLEGIQATAAQLLKQIEKTLESEATEESQGKSKEDQVRTHLFKKYHSAKQALNLCAAAYLLKNNSKIPDKPEDPNKFAQRQRKAEIQVQRLQDQIEGRIPKGRDLTGQAWLSTLLTVTTTVPRDNREHKQWQDRLLAQPRTIPFPILFETNEDLVWSRNQADRLCVRFNGLSEHTFQIYCDQRQLPWFQRFLEDQQTKRASKNQHSSALFALRSARISWQENDRKGQPWDAHSLTLSCTIDTRLWSAEGTDEVRQEKAADTAKVLTRLNEKGSLSDTQAGYAKRLTSTLNRLDSPFKRPSQPRYQGKPH

SEQ ID NO: 82 - protein, 477 aa, unidentified
  REGION 1..477: note = Description of Unknown: MG64 effector sequence
  REGION 1..477: note = MG64-46 effector
EQQNRNLFPALFKEYDGAETELVKCAIACLLKNNCQIPTKAEHPEKFQKRRRKTEIRVERIIEQLARTRLPKGRDLTNEKWLDTLKMAVQQVPKDETEAAAWEADLQTDSSPLPFPIAYESNEDLKWSQNAKGRLCVRFNGLGKHTFEIYCDTRQLHWFKRFLDDQTIKKQGGNSHSAGALTLRSGRISWRLDSSKGNPWDRNRLVLFCSVDTLLWTKEGTEKASQEKASKIAQVISGTKAKGNLTSKQEDFVRKREKTLALLQNPFPRPSRPLYQGSPAILAGVSFGLDKPATLAIVDVTTGKAIAYRSIRQLLGDDHKLLNRQRQRQRQKAQRRRSNQLKFASNRISEGGLGGQIDSLIAKAIVQIAQQYNASSIVLGDLANIREIIESEIQAKAEQKTTLKEIQAKYARDYRASIHRWSYKRLAQKIESNALQAGLIVATIKQPLAGSPQDKARDVAIAGFQSRSVSKILDTGS

SEQ ID NO: 83 - protein, 439 aa, unidentified
  REGION 1..439: note = Description of Unknown: MG64 effector sequence
  REGION 1..439: note = MG64-47 effector
LKNGGKISNKEEDPEKFAKRRRKLEIQIERLREQLEARIPKGRDLTDAKWLETLLLATNKAPENEEEAKFWQDSLLKKSSTVPFPVAYETKEDMTWFKNERGRICVKFNGLSEHSFQVYCDTRHLHWFQRFLKDQQIKHNSKNQHSSSLFTLRSGRIVWQEGESKGDPWKVNHLILYCSVDTRLWTAEGTNLVREEKAEEIAKTITQTKVKGELNDKQLAHIKRKNSSLARINNPFPRPSKPLYKGQPHILVGVSLGLEKPTTLAIVDGTTSKVITYRSIKQLLGDNYKLLNRQRQQKHLLSHQRQIAQTLASPNQLGESELGQYVDRLLAKEIVAIAQTYKAGSIVLPKLGDMREQLQSEIQAKAEQKSDLLEVQQKYAKQYRVSVYQWSYGRLIANIRSSAAKAGIVIEESKQPIRGSPKEKAKELAIVAYNSRQTT

SEQ ID NO: 84 - protein, 641 aa, unidentified
  REGION 1..641: note = Description of Unknown: MG64 effector sequence
  REGION 1..641: note = MG64-48 effector
MSVITIQCRLVAKEFSLRYLWELMAQKNTPLVNELLQAIVQHSDFESWLQKGSIPQATIKALCDSLKNDPRFSGQPGCFYTSATSLVSYNYKSWFALQKRRQQKIEGKERWLNLLKSERELEEECDRTLEEIRAKATQILTQFQPQAENAETPPGEATQKKKGKKSKSTPSKGKAASLFNTLFNAYAQTKDPLERCALAYLLKNNGEVSLVEEDLEAYQQRRRKKEIEIERLKEQLKSRHPKGRDLTAEKWLETLEAARTQQPESEAEAATWQANLLRNSSPVPFPVNYETNEDLTWFKNEAGRICVFFSGLREHHFEVWCDNRQLHWFERFFQDQQIKRQSKNQHSSSLFTLRSGRIGWQECTGEGEAWNIHHLKLYCTLDTRLWTAEGTEQVAQEKAAEISNILTKTKQKDDLNEKQLAFIKRGETMLARINTPFPRPSKPLYQGQSEIVVGVSLGLEKPATVAVIDVPGDRVLAYRNVKQLLGDHYPLLNRRRQQQRSLSQKRQKAQKQGAANEFGESELGQYIDRLLAKEIVAIARSYQAGSIALPKLTDIREILHSEMQTRVEKKIPGYKEAQDKYAKQYRVNVHRWSYGRLMDAIASQAAKVGIVIEVGKHPSEGSFQDKAREVAISAYCDRSVN

SEQ ID NO: 85 - protein, 709 aa, unidentified
  REGION 1..709: note = Description of Unknown: MG64 effector sequence
  REGION 1..709: note = MG64-49 effector
MSIITIRCRLVAGIKQQIDKKSIKNFSAEDRALLQKLLDDKSQTDRDNPKDKQKIFLAQSSEDVRQNLWQLFFTSSALIDQLLDRLSQHPNVQTWQQQGKLPDDELKACWLELKTSPLYDEKLPGRFFSSVQSMVKTIYASWLALHQQKQRRLNGLNRLTEIVYSDETLLEMCDSTFAQLQLNAASMLSEIDKEVVNSEKSRSRIDLLFKKYTELPVEDTIGRSAISYLIKHGCKIESKIELTPKFARWFKTKLKQAQRLEHQLAGHFPRGRDLNGIAFLNALETATKDEPQDNPELMLWQSQILRDPSSLPHPIEFNSNTDLRWLKLYRKQYQCKRTVSGDSIDSIELTQRLFVEFKGLTQGSNYVFEVYCDRRQLAIFQQFFDDDRLLRNSSSEEKYSSSLFTLRSAYLLWDRQESQDRYRRHTLSIQTATEPWNTNQLYLHCSIETKSLTAEGMREIQQQKTQKVNNILAKQSKNAYPSIDQQQSQRKNQTSLNLLDRSLPRPSRPIYQGNPQIIVGLIFDPVRPIYLAVVDVTTGKPITYRSTRQLLGDKYQKLSEYRLKQQQNSHHRRKHNQQGQFQQPTESTQGEYLNRLLAKAVIQIAQEFKAASIALPPINHNIEKVQSKIEADAEEDLPEDVGTQKKITRKTSVVIHKWSYHSLLEYVKSNAAKLGITVETVSLPSLGTPSQQAAEVAISAYNSRKHIKK

SEQ ID NO: 86 - protein, 642 aa, unidentified
  REGION 1..642: note = Description of Unknown: MG64 effector sequence
  REGION 1..642: note = MG64-50 effector
MRYAISMSQITIQCQLFADEAARKYLWQLMTARNTPLVNELLRLIALHPDFLKWRSSGKLPTTEVTKLAKTLKTDERFNNQPAKFHISAEKTAIYTFKSWLAIQKRAQQQLEGKISWLRMLRTNEELVADCGQDLAQIRTKAQALILQYQSSEDSAESQKNFRKSLYQAYDIAEPRLGAQNERSLTRSAIAYLLKNRCHIPSDTDEDLKKFLKYRRKIENQVKRLTQQLENRLPQGRDLTGERFLKTLHSATKSVSVDNTEASRWQSQLLERPDLVPFPITLESNMDLMWFITQQDKIGLHIGGISEHEFTIGCGQRQLHYFQRFLSDYQTMLASKRQHTSSLFLLRSAKSIWIPSKDKGEPWNVHQLYLSCTLDTRLLTSEGTELVKQEVAAGTTKKLVTMQEKLDRTDNQDNYVKRLQSTLDRLDRPFNRPSKPLYQGQSNIIVAVSMGLQSPVTAIAIDITTQKILAYRHTKQLLGDDYRLVNRQRNLQTQQRHSSHKAQKQGLSRQCSNSELGEHLDRLFAKAIVELAQTYQAGSIALPKLDQIRLSIQSEIDTKAQQKIPGYVEGQKKYAKQIRINLHNWSYNRVSQLIINKAGQSGIAIEYGDQPARASPNVREASRNENRAGEIALSAYAKRTIP

SEQ ID NO: 87 - protein, 467 aa, unidentified
  REGION 1..467: note = Description of Unknown: MG64 effector sequence
  REGION 1..467: note = MG64-51 effector
MFPSFCSPPFLSYFSTPLNGYNRCAISYLLKNGCKINDKEENPEKFAQRRRKLEIQIQRLTEKLAARIPKGRDLTDTQWLEALFTATQTVPENETEAKFWQNSLLKKSSSVPFPVAYETNEDLVWVKNQFGRICIKFSGLSEHTFQIYCDSRQLHWFQRLLEDQKIKKNSKNQHSSALFTLRSGRISWQEGEDKGDPWNVHHLTLYCSVDTRLWTEEGTNLVKEEKAEEIAKTITQTQAKGDLNDNQQAHIKRKNSSLARINNPFPRPSQPLYQGQSHILVGVSLGLENPATIAIVDGTTSKVLTYRNIKQILGDNYKLLNKQRQQKHLLSHQRHIAQRMAASNEFGDSELGEYIDRLLAKEIIAIAQTYKAGSIVLPKLGDMREQVQSEIKAKAEQKSDLVEVQQKYAKQYRVSVHQWSYGRLIANIQCQANKAGIVIEEAKQPIRGSPQEKAKELAINAYQSRKA

SEQ ID NO: 88 - protein, 814 aa, unidentified
  REGION 1..814: note = Description of Unknown: MG64 effector sequence
  REGION 1..814: note = MG64-52 effector
MAIRTMETDVIVRTSKPRESKRSIDDLVLFDEAAWCRVCEFGYQHTLFVDTIMKQVKQLPINWLYTNQELQKQDDTTHETETKQGQNGLPEGLVKALHDALASTPQLSKMSGRFYASAIDRVEEIIESWFAAHQKLIHKLRGQRRWLAAVESDTALAESSHFSQYEIEQRAAQILAELEAKGELTAASGNQTPFNVLFKQFDETEAVLSRRAIIHLLKNINEVQVKLKQPKKGKGRNKAAQRSLTERLEAKRVEIERLEKQLLGQLPRARNLFPDQAFDQRLEALIDMPLSEATELERYYFLYFSLLLYLSGSNTYVQLEQHLLIALVLQWSKLDDLHYYRALNYSFILYAASEQQYLQLGSYLLQSIKIEAERVEAAFFAWHESITPKLQDFLREPKALPYPISFGYEDVRSWHVNQKGKIFFKLNGWGDLLFEVRCHRRQLSLIKTFCKDWQTKNNAEHKGQYSGSLMLLRSIELIWKPKKASEQKDAQLCSQCEVFQQYGGKGFWNECKLTIHWTFDTDGLTKQGSEQIRQRKLKPQLKKLQDDQAALEQKQDRLNKLEQQVAEPSQSQAQLKRLEPLKKEVQKLQEKLAKPRPKLDCLQAAKPFDRPDRPLYEGIPNIFVGVLLDLDKHLVVTVVDAMRRKRLTLRSARKVSPEGYRLLQKYFRQRQEHSQQRQQDQKAQRRSHQTESGLGQQVARLFAKGLVELAQAYKASTIVIPITGGWRERLYSQLVARAKIRCNGSKQAVDRYTKEHGERLHQWDYNRLSQAITDCAATHGIEIVLQKTVFEADVFQQSANLAIAAYDSLNSEPP

SEQ ID NO: 89 - protein, 637 aa, unidentified
  REGION 1..637: note = Description of Unknown: MG64 effector sequence
  REGION 1..637: note = MG64-53 effector
SMSQITIQCQLFADEAARKYLWQLMTDRNTPLVNELLRLIALHPDFLKWRSSGKLPTTEVTKLAKTLKTDERFNNQPAKFHISAEKTAIYTFKSWLAIQKRAQQQLEGKISWLRMLRTNEELVADCGQDLAQIRTKAQALILQYQSSEDSAESQKNFRKSLYQAYDIAEPRLGAQNERSLTRSAIAYLLKNRCHIPSDTDEDLKKFLKYRRKIENQVKRLTQQLENRLPQGRDLTGKRFLKTLDSATKSVPVDNTEASRWQSQLLERPDLVPFPITLESNMDLMWFIAQQDKIGLHIGGISEHEFTIGCGQRQLHYFQRFLSDYQTMLASKRQHTSSLFLLRSAKLIWIPSKDKGEPWNVHQLYLSCTLDTRLLTSEGTELVKQEVAAGTTKKLVTMQEKLDRTDTQDNYVKRLQSTLDRLDRPFNRPSKPSYQGQSNIIVAVSMGLQSPVTAIAIDITTQKILAYRNTKQLLGDDYRLVNRQRNLQTQQRHLSHKAQKQGLSRQCSNSELGEHLDRLFAKAIVELAQTYQAGSIALPKLDQIRLSIQSEIDTKAQQKIPGYVEGQKKYAKQIRINLHNWSYNRVSQLIINKAGQSGIAIEYGDQPARASPNVREASRNENRAREIAISAYAKRTIP

SEQ ID NO: 90 - DNA, 376 bp, synthetic construct
  misc_feature 1..376: note = Description of Artificial Sequence: Synthetic polynucleotide
  misc_feature 1..376: note = MG64-2 effector intergenic region encoding tracrRNA
  source 1..376: mol_type = other DNA
cacacaacagaaccttgataatagaatagtaaataattaatagcgccgccgttcatgcttctaggagcctctgaaaggtgacaaatgcgggttagtttggctgttgtcagacagtcttgctttctgaccctggtagctgcccaccccgaagctgctgttccttgtgaacaggaattaggtgcgcccccagtaataagggtatgggtttaccacagtggtggctactgaatcacctccgagcaaggaggaacccactttaattttttttcgtaaagccaagcgggagccaaaaccctagggggtttacgaaagtctcacgattcttacattgagtaagtttcagtgtttttgggatggttaaccctttatttacaag

SEQ ID NO: 91 - DNA, 390 bp, synthetic construct
  misc_feature 1..390: note = Description of Artificial Sequence: Synthetic polynucleotide
  misc_feature 1..390: note = MG64-4 effector intergenic region encoding tracrRNA
  source 1..390: mol_type = other DNA
tatacaaacgtttgaacctggaaaatagaataatgagaaatagcgccgcttgttcatgcgcgaaagcgtctctgaacagtgtaaatgtgggttagtttgactgtcgtgaagacggtcttgctttctgaccctggtagctgcccaccttgaagctgctgtctcttgtagacaggaatcaggtgcgcccccagtaatataggtgcgggtttaccgcagtggtggctacccaatcacctccgagcaaggaggaacccaccttaattattttttggcaaaccaaagtgggagcaatttcactgggaggttcgccaagttttcaaacaacttattttgtagaggttttgccgttttctgctaagaaaagattttcttgtccagatgcagtgataa

SEQ ID NO: 92 - DNA, 462 bp, synthetic construct
  misc_feature 1..462: note = Description of Artificial Sequence: Synthetic polynucleotide
  misc_feature 1..462: note = MG64-6 effector intergenic region encoding tracrRNA
  source 1..462: mol_type = other DNA
gatttatcagtaccgtacctggaaaattgaatataattgataacagcgccgcaggtcatgccgtcaaaagcctctgaactgtgttaaatgggggttagtttgactgttgaaagacagttgtgctttctgaccctggtagctgcccaccctgatgctgctatctttcgggataggaataaggtgcgctcccagtaataggggtgtagatgtactacagtggtggctactaaatcacctccgaccaaggaggaatccatccttaattttttattttttcgtgaacctaagcgagagcaaaatctctaggaggttcacgaaaaagctgaatccttcatggaatatgagtttcagtttgttagtgggatgatggcttctcctgaaaaggagaaatggagtaagaattttagaggtttacgaaaatgacctttaaaagctacttcaggcaagtgttacagcgctcgc

SEQ ID NO: 93 - DNA, 456 bp, synthetic construct
  misc_feature 1..456: note = Description of Artificial Sequence: Synthetic polynucleotide
  misc_feature 1..456: note = MG64-7 effector intergenic region encoding tracrRNA
  source 1..456: mol_type = other DNA
tcaaaaagaccgtaccttgaaaatataatggtaacaaacagcgccgcagttcatgcgtcttatggcgcctctgtgctgtgcaaaatgtgggttagtttgactgttggaagacagtcttgctttctgaccctggtagctgcccaccttgaagctgctatcccttgtggataggaatcaggtgcgcccccagtaatagaggtgcgggtttaccgcagtggtggctaccgaatcacctccgagcaaggaggaacccaccttaattatttttttggcatggcaaagcgggagcgattttaccgggactgatgccaaagcttcaaatctttttattgacaaggtttctagacttttgtttgtcacttgatttatttttttaattgtcaactagcaagtgattttggtagttttgccaaaagtgtctctaggaatcttgataaataaagggttttaggcgcg

SEQ ID NO: 94 - DNA, 37 bp, synthetic construct
  misc_feature 1..37: note = Description of Artificial Sequence: Synthetic oligonucleotide
  misc_feature 1..37: note = MG64-2 effector target CRISPR repeat
  source 1..37: mol_type = other DNA
gtttcaacgaccatcccaactaggggtgggttgaaag

SEQ ID NO: 95 - DNA, 37 bp, synthetic construct
  misc_feature 1..37: note = Description of Artificial Sequence: Synthetic oligonucleotide
  misc_feature 1..37: note = MG64-4 effector target CRISPR repeat
  source 1..37: mol_type = other DNA
gtttcaactttccttccagctagaggcgggttgaaag

SEQ ID NO: 96 - DNA, 37 bp, synthetic construct
  misc_feature 1..37: note = Description of Artificial Sequence: Synthetic oligonucleotide
  misc_feature 1..37: note = MG64-6 effector target CRISPR repeat
  source 1..37: mol_type = other DNA
gtttcaaccaccatctcaactagggatgggttgaaag

SEQ ID NO: 97 - DNA, 38 bp, synthetic construct
  misc_feature 1..38: note = Description of Artificial Sequence: Synthetic oligonucleotide
  misc_feature 1..38: note = MG64-7 effector target CRISPR repeat
  source 1..38: mol_type = other DNA
gtttcaacgccccttcaagctttgggcgggttgaaagc

SEQ ID NO: 98 - DNA, 37 bp, synthetic construct
  misc_feature 1..37: note = Description of Artificial Sequence: Synthetic oligonucleotide
  misc_feature 1..37: note = MG108-1 effector target CRISPR repeat
  source 1..37: mol_type = other DNA
gttgcgatcgccgctccggtggcgatggggttgaaag

SEQ ID NO: 99 - DNA, 28 bp, synthetic construct
  misc_feature 1..28: note = Description of Artificial Sequence: Synthetic oligonucleotide
  misc_feature 1..28: note = MG110-1-6 effector target CRISPR repeat
  source 1..28: mol_type = other DNA
gtgacctgccgcataggcagctggtaaa

SEQ ID NO: 100 - DNA, 27 bp, synthetic construct
  misc_feature 1..27: note = Description of Artificial Sequence: Synthetic oligonucleotide
  misc_feature 1..27: note = MG110-2-6 effector target CRISPR repeat
  source 1..27: mol_type = other DNA
tatgaactgccgcataggcagccaaga

SEQ ID NO: 101 - protein, 922 aa, unidentified
  REGION 1..922: note = Description of Unknown: MG64 transposition protein sequence
  REGION 1..922: note = MG64-13-B transposition protein
MLSSQAFNDWCQRLQINSVARAVIEQVRNAAPSRQVQGRRKNVCGSYPSRKMGVTIQFESHRNELARIHELEHDQTVLEYYDQPPPIELVYSSKTGRRTRHQYTPDFFVLRTDGVEWEECKTETELIKLAQENSNRYCQDVEGKWHCPPGEAYAQVFGFQFRVWCNSEINWVLQDNWVWLEDYLGVEVPSVSPAALQALQRHLQQTPGICVSELLAVEPTVSPDHLYHLIATEQVYVELAAARLSEPEQVRVFLDQDMAQAFHRIQGEFELVQTNKQSMLNFSIGTFLHWDEVEWEVINIGANTVGLLRSEGEVIELAKPTLENLVSRQRIVASYNSENIEKQKIINDKIKAILQEATSAEIAEANRRYDLIQACSNTSRHPSSTVRRWQNQYQKAEKIYGNGYVGLLPRYHTKGNRTMRIDDIAYQFMIEFIEQHYENPKQRRKQRVYEAFVSACEAHEPKLIPPSRITFSQAIDKRGGQAQTLKREGRRAAIAKEAFYWQLERTTPQHGSRPFEIVHLDHTQLDIELMSSLSTLAVQPLASNHRTLSQNLGRPWATFMVDAYSRRLLAVYLTYEEPSYRSCMMALRICVQRFERLPQILVVDNGVEFHSHYFEELLAYYACTKKHRPPAMARFGSVVERLFGTANTQFVHELQGNTQITRQVRQVTQSINPKRLAIWTLSDLYPALCEWAYEIYDQREHPALGQSPHDIFTQGLALGGTRDHRRVENNEVFQILTLPAPDQRRRKVQAGRGVKIHNIYYWSNAFRDPKVEGSQVEVKYDPFDASIAYAFVHHQWVKCISTYCADLQGRSERELRLVSEELRQRQRVAGCRHSLSDKELVEFLNSQHAKEGQFLQRRLQAVEQLAVLQQLDPPVDDADAESQQIEFGGDAQPSCNGNSPQTAQTVPAIVNFAVPPEFYGEF

SEQ ID NO: 102 - protein, 364 aa, unidentified
  REGION 1..364: note = Description of Unknown: MG64 transposition protein sequence
  REGION 1..364: note = MG64-13-C transposition protein
MAVQDGFPYELLDQPITDRVAYFRHYTMAHPVLLNAAQILMQTVREPAGISLVFVFGPTGVGKSTLLRRVSQKLTEAMFAALQQDQGRIPMTGIEAATPEFGQFDWKDFYLRALAAVQEPFIYPWSTSRLTTLKLRLALEHALRKRQLQVFYIDEAQNFAKVASARKLSDQTDAIKSLANLTGVQFVLTGTYEVLMLRNLSAQLCRRSIDIHFPRYRAEIPEDLQSFRGIVQTFQRHLPLPQEPNLLEQWEFCYERSLGCVGILKDWLSRALVATLEAGAKTLTPQMLEQHAWSLERCMIMLAEAKEEEKKLEVQPLQSALRIALGLETAVVEPASPPTPSVSPRRTAISAKPKRYPVGGGQHD

SEQ ID NO: 103 - protein, 267 aa, unidentified
  REGION 1..267: note = Description of Unknown: MG64 transposition protein sequence
  REGION 1..267: note = MG64-13-Q transposition protein
MIEVPSIFESWSLETFQLPRRSHLYSLSPAGLATDAIESLTSYLTRLAEAHRVSVRKLLDEKILLLLKPPRTFKYEIQSAQSWLAATPEVAKAVECLRRLTLRQDLQHLTLLPWQHQLSTPSVFHDVQPFCPVCFEEARNTDAAVYEPLLWTLEAVKVCLRHRRYLQFCCTYCGRKQPFLKLEARPGHCAHCGSWLGLELPFFVSTEPEELDWHINLAQMLGQRLQAAWQRTNQSLSKAKRSKQAQKPSLMSLLESRYELDEIKLVY

SEQ ID NO: 104 - protein, 218 aa, unidentified
  REGION 1..218: note = Description of Unknown: MG64 effector sequence
  REGION 1..218: note = MG64-54 effector
MTIITVQCRLKAPEATRRTLWELMAGRNTPLINKLIELLRGHPDLDKWRDKGKLPAGVVGELCKTLKADPLYNGQPSNAYKSAVETVEEQFESLLALHLELKEKLRIKTRRLAIFKSNAELMQLSGLTLDDMKKRAGEVLNELKEDERKKPIGTLYRKYELLFFDDPDRWAVCYLIKNGGKLPKGKEDPEKFEKSRRKLEIMVQKLTMRLDSCLPGGR

SEQ ID NO: 105 - protein, 541 aa, unidentified
  REGION 1..541: note = Description of Unknown: MG64 transposition protein sequence
  REGION 1..541: note = MG64-54-B transposition protein
MDKTNNPTLTEGLEGEAALRLEVITDLLQPCDRSTYGQRLREGAAKLGISVRSLQRLFKRYQEEGLSALMSGPRRDRGAHRIGEEWIQFIVKTYQDGNKGSKRLTPKQVAIRVRAQAHQKGQESYPSYRTVLRVLEPSIENKNKSIRSPGWRGEALSLKTRDGDALEAIRSNQVWQCDHTRADVLLVDREGRLIGRPWLTTVIDSYSRCIVGIDLSFDAPSSQKVALALRHAILPKLYGAEYKLNCEWNAYGKPECLFTDGGKDFRSNHLAEIAAQLGFILKLRDRPSEGGIVERPFKTLNQSLFSTLPGYTGPNVKERPEDAEKEATLTLRELEILIVRYIVDNYNQKQDARTGDQTRAQRWEGGIDLPEPLSERELDICLLKSTRRKIQRGGHLQFENITYRGEHLGGYEGDYMNLRYDPRDITSIWVYRQESSGDVFLCRAHAVGLQTEILSLNDAQAAAKRLRDKGRGLDNNSILQESIEREATVTKNLQRRKKEEQAYKTPTPAPEEKVELTPEVSKIEDIEHLEVWDLDELNGWG

SEQ ID NO: 106 - protein, 268 aa, unidentified
  REGION 1..268: note = Description of Unknown: MG64 transposition protein sequence
  REGION 1..268: note = MG64-54-C transposition protein
MEDKKLPIDQEWLNGEIARLNKKTVIPIEQVKSLHDWLDEKRAARQSCRVVGESRTGKTVTCQSYALRNTPRLSGNTPPNLPVVYVVPAQKCGPKDFFGAIIGALIYRAVKGSTSELRNRATEILKSCGVEMLIIDEADRLRPETFSEVRDIYDNLEIAVVLVGTDRLDAAIKRDEQVYNRFSACRRFGLLTGEDFIRAVAIWEDKILKLPVASNLTSKETLKILTRGTGGYIGKLDQILKGAAIRSLRKGHKRVETEVLKEVVKEFS

SEQ ID NO: 107 - protein, 178 aa, unidentified
  REGION 1..178: note = Description of Unknown: MG64 transposition protein sequence
  REGION 1..178: note = MG64-54-Q transposition protein
MSEDIKPWLLPVQPEPGESLSSFLGRVRQRNHLTAGALGNITRLGATIARWEKFYLNPFPSAAQLEAMAAIVGLTGEFIRDMLPPTGEAMKCEPIRLCPECYAESPYHRLQWQFQSVWKCTSHGNKLLSKCPVCSASFGIPSNWNGYCRRCGTPFELMRARSESIPALSPVDDKGYDR

SEQ ID NO: 108 - protein, 638 aa, unidentified
  REGION 1..638: note = Description of Unknown: MG108 effector sequence
  REGION 1..638: note = MG108-2 effector
MSQITIQCRLVASESTRRELWELMAEKNTPLINELLEQIGHHPDFETWREKGKLPADVVKQLAEPLKTDPRFIGQPGRFYTSAIALVKYIYKSWFAVMMQLQYQLKGKIRWLEMLKSDEELVETSGITLDNLRIKAAEILAQCTPQPDSVESQEKKSKKRKKGKKTKKSDSQPSLSKNLFDTYDSTEDIISKSAIVYLLKNGCKLTDKEEDPKEFAKRRRTTEIQIQRLTEQLSARIPKGRDLTNAKWLETLAIATENAPENETEAKSWQNKLLRKSASVPFPVSYETNEDLTWFKNNKGRLCVYFNGLSEHIFQIYCDSQHLHWFQRFLADQEVKENSKNQHSASLFTLRAGKIVWQEGEGKGEPWNINHLTLHCSVDTRLWTAEGTQQVRKEKEVEIIKFLNKNKDRSDLSKNQKANIKRQNSTLVKIANPFPRPSQPLYQGQGQILVGVSIGLESPATLAVVDAITKKVITYRSIRQLLGENYRLLNRQRRQKQSLSHQRQKAQRLADGNQLGESELGQYIDRLIAKKIVVIAAAYNAGSIVLPKLGNVRQIIQSEMDALAEQKCPEYKEGQKKYAKQYRINIHQWSYGRLIECVKTQAAKIGIVIEEAKQSIRGSPQEKAKELALAAYDSRSND

SEQ ID NO: 109 - protein, 572 aa, unidentified
  REGION 1..572: note = Description of Unknown: MG108 transposition protein sequence
  REGION 1..572: note = MG108-2-B transposition protein
MYMQDDRTRSAEIPAPSSGEGNSEVETSEVITELPLEARLKQEIIQSLLEPCDRITYGQRQREAAEKLGVSVRTVRRLVKKWEEKGLEALTQTARTDKGKHRIDEEWQEFIIKTYKEGNKGSKRITPKQVAVRVQVRAAQLGEEKYPSYRTVYRVLEPIIEQQEQTQNIRSRGWRGSRLSVKTRTGQDLSVEYSNHVWQCDHTRVDVLLVDRHSKILGRPWLTTVIDTYSRCIVGINLGFDAPSSQVVALALRHAILPKQYGLEYKLHCEWGTFGKPEHFYTDGGKDFRSNHLQQIGVQLGFVCHLRDRPSEGGIVERPFGTFNTELFSTLPGYTGSNVQERPEEAEKEACLTLRELEQLLVRYIVDKHNQSIDARMGDQSRFQRWEAGLIAVPSLMGERELDICLMKQTRRTIYRGGYLQFENLTYRGEHLAGYAGESVVLRYDPRDITTILVYRSEEGKEVFLARAYAQDLETEQLALDEAKASSRKIREAGKTVSNRSILAEVQERETFLTQKKTKKERQKEEQVQLRKAKQPLPIEPEEIEVASVRDEAESEMPEVFDYDQMREDYGW

SEQ ID NO: 110 - protein, 240 aa, unidentified
  REGION 1..240: note = Description of Unknown: MG108 transposition protein sequence
  REGION 1..240: note = MG108-2-C transposition protein
MTTQEAKTVAQQLGDFPVNDTKLQAEIQRLNRKTFVPLEQVENLHEWLEEKRQARQSGRVVGESRTGKTMGCNAYRLRHKPKQELGKPPIVPVVYFKVTQECAAKEFFGLIIQNLKYQVVKGTVPELRARAVQVLKGCRVEMLILDEADRLKPKTFAEVQDIFDDLELAVILVGTDRLDAVIKRDEQVYNRFRACHRFGKLSGEEFKRTVEIWEKKVLQLPVASNLFSKTIERGKCSQNG

SEQ ID NO: 111 - RNA, 261 nt, synthetic construct
  misc_feature 1..261: note = Description of Artificial Sequence: Synthetic polynucleotide
  misc_feature 1..261: note = MG64-6 active effector sgRNA
  source 1..261: mol_type = other RNA
  misc_difference 239..261: note = any nucleotide
ataacagcgccgcaggtcatgccgtcaaaagcctctgaactgtgttaaatgggggttagtttgactgttgaaagacagttgtgctttctgaccctggtagctgcccaccctgatgctgctatctttcgggataggaataaggtgcgctcccagtaataggggtgtagatgtactacagtggtggctactaaatcacctccgaccaaggaggaatccatccgaaaggatgggttgaaagnnnnnnnnnnnnnnnnnnnnnnn

SEQ ID NO: 112 - RNA, 263 nt, synthetic construct
  misc_feature 1..263: note = Description of Artificial Sequence: Synthetic polynucleotide
  misc_feature 1..263: note = MG64-2 effector sgRNA
  source 1..263: mol_type = other RNA
  misc_difference 241..263: note = any nucleotide
gaattaatagcgccgccgttcatgcttctaggagcctctgaaaggtgacaaatgcgggttagtttggctgttgtcagacagtcttgctttctgaccctggtagctgcccaccccgaagctgctgttccttgtgaacaggaattaggtgcgcccccagtaataagggtatgggtttaccacagtggtggctactgaatcacctccgagcaaggaggaacccactgaaaggtgggttgaaagnnnnnnnnnnnnnnnnnnnnnnn

SEQ ID NO: 113 - RNA, 265 nt, synthetic construct
  misc_feature 1..265: note = Description of Artificial Sequence: Synthetic polynucleotide
  misc_feature 1..265: note = MG64-4 effector sgRNA
  source 1..265: mol_type = other RNA
  misc_difference 243..265: note = any nucleotide
gaaatagcgccgcttgttcatgcgcgaaagcgtctctgaacagtgtaaatgtgggttagtttgactgtcgtgaagacggtcttgctttctgaccctggtagctgcccaccttgaagctgctgtctcttgtagacaggaatcaggtgcgcccccagtaatataggtgcgggtttaccgcagtggtggctacccaatcacctccgagcaaggaggaacccaccttgaaagaggcgggttgaaagnnnnnnnnnnnnnnnnnnnnnnn

SEQ ID NO: 114 - RNA, 276 nt, synthetic construct
  misc_feature 1..276: note = Description of Artificial Sequence: Synthetic polynucleotide
  misc_feature 1..276: note = MG64-7 effector sgRNA
  source 1..276: mol_type = other RNA
  misc_difference 254..276: note = any nucleotide
gtaacaaacagcgccgcagttcatgcgtcttatggcgcctctgtgctgtgcaaaatgtgggttagtttgactgttggaagacagtcttgctttctgaccctggtagctgcccaccttgaagctgctatcccttgtggataggaatcaggtgcgcccccagtaatagaggtgcgggtttaccgcagtggtggctaccgaatcacctccgagcaaggaggaacccaccttgaaaaagctttgggcgggttgaaagnnnnnnnnnnnnnnnnnnnnnnn

SEQ ID NO: 115 - RNA, 246 nt, synthetic construct
  misc_feature 1..246: note = Description of Artificial Sequence: Synthetic polynucleotide
  misc_feature 1..246: note = MG108-1 effector sgRNA 1
  source 1..246: mol_type = other RNA
  misc_difference 224..246: note = any nucleotide
gatgctaagtcgcgcctagcatcaaggagctatgtcttgattgtcttgggtgtccgccctggatgagttgaggtgtagatgcttctatcatggcagctactaaacgccccaagcaaggggaacccatctttaattttggcaaaccgaagcgggggcaaaatctccaggaggttcgccaaaacctttgaaactccttagctggaaatggcgatggggttgaaagnnnnnnnnnnnnnnnnnnnnnnn

SEQ ID NO: 116 - RNA, 274 nt, synthetic construct
  misc_feature 1..274: note = Description of Artificial Sequence: Synthetic polynucleotide
  misc_feature 1..274: note = MG108-1 effector sgRNA 2
  source 1..274: mol_type = other RNA
  misc_difference 252..274: note = any nucleotide
cttgttcgagttcgtttgaactgaacaagggtaagtatgggccagtttaattgctttccgtcccaggatagctgccagcttctaccgtaggttcgtcctgcaagtgatgctaagtcgcgcctagcatcaaggagctatgtcttgattgtcttgggtgtccgccctggatgagttgaggtgtagatgcttctatcatggcagctactaaacgccccaagcaaggggaacccatcgaaagatggggttgaaagnnnnnnnnnnnnnnnnnnnnnnn

SEQ ID NO: 117 - DNA, 550 bp, synthetic construct
  misc_feature 1..550: note = Description of Artificial Sequence: Synthetic polynucleotide
  misc_feature 1..550: note = MG64-13 effector intergenic region encoding tracrRNA
  source 1..550: mol_type = other DNA
gttgaggtggatttgtgtactgggtcaaaagatttcgctaggtttgccttttcgcgcgctgctgaagcagtgaaaagttccgacagtttaaaagttctaacacttttactgctttccgtcgggagtagttgtccgcttctgcgttgttgagaccgaagtttgtccaggtgataggcagaacgttgatgcaacaggtagatagtcgcgctctatcaggaagctgttctagttattagtgttcgtgcactaataaagaggatacagggatacatgtggttgtgtccagcaatggcacaacagggccactactcgaagccccgagcaagggttgagcctacccaaattttgtactaaaaatttggcaaaccgaagcgaggtcagtttgacccagcaccttgtcaatcagtcgaaatcttgcaccagtctgcgttttgagttggcaacagcctcacgctgaaccacttcaagcagaaagttatggcaatgtgatgacctttgccaaaataggtgtgcaaagccgactgtagcaagaatttgagcactaggagtt

SEQ ID NO: 118 - DNA, 457 bp, synthetic construct
  misc_feature 1..457: note = Description of Artificial Sequence: Synthetic polynucleotide
  misc_feature 1..457: note = MG108-2 effector intergenic region encoding tracrRNA
  source 1..457: mol_type = other DNA
tactagaaaaatttgcaccttgataatagaataatagtcacaatagcgccgtagttcatgcttgctaaagcctctgaattgcgaaaagtccgggttagtgctgtcggcagacagcgttgctttctgaccctggtagctgcccaccccgatgctgctgtcccttgcagacaggaaccaggtgcgcccccagtaataagggtgtgggtttaccacagtggtggctactgaatcacctccgagcaaggaggaatccaccttaactattttttcgtgaatccaagcgggagcaaaattcccaagggatagacgaatttggtgaaataaatagccagacttaagtttgagctttttctgggatggtcaaccctcctgttacgacagtttccaaagtaaaattttcgagttttacgagttttgtttctggaagctagtacacaaaagggttgaagcgtttaaa

SEQ ID NO: 119 - DNA, 36 bp, synthetic construct
  misc_feature 1..36: note = Description of Artificial Sequence: Synthetic oligonucleotide
  misc_feature 1..36: note = MG64-13 effector target CRISPR repeat
  source 1..36: mol_type = other DNA
tggcaattgcccttccagtgttgggtgggttgaaag

SEQ ID NO: 120 - DNA, 37 bp, synthetic construct
  misc_feature 1..37: note = Description of Artificial Sequence: Synthetic oligonucleotide
  misc_feature 1..37: note = MG108-2 effector target CRISPR repeat
  source 1..37: mol_type = other DNA
gtttcaacgaccatcccgacaaggggtgggttgaaag

SEQ ID NO: 121 - RNA, 60 nt, synthetic construct
  misc_feature 1..60: note = Description of Artificial Sequence: Synthetic oligonucleotide
  misc_feature 1..60: note = MG110-1-6 effector crRNA
  source 1..60: mol_type = other RNA
  misc_difference 9..40: note = any nucleotide
ctggtaaannnnnnnnnnnnnnnnnnnnnnnnnnnnnnnngtgacctgccgcataggcag

SEQ ID NO: 122 - RNA, 59 nt, synthetic construct
  misc_feature 1..59: note = Description of Artificial Sequence: Synthetic oligonucleotide
  misc_feature 1..59: note = MG110-2-6 effector crRNA
  source 1..59: mol_type = other RNA
  misc_difference 7..38: note = any nucleotide
ccaagannnnnnnnnnnnnnnnnnnnnnnnnnnnnnnntatgaactgccgcataggcag

SEQ ID NO: 123 - DNA, 385 bp, synthetic construct
  misc_feature 1..385: note = Description of Artificial Sequence: Synthetic polynucleotide
  misc_feature 1..385: note = MG64-6 active transposon end LE
  source 1..385: mol_type = other DNA
gggaggaaaggttaattggggggttgatttattttggttggttgtgttatatttgttattcgcgggcgattagcacagtggtagcgcacttccttcacacggaaggggtcacaggttcaaatcctgtatcgcccatttgtgtacagtgacacattaattgtcatcaatgacagattgctgtcgtggagccaaattatgtgtcgctgagacaaattaatgtcgtttaactatcagtgacaaatttttgtcgcttttcacaacaatagtgtgaaggaagtgcgcctttcaatccatcctagaaattataattccaatccctacttacctagaatggtggttgaaactgtaagattcgcgccgctaataaaactttcagcgatttgga

SEQ ID NO: 124 - DNA, 313 bp, synthetic construct
  misc_feature 1..313: note = Description of Artificial Sequence: Synthetic polynucleotide
  misc_feature 1..313: note = MG64-6 active transposon end RE
  source 1..313: mol_type = other DNA
aacacataatgctatcctagcttacaaaaaagaaagcgacaatcaatctgtcactcctcaaattctcttctttgagaacgacgacagctaaattgtcactgattgggaggacgacacttaatttgtcactaacggctagcgatctttaatcaaggaaaacagctagaatatagagaaatcaatagttttcatcagcgacaacaatttggcatcacgtcaaataattagtcactgtacaggttttatttttaatgtacagtagccctcttgcaaaagtccaagaattaggaatgttagaggaggtttgataact

SEQ ID NO: 125 - DNA, 300 bp, synthetic construct
  misc_feature 1..300: note = Description of Artificial Sequence: Synthetic polynucleotide
  misc_feature 1..300: note = MG64-2 putative transposon end LE
  source 1..300: mol_type = other DNA
ttgtaaaacctcagatgagcaaggtattgatgaataatattttccgccaaaatttacctctgtttgggcaataattgtatgatagaatttatatattagttgcttcaatagcaaaatgcgggtgtagttcagtggtagaacgtcaccttcccaaggtgaatgtcgtgggttcgagtcccatctcccgcttgtgttgttgttgtacagtgttgtcgattgccaaattatttgtccgcgttgacaaattgatgtccgtttgccaaattatttgtcatttacaaaactcgaacccacgacatt

SEQ ID NO: 126 - DNA, 400 bp, synthetic construct
  misc_feature 1..400: note = Description of Artificial Sequence: Synthetic polynucleotide
  misc_feature 1..400: note = MG64-2 putative transposon end RE
  source 1..400: mol_type = other DNA
atctacaaaattgttttatacaactatgtttgtattttagttctcaggacaaagattgtgtcaattcttcacaaaagttgtaattctcgcctttttcaaaaaaggacaaagaatttgtcaactttcctaaaccggacaactaatttgtcaaaaagctttttcaatatataaagggggtataatagctgtaacccttgcgtagagtaggttagtgagctactaggtttggcggtcaataatttggcaagtggtcaaataatttggcaatcgacaattatttatcgttggggacgaatcagtgtcacggagtcaaattatgtgtcgttgtgacagattgatgtcgtctaagcaccagtgacaaattgatgttgctttaataatttaaaatcgtcacgaaata

SEQ ID NO: 127 - DNA, 489 bp, synthetic construct
  misc_feature 1..489: note = Description of Artificial Sequence: Synthetic polynucleotide
  misc_feature 1..489: note = MG64-4 putative transposon end LE
  source 1..489: mol_type = other DNA
tttcgggttctccagttgtgctcacaaggctgcccgtttggcttcatcccgttggcaatacccatacgtttttttttacggctgtgtccaatcttccgtagtgccctcgaaatcgtgcgctgactaatctccccctcccatagctgtgccatctcgctttgggttttatccccatgctctttgacgaaagcccgaaaattctcccaatcgctgattttgcctgtctgttggcatgcttgccttggctttggcttgacatcgcccgtttcggcttggcgctcaaaccataagttaatcgtattgcgactgatattgaacagttgactcgcctcacttttcttcagaccgtccaactcaatcgcttgcattactttttgccggaagtcatcactgtaaggtttagccattgaaatgaggtagacgcaatgggtattcttctagcttacgtcctaacctgcatggctatagctatactatccacgacttgga

SEQ ID NO: 128 - DNA, 602 bp, synthetic construct
  misc_feature 1..602: note = Description of Artificial Sequence: Synthetic polynucleotide
  misc_feature 1..602: note = MG64-4 putative transposon end RE
  source 1..602: mol_type = other DNA
aagtgttcaatcaaaacacatatgtattataaagttaaaaggacatttattttgtcaatgacagtaaaaagtcacaaaaatattgatttcagaaaaaagacatatattttgtcgaatttacaatataggacggctaattcgtcaataacttcgtgctaggaattagcgatagaaaaacaataaaacccttgcatagcaaaggctttagagtaaataataagttgtcaattaatttggcaaacggtcagataatgtgacactcgacagaaatcctttctagggaaaggttttgttgtttatatatctaactttgttcgagcgctcattctcaataagcagcctagaaatgggtacagatcgagatatctagagccggaattgagataggattgagataatttaatcagggccattaagcatatggcaagggtttcggacgctacgaagaaaccacatgagataaacccaagctctcaatctttatagctcgtaagctcaacaatttcttaagtcacattgaaagcactggttctaacgatttcaatctctataggcagtcagaaaagaatccaagtgaagataaacctttgtttgtgctagtg

SEQ ID NO: 129 - DNA, 681 bp, synthetic construct
  misc_feature 1..681: note = Description of Artificial Sequence: Synthetic polynucleotide
  misc_feature 1..681: note = MG64-7 putative transposon end LE
  source 1..681: mol_type = other DNA
agttaggagtttgaagttaggagtttgaagttaccactcagcactcaatactcagcactttctactcatttccctttctttgtggtacactgcaatataggctagtcaaactatgccgggatgtagcgcagcttggtagcgcacttcgttcgggacgaaggggtcgctggttcgaatccagtcatcccgataattatttagttatttatacgacatattacacgtaatgaaagctgtacattaactaattatttgtcactgttaacagattgatgtcaaatttcacaaattgatgtcactattaacagattagtgtgcttgagctagccgttaagactcagcttttgattgtagctgaaatttatttgtttgctatgttacaggctcacccttgaaattaattataactggacatgactttacttgttcaaatacgaactgttctggtatataatgatttttgtttggggtcatacttcaaactgttaggattattttgcaatatacatttcggcagtttttgacccttcctaaaatcttgtgagaaatccgggacaactggattcgctttcaacccaccactaataattttaaattattctttccactgttgattaatccatttaatttcagctaaggcttaaaacatcaaaatattcaaagtatctcactgccaacaga

SEQ ID NO: 130 - DNA, 415 bp, synthetic construct
  misc_feature 1..415: note = Description of Artificial Sequence: Synthetic polynucleotide
  misc_feature 1..415: note = MG64-7 putative transposon end RE
  source 1..415: mol_type = other DNA
atttgtgttttataaaaacatagttgtattatactgacacctggacaaagaatttgttaaattttgcaaaatcattggtttgtttgttttttggaaaatgacaattaatgtgttatatttctgagattggacagttaatatgttaattaaggtttgaaaggctttaaatctgaaaataataaaagccccatcagaaacctttctatgacagggctttcaatatttgatagagttgtgacaataatttgttaagttgacaaataattagttaatgtacaaaatatttaatgtatagttaaaatattgtaatagtaacggttttgtctatgaattatagcaaaggacgggcaaggatgcccgttccacaagattttgatttgtagtttactttggaatttttctttttattatttaa

SEQ ID NO: 131 - DNA, 260 bp, synthetic construct
  misc_feature 1..260: note = Description of Artificial Sequence: Synthetic polynucleotide
  misc_feature 1..260: note = MG64-13 putative transposon end LE
  source 1..260: mol_type = other DNA
tgtcactgtatccataaggtgagccattgtgaaagcataagttgagccaaaaatgcttgaaatgctgatttttgcgttgcttgatttagtttaatccaaacggtttgaaagcataacgtgagccaaagttttattttgaaagcataagctgagcaaacatttgcggcaacggcttgaacagcctcaaaattgtgattagctgaaatcagaagcgatgagttccggtttcacggttgtaactcttcaggaggctgcgaaga

SEQ ID NO: 132 - DNA, 1979 bp, synthetic construct
  misc_feature 1..1979: note = Description of Artificial Sequence: Synthetic polynucleotide
  misc_feature 1..1979: note = MG64-13 putative transposon end RE
  source 1..1979: mol_type = other DNA
agagcctcactttatcgcattgccacacaagagcgcacacataaaacattaggggtaggttgaaagaggcaggggtctgcaaattaaataccaagcttgacctgaagcagcattagctttaggaactgcgatcgccatcatgcgaactgctttattaatgcagtgagcagaaattcaagtttcgacgagtaattgtcgattattagaaggagttacaaagtatggagccagaagatacggaattgagagtgagcaaaccaatccaagtgggagcaagaaatcaattctggcaaatagattgcactgtttttgatgtcagtctggtcgagaaaactactccagattattggcaaatggattttactgctcttgatgtaaatctggtcggggaaactacttcagatgaccctctttaaccctagctactgtgcgcgacacacaatcgcaaactatgttgggataccaacttacatctgaagaggtacatgggagtgcgatcgcaacactacttcatgctgccgctcgaaaaactatctatcacaatatgaattatttaatctgcgttttcatggaggcatgatggaagaacataaatttggaatcactaatgcagttatccttaattcaatatcagggtacgtcaaactcacaagaagaagacccagcaccctcaaggatattgaagagatggtgaatcgatattttcagcaaagctcaggtgactagaaacaatattcatccacgaatgcttgataagtatagcttgattctcgtctgagcgagaatcaagctggcatatatcgttagaacttcgtggacaacagcacaaaaaacaagagacaaactgctcgttaataaactcaagtttcgcattaaagctatttagaaatgggtgaagaccgttttatcttcatctatttttagtctagctcataaaccttttggaacagtacgttacaaattaattacattagcagccggggacttagcgctacagataaacgtgaatctgggataagctgaaacgcatttaacccgagataaagttagctcatctaggtatagacacactagtccaattaatttggaggctttcttattcaggttatacatactgaaactcttttcctctctatctgccaagcaccgcaactaatttgatagaaggcaaccaggaaatttattggataaattggcgcggtaatttgacgatgtgttagggcagtgatgcactgcttcttgtgaagaagcgtttaaagagtaatttgtctctcatatttcttgctcaacgcagcaaatgtgatgctagtgcaaaatagcgtgacgggtgacacttactaaattgagtcagccgagcatcggatggtgatatggagcgacagcaaaccagagcatcggttgccaatccagcaatctagctgactaagcagtgcaagtaatttgacagcagagcaaaacagcgtgactgttgacaaggagaaatttcatttctagatcgtgccattaatttgcgaatttaggtgaattttgtgctatcaatttgcgaacctagaatttgtgccattgatttaagaacaaaggatcgtgcagtagaccgccagatcggttgcaatgcactaggttcgattccttcaatgtgctttcagatgaacaagcaagtatctgtgccactgatttgcgaatcggtcgaagatttgtgccacagatttacaaatgactattcgaccctaacaagccgattgtgccagcaatttgcgaatcgtgccatattttttgattattcacacgtgcgactttcccaaaagtcagccaaaaatcgctcaaaacgacattagcgtgcgacaagcgacatttatcgtgcgaccgtacatttgcttttacaaaggcggcacccagattcgaactgggggtggaggttttgcagacctctgccttaccacttggcgatgccgccgaattgcatcatggacattgtatca

SEQ ID NO: 133 - DNA, 1444 bp, synthetic construct
  misc_feature 1..1444: note = Description of Artificial Sequence: Synthetic polynucleotide
  misc_feature 1..1444: note = MG64-54 putative transposon end LE
  source 1..1444: mol_type = other DNA
ccgccggagggtcgagcgacaaactgagcaaaagtagtgataaattttgtctaaaatcaagcgacctcattctctgtacagtgccagattaaatgacgtgatgccaaattagtgtcgtcggttgatacccttgctacatcaacactctacagcctcataaccattcttagacctatcactttagtgccaaattgtgagtcgtttaatcaaataatgacagattaatcgtctttaaagaattaatctcagtgacgaattaactgtcattatccgacgatagccccaagataatatcatgttgcctctaacatgaatatgtaatggggagaatatcctaaaccattagtatcctttcctaaataaacactttcattcgcgcgaagagttatagcgccgttattgccgataaactcaagcgccgttattgccgataaactcaatggtgttattctacaacggcggtcaattaatatttatagtgaaactcctccatttttagtaaaaaatagcaacgtaacaacatagggctatcgtttattataaatacgtaattcccgaaaattagtgggaaggtcacaccttccatccgttccatatatctgagagctgaattgatttgaattcatcaaactccaagtcgcatacattctagtagtacagccatttcttctatcctgacgtgtataagttacagaggcttgattataactcttatggcggcctttaatttgatgatcgaaatctcctgaatatagttcagcattaatattaactccatcatcactagaattaataactactgcgtcctgttctgaagcttgccacataccattaaatgctccaggtgctctattttgagcaaaagcagaagacaatccagaaatagctattacgcaaatagaagaagcaagtacaagtacgagttttttaggtctcatggtttttaattctaaaagctagtcaatttgtgagattgaagggtgttaaactaaagcagaggtagctacaaataatgtagaagctacgaaattacgtatgaaacttttttcaaatccagcgtggtcattatggatagtgatttttacgcctatagcttcttattctaaaggaatttcctattaacgatcgccgctattgtagcagtggcataattgtcacactaaggattgactcccaaccgtataattaaacactatttgaaaggtaggggcagattttgatgtttgctactccacggtataagttttcttcggggagagctaaaacgtcctttattcgccgatgcaggcgaaaccatagagttttgaattacagggtggaagacccaaacaaaacgtgccgctcatgttctgttgtctccgccagtgctaaatgccaaactaattgtcgcattatcttagtatgttgctaggataacaatatgttttattaaaatttttattta

SEQ ID NO: 134 - DNA, 307 bp, synthetic construct
  misc_feature 1..307: note = Description of Artificial Sequence: Synthetic polynucleotide
  misc_feature 1..307: note = MG108-1 putative transposon end LE
  source 1..307: mol_type = other DNA
tgtccattaaagaattatttgtcgcctttaaagaattaatgtccaatttcaggaattttggatgtttcaggctttggcaagaaatctcttgctcaaaattttggtttaaagaattaatgtccaaaatttcgacatacttgcgttgaaatacatcactcatcgttagagtgcgatcgcagcgctcctttcaacccatctacattcacacaaaagcactcttaccaaagactcttactctttcagtgcaatcaaccttggaagggcgattacaaagcagtcttccaaattcagcgtagtattgacattc

SEQ ID NO: 135 - DNA, 619 bp, synthetic construct
  misc_feature 1..619: note = Description of Artificial Sequence: Synthetic polynucleotide
  misc_feature 1..619: note = MG108-1 putative transposon end RE
  source 1..619: mol_type = other DNA
ggtgtgagcagttaacaaccatggcttaaataatgacactttttggtttaaatattcaaccagcgaatagtaaaatttatgacaaattttgctttaaacaaagctatggctattttggtgaagtgaatcagcctgaagatcaatagcagcaagggttgcatcacctagcagtgtttagagccaagatgacaataattctttaaagtcggtcaaacaattctttaatggacaccatgtgtgatgtacactaacgctcatctgtcactctaggcagatgtccattaaggagctatttgtcagttttaaacagctcttgtcaaaaaattttagtactcaacgtattttcaagggttttaagcgacaccccttgatttgctgcttttttgccctgaaattccagttttaaggagatgttgtcacagatgattcatgccttgctaccctgtatcggtatatggacagagtcattacagacgatcgcctgtagtgcgatcgcagcgctcctttctatccacattaattcacatgttttaacgctcaccactaatcgactgctgagcggttgctgtaatagcgtcgagacgtatgtaggccgaaatcgatcgcatccgcaacagcg

SEQ ID NO: 136 - DNA, 709 bp, synthetic construct
  misc_feature 1..709: note = Description of Artificial Sequence: Synthetic polynucleotide
  misc_feature 1..709: note = MG110-1-6 putative transposon end LE
  source 1..709: mol_type = other DNA
tattgttatccctatttattgttattagacaaattgtttatttaaacaacaataaatagataccatcaaaatacaatttgtacagtgataaaatcgaaaaaatagataacaatttgttatatgcagcgcgaaaactggaatagagaactgagcggcgtggtttaatatatgagcggcaatagtggaagtttaagggggaatgtatcgctgacgggtatagcttgttgattgaagcataaattgacaaattcacaaacgaaaaatcaatgagttacgcttctctttgaaccataagatggcaaaaaattaaaccataagctgacaaatttcttagtgaacgtgttacaccacccaaaacgtggtaagccactgttgtaaaattacaagatatctcatgtacttcatagttacgaagtcggtttagctaagggcgagtcactcacgaccagttcaacagtgttacgggcactgtttaccagcgacctatgacgaagaccttcgatactttcaataatactactgtagagcatttcaaaggccatcgaaatatagaggtgtagttatattttaagcgtctcagagcaaaaaagccctctccatagcgctttcacattttcatccattctcgatctcgacaatgaaacttaactcccaaatcaacgagccagcggagaggagcaatttagctagctccgcgtgcattggcttg

SEQ ID NO: 137 - DNA, 479 bp, synthetic construct
  misc_feature 1..479: note = Description of Artificial Sequence: Synthetic polynucleotide
  misc_feature 1..479: note = MG110-1-6 putative transposon end RE
  source 1..479: mol_type = other DNA
aaccgagacctgtaagttgtaagtaaataacaacttaaatataggttatgccagctattactgcaaatttgacaacttatggttcaaatttgtcactttatgcttcaatttacatagctttaagttggtggcatctctgccagcctcaccccggcacccgagtctaccgctgtggctgcttccttccggacctgaccagattcacggtttatcgttgcggggggaccagcagagacaccatagaggaggtctcgttgaccttggtggggcattatacgcactctgtttcgatgtgcaacctacgctatggggtttattatcttttttctattagtgagaatactttaacgaaattcgctggttcgccgctcccgttgctgaagcttttagcgctctcggctatacttgactaactttatcggtttttgccgctgtctctgtatcacagagccggcgatgtattttatgggagcgcgcag

SEQ ID NO: 138 - DNA, 474 bp, synthetic construct
  misc_feature 1..474: note = Description of Artificial Sequence: Synthetic polynucleotide
  misc_feature 1..474: note = MG110-2-6 putative transposon end LE
  source 1..474: mol_type = other DNA
tgaggttgcctccagtacgtccacaccacgtggatgaccagacaggctttgaggttaactaattgatgttgttacaaccataagttgacataacggccataataaaatcaacaggttacaaaacgatttttacccataagttgacataaaaatgaagcataagctgacatagagtgacaagaccacacatggacacaaaatcggcgttgtaagtttacaagatcgacttgtagcatccactataggcaaacacacctacggcagccatcctctcattaagtgcaagtggcactatcgaacacaacgctcaccatttttgcgtagggcaaagtcgatccgccccgaattttctagaccatccgaacctttggctagcaggctacatgaatttctccatgtgccggatatcgagccattgtctgcataattataaaacgctccctattgcttgaaacgacttgcattgttttgagg

SEQ ID NO: 139 - DNA, 334 bp, synthetic construct
  misc_feature 1..334: note = Description of Artificial Sequence: Synthetic polynucleotide
  misc_feature 1..334: note = MG110-2-6 putative transposon end RE
  source 1..334: mol_type = other DNA
agcgcctccaacctgtagttgcttttctacaggtttaggctatgtcaacttatgatgcaagtttatgtcagcttttggttccactatgtcaacttatggttccaacgacaattgatttgccagaaatggtgggggctctgccagccacatcccggcactcgaaggactcgccttggctgctaccttccggtcctgaccaggtcgacgagattccgatgcgggaggaccaacagagcccccataacaacgccggcgagtctacctagcctgaccggcaattgcaatcgctttaccggtcagctgagttcaagtgtctgaaaactcgacaagtagc

SEQ ID NO: 140 - protein, 16 aa, synthetic construct
  REGION 1..16: note = Description of Artificial Sequence: Synthetic peptide
  REGION 1..16: note = Nucleoplasmin NLS
KRPAATKKAGQAKKKK

SEQ ID NO: 141 - protein, 19 aa, synthetic construct
  REGION 1..19: note = Description of Artificial Sequence: Synthetic peptide
  REGION 1..19: note = SV40 2x NLS
PKKKRKVDGSPKKKRKVDS

SEQ ID NO: 142 - protein, 19 aa, synthetic construct
  REGION 1..19: note = Description of Artificial Sequence: Synthetic peptide
  REGION 1..19: note = P2A
ATNFSLLKQAGDVEENPGP

SEQ ID NO: 143 - protein, 18 aa, synthetic construct
  REGION 1..18: note = Description of Artificial Sequence: Synthetic peptide
  REGION 1..18: note = T2A
EGRGSLLTCGDVEENPGP

SEQ ID NO: 144 - protein, 9 aa, synthetic construct
  REGION 1..9: note = Description of Artificial Sequence: Synthetic peptide
  REGION 1..9: note = HA
YPYDVPDYA

SEQ ID NO: 145 - protein, 10 aa, synthetic construct
  REGION 1..10: note = Description of Artificial Sequence: Synthetic peptide
  REGION 1..10: note = Myc
EQKLISEEDL

SEQ ID NO: 146 - protein, 8 aa, synthetic construct
  REGION 1..8: note = Description of Artificial Sequence: Synthetic peptide
  REGION 1..8: note = FLAG
DYKDDDDK

SEQ ID NO: 147 - protein, 640 aa, unidentified
  REGION 1..640: note = Description of Unknown: MG64 effector sequence
  REGION 1..640: note = MG64-56 effector
MSVITIQCRLVADDKALRHLWELMAEKNTPLVNELLDRLGKHTDFEAWVQAGKVPKTTIKALCDSLKTQEPFIGQPGRFYTSATTLVAYIYKSWLALHKRRQRKIEGKERWLEMLKSDVELEQESNSSLELIRTIATEILSKFSASSTDGINQKSKGKKSKKLKKDKADEPMSIKPGVLFEAYQKTEDILRRSALVYLIKNNCQVNFAEEDPDKYAKMRRKKEIEIERLKEQLKSRVPKGRDLTGKKWLETLEKAVNSIPQDENEAKSWQAGLLRKSSTVPFPVAYETNEDMHWEISDKGRIFVSFNGLSKLKLEVYCDQRHLPWFQRFVEDQETKRKGKNQHSSGLFTLRSGRLSWLKQEGKGEPWSVNRLILFCSVDTRMWTVEGTQQVAIEKIADVEQNLTKAKEKGELNSNQQAFVTRQQSTLAKINTPFPRPSKPLYEGKSHILVGVSLGLENPATVAVFDAVNNKVLAYRSVKQLLGNNYNLLNRQQQQKQRLSHDRHKAQKDFARNDFGESELGQYVDRLLAKEIVAIAVTYFAGSIVLPKLGDMREIIQSEVQARAEKKIPGFKEGQQKYAKEYRKQVHNWSYGRLIENIQSQAAKVGILIETGQQPIRGSPQEQARDLALFAYQCRIASSI

SEQ ID NO: 148 - protein, 579 aa, unidentified
  REGION 1..579: note = Description of Unknown: MG64 transposition protein sequence
  REGION 1..579: note = MG64-56-B transposition protein
MGETLNSNEVDESLVLYDGSDEVDEISESEDTKQNNVIVTELSEEAKLRMEVLQSLIEPCDRKTYGIKLKQAAEKLGKTVRTVQRLVKKYQEQGLSGVTEVERSDKGGYRIDDDWQDFIVKTYKEGNKGGRKMTPAQVAIRVQVRAGQLGLEKYPCHMTVYRVLNPIIERKEQKQKVRNIGWRGSRVSHQTRDGQTLDVHHSNHVWQCDHTKLDVMLVDQYGETLARPWLTKITDSYSRCIMGIHLGFDAPSSLVVALAMRHAMLRKQYSSEYKLHCEWGTYGVPENLFTDGGKDFRSEHLKQIGFQLGFECHLRDRPPEGGIEERGFGTINTDFLSGFYGYLGSNVQERAEGAEEEACITLRELHLLIVRYIVDNYNQRIDARSGNQTRFQRWEAGLPALPNLVNERELDICLMKKTRRSIYKGGYVSFENIMYRGDYLSAYAGESVLLRYDPRDISTVFVYRQDSGKEVLLSQAHAIDLETEQISLEETKAASRKIRNAGKQLSNKSILAEVQDRDTFIKQKKKSHKERKKEEQAQVNFVKPPQTNEPVETVEEIPQPQKRRPRVFDYEQLRKDYDD

SEQ ID NO: 149 - protein, 278 aa, unidentified
  REGION 1..278: note = Description of Unknown: MG64 transposition protein sequence
  REGION 1..278: note = MG64-56-C transposition protein
MAEDYLRKWVQNLWGDDPIPEELLPIIERLITPSVVELEHIQKIHDWLDSLRLSKQCGRIVAPPRAGKSVTCDVYKLLNKPQKRTGKRDIVPVLYMQVPGECSAGELLTLILESLKYDAISGKLTDLRRRVLRLLKESKVEMLVIDEANFLKLNTFSEIARIYDLLKISIVLVGTDGLDNLIKKEPYIHDRFIECYRLPLVSEKKFPEFVQIWEDEVLCLPVPSNLTKRETLMPLYQKTSGKIGLVDRVLRRAAILSLRKGLKNIDKATLDEVLEWFE

SEQ ID NO: 150 - protein, 176 aa, unidentified
  REGION 1..176: note = Description of Unknown: MG64 transposition protein sequence
  REGION 1..176: note = MG64-56-Q transposition protein
MEIPAEQPRFFQVEPLEGESLSHFLGRFRRENYLTATQLGKLTGIGAVISRWEKFYLNPFPTPQELEALAAVVEVKVDRLIEMLPPRGVTMKPRPIRLCSACYQESPCHRVEWQFKDVMVCDCLRHCPLNNRHQLALLTKCTNCETPFPIPADWVQGECPHCFLPFTKMARRQKRY

SEQ ID NO: 151 - DNA, 485 bp, synthetic construct
  misc_feature 1..485: note = Description of Artificial Sequence: Synthetic polynucleotide
  misc_feature 1..485: note = MG64-56 effector intergenic region encoding tracrRNA
  source 1..485: mol_type = other DNA
caaaataccgaaccttgaaaacttaatatgaaagtaacagcgccgcagttcatgctcttctgagtctctgtactgtgataaatctgggttagtttaacggttgaaagaccgttttgctttctgaccctggtagctgctcgctcttgatgctgctgtcttttgacaggataggtgcgctcccagcaataaagagttaaagctgataaagcttgagccgttgtaaaacggtggggtttacctcagtggtggctactgaatcacccccttcgtcgggggaaccctcctaaatattttttttggcgtgtcaaagcgggggcaaaaatcctggagtcccgccaaaatctcaaaacctttgtcctatcttgacttgataaactagcatgtcagttaatttagttttttgatgtcaagtaggagatgcttttaggcagtcctgccaaagatgtgtatggaaagctctaatagcaagggttctagacggatcg

SEQ ID NO: 152 - DNA, 37 bp, synthetic construct
  misc_feature 1..37: note = Description of Artificial Sequence: Synthetic oligonucleotide
  misc_feature 1..37: note = MG64-56 effector target CRISPR repeat
  source 1..37: mol_type = other DNA
gtttcaacaaccatcccagctaggggtgggttgaaag

SEQ ID NO: 153 - DNA, 595 bp, synthetic construct
  misc_feature 1..595: note = Description of Artificial Sequence: Synthetic polynucleotide
  misc_feature 1..595: note = MG64-56 putative transposon end LE
  source 1..595: mol_type = other DNA
tatgcgacttgagcgatttgtgtaagttttgtgttgcacaaaccatcctaagcgacattagtttgcaaaaaacgacattaatttacgaatcgcgacctttaatttgcgaatatacaacagattttgtcgattaactaattatttgtcgtcttaacaaattaatgtcgcccaaatcttcaagactataatccttatgtatcaaaggttatagccttttgaacttatattggctatcatcaaatatttaactaattaagtgtcgtcttttaattaattaacattttaaatgtcgttttttcaaaaaacacctttccaaatttttcttttgctcataacaaaataactgtcgtcttttggaagtgagtgaaaaatataaaattaaatgtcgctttttggaacaaagtagtatgatatttattaggcaatagtagctatgtaacaacaaaaacatagttagattgaagtcttcttttttgtctctagctacgaagtcattacccttgctgcgattaaatttagacgcaagctaatttcgctcttagacttgctgtaccgtattgcctaaccaactagtttcaagcgatgaagtttgttt

SEQ ID NO: 154 - DNA, 493 bp, synthetic construct
  misc_feature 1..493: note = Description of Artificial Sequence: Synthetic polynucleotide
  misc_feature 1..493: note = MG64-56 putative transposon end RE
  source 1..493: mol_type = other DNA
taagttctattaactccaagttttaataattgcatggcaataacaatcctttttagaaaggatttaagagggttgaaaggaatgtcaccttcccaagaatacttttcaaaagctattttgggttagggaagaataatcacagataactaatatgcacaagtaagtctaaaatagggataagtctgtcgattagtccaatagcaaggcatcttgttagacgacattaatttgttaacgttagttggaactaattcgacgacattaattcgttaacagcgacattaatttgttaatgacgacattaatctgttaacgacgacattaatctgttaacgacgacaaataatctgttaattgacagatttgaaagcgggtgatgggactcgaacccacgacgttcaccttgggaaggtgacattctaccactgaattacacccgcaaatggagtttaggctcaataaagctaacccccattataacacgatttgccca

SEQ ID NO: 155 - DNA, 568 bp, synthetic construct
  misc_feature 1..568: note = Description of Artificial Sequence: Synthetic polynucleotide
  misc_feature 1..568: note = MG64-2 active transposon end RE
  source 1..568: mol_type = other DNA
cctcgtgatacgcctatttttataggttaatgtcatgataataatggtttcttagacgtcaggtggcacttttcggggaaatgtgttctatctacaaaattgttttatacaactatgtttgtattttagttctcaggacaaagattgtgtcaattcttcacaaaagttgtaattctcgcctttttcaaaaaaggacaaagaatttgtcaactttcctaaaccggacaactaatttgtcaaaaagctttttcaatatataaagggggtataatagctgtaacccttgcgtagagtaggttagtgagctactaggtttggcggtcaataatttggcaagtggtcaaataatttggcaatcgacaattatttatcgttggggacgaatcagtgtcacggagtcaaattatgtgtcgttgtgacagattgatgtcgtctaagcaccagtgacaaattgatgttgctttaataatttaaaatcgtcacgaaataagcctaacgtgagttttcgttccactgagcgtcagaccaccgttgatgataccgctgccttactgggtgcattagccag

SEQ ID NO: 156 - DNA, 227 bp, synthetic construct
  misc_feature 1..227: note = Description of Artificial Sequence: Synthetic polynucleotide
  misc_feature 1..227: note = MG64-4 effector putative tracrRNA
  source 1..227: mol_type = other DNA
aatagcgccgcttgttcatgcgcgaaagcgtctctgaacagtgtaaatgtgggttagtttgactgtcgtgaagacggtcttgctttctgaccctggtagctgcccaccttgaagctgctgtctcttgtagacaggaatcaggtgcgcccccagtaatataggtgcgggtttaccgcagtggtggctacccaatcacctccgagcaaggaggaacccaccttaattat

SEQ ID NO: 157 - DNA, 228 bp, synthetic construct
  misc_feature 1..228: note = Description of Artificial Sequence: Synthetic polynucleotide
  misc_feature 1..228: note = MG64-7 effector putative tracrRNA
  source 1..228: mol_type = other DNA
aacagcgccgcagttcatgcgtcttatggcgcctctgtgctgtgcaaaatgtgggttagtttgactgttggaagacagtcttgctttctgaccctggtagctgcccaccttgaagctgctatcccttgtggataggaatcaggtgcgcccccagtaatagaggtgcgggtttaccgcagtggtggctaccgaatcacctccgagcaaggaggaacccaccttaattat

SEQ ID NO: 158 - DNA, 328 bp, synthetic construct
  misc_feature 1..328: note = Description of Artificial Sequence: Synthetic polynucleotide
  misc_feature 1..328: note = MG64-9 effector putative tracrRNA
  source 1..328: mol_type = other DNA
gctgaagttagtctctggatactgaagtcagtgtctagggactgaagtaaggggagctttacccgcaagggatactttcgaccccctgtagctgcccgctcctggtggggtgccctgacacccgcctcattacagcaatgtatgactgtctgggctaatgaaagaaggattagggacgcaggttcacgacctacttcaatcatagtctgtgcaacccagataagtgagtatgacccgcaagggtctaaacgcctttagtagaatggttcttccagtttgttaagggcagggcttggttctccaaggtggctacgaattcttctcgatt

SEQ ID NO: 159 - DNA, 187 bp, synthetic construct
  misc_feature 1..187: note = Description of Artificial Sequence: Synthetic polynucleotide
  misc_feature 1..187: note = MG64-10 effector putative tracrRNA
  source 1..187: mol_type = other DNA
ggtagcgctggagccacatagttcataagctcacgcttcttggacttcctgtgttctctaaaacgggttctgttttacccttaccaagggatactttcagatccgagtagctgcaagctcatggcggagtgtcccctgacgctttgccaccgtcatagcgatgtgatggccgtctggcgtatgaacg

SEQ ID NO: 160 - DNA, 326 bp, synthetic construct
  misc_feature 1..326: note = Description of Artificial Sequence: Synthetic polynucleotide
  misc_feature 1..326: note = MG64-12 effector putative tracrRNA
  source 1..326: mol_type = other DNA
aatagcgcccctaatcaaatcgaaggcgtttgaggacggggaaaatgggtaagtttccatcgaaaggtggttcttttcagccctatgtagtcccactcccttgtggagtgttagcatctgaggtgcctagcacagcaaagtctctcaggagacggagtcaaagctggggaacaagtattacgaacgaatctcgcgattctattagtatacgtaggtcgctcccatgcaacaagatgctcatctctcagcaatgagggtagggaacgtatcccaaacactacatgaatagctcgtcgggcttgcccggtgagtatccaccaagcctt

SEQ ID NO: 161 - DNA, 311 bp, synthetic construct
  misc_feature 1..311: note = Description of Artificial Sequence: Synthetic polynucleotide
  misc_feature 1..311: note = MG64-13 effector putative tracrRNA
  source 1..311: mol_type = other DNA
cgctaggtttgccttttcgcgcgctgctgaagcagtgaaaagttccgacagtttaaaagttctaacacttttactgctttccgtcgggagtagttgtccgcttctgcgttgttgagaccgaagtttgtccaggtgataggcagaacgttgatgcaacaggtagatagtcgcgctctatcaggaagctgttctagttattagtgttcgtgcactaataaagaggatacagggatacatgtggttgtgtccagcaatggcacaacagggccactactcgaagccccgagcaagggttgagcctacccaaattt

SEQ ID NO: 162 - DNA, 342 bp, synthetic construct
  misc_feature 1..342: note = Description of Artificial Sequence: Synthetic polynucleotide
  misc_feature 1..342: note = MG64-14 effector putative tracrRNA
  source 1..342: mol_type = other DNA
aacagcgccgcagtttaagctctatagccgctgaactgtgaaaaatgtgggtcagtttggtcgttgcaagacgatcgtgctttccgaccctagtaactgtccgctcactgactgccatcctggggcaaatcttcaaattttgtgtatttgtgtggggatggaaagctgcattagtcgattctcttcctccaatgtagcgtaggtgcgcacccagcagaagtgagttaagccttcacaatgtggaggtacagaagcatcatctctccattttttggtgtagatggtgtgactgaagtggtagttaccgaatcgcccctgatcaagggggagccctccataatt

SEQ ID NO: 163 - DNA, 297 bp, synthetic construct
  misc_feature 1..297: note = Description of Artificial Sequence: Synthetic polynucleotide
  misc_feature 1..297: note = MG64-15 effector putative tracrRNA
  source 1..297: mol_type = other DNA
aatgacgtttgtttctgtgcgcgctgctgaagcagtgaaaaatacggacagtctaatttgctttccgtccggagtagttgtccgcttctgcgttgttggaaccgaagtttgtccaggtgataggcggagtgtttcggcaacagatggagagtcgcacctctatcagggggcagttttagtggttagtgtttagcgcactatcactaggatacagggatacatgtggtagtgaccagagatggcactgcgggaccgctactcgaagccccgaacaagggttgagcttacccaaatctt

SEQ ID NO: 164 - DNA, 226 bp, synthetic construct
  misc_feature 1..226: note = Description of Artificial Sequence: Synthetic polynucleotide
  misc_feature 1..226: note = MG64-16 effector putative tracrRNA
  source 1..226: mol_type = other DNA
aatagcgccgcagtttaagctcagcaagcctctggactgcgaaaagtatggggtagtttgaccgtcggtaaacggttgtgctttctgcccctggcgactgcccaccccgatgctgtcgatttcttaactgggaatcgagatgaggtgcgcccccagcaaaagggaacgggtttactggagtggtggtcgccgaatcacccccgagcaagggggactcgtcctttgc

SEQ ID NO: 165 - DNA, 227 bp, synthetic construct
  misc_feature 1..227: note = Description of Artificial Sequence: Synthetic polynucleotide
  misc_feature 1..227: note = MG64-17 effector putative tracrRNA
  source 1..227: mol_type = other DNA
aatagcgccgcagtttaagctcaatgagcctcttgactgcgaacagtatggggtagtttgaccggcggtaaccggttgtgctttctgcccctggcgactgcccaccccgatgctgtcgatttctcaaccgggaatcgagaatcaggtgcgcccccagcaagagggaacgggtttactggagtggtggtcgccgaatcacccccgagcaagggggactcgtcctttgc

SEQ ID NO: 166 - DNA, 227 bp, synthetic construct
  misc_feature 1..227: note = Description of Artificial Sequence: Synthetic polynucleotide
  misc_feature 1..227: note = MG64-19 effector putative tracrRNA
  source 1..227: mol_type = other DNA
aatagcgccgcagtttaagctcagcaagcctctggactgcgaaaagtatggggtagtttgaccgtcggtaaacggttgtgctttctgcccctggcgactgcccaccccgatgctgtcgatttctcaaccgggaatcgagaatcaggtgcgcccccagcaagagggaacgggtttactggagtggtggtcgccgaatcacccccgagcaagggggactcgtcctttgc

SEQ ID NO: 167 - DNA, 211 bp, synthetic construct
  misc_feature 1..211: note = Description of Artificial Sequence: Synthetic polynucleotide
  misc_feature 1..211: note = MG64-20 effector putative tracrRNA
  source 1..211: mol_type = other DNA
aatagcgccgcagtttaagctcaatgagcctcttgactgcgaacagtatggggtagtttgaccggcggtaaccggttgtgctttctgcccctggcgactgcccaccccgatgctgtcgatttctcaaccgggaatcgagaatcaggtgcgcccccagcaagagggaacgggtttactggagtggtggtcgccgaatcacccccgagcaagg

SEQ ID NO: 168 - DNA, 222 bp, synthetic construct
  misc_feature 1..222: note = Description of Artificial Sequence: Synthetic polynucleotide
  misc_feature 1..222: note = MG64-21 effector putative tracrRNA
  source 1..222: mol_type = other DNA
aatagcgccgcagttcatgcttctttgaagcctctgtgctgtgcaaaatgtgggttagtttggctgttgaagaaacagccttgctttctgaccctggtagctgtccaccctgaagctgctatcccctgtggataggataggtgcgcccccagcaataggggagcgggtataccgcagtggtggctactgaatcacctccaagcaaggaggaatccactttat

SEQ ID NO: 169 - DNA, 222 bp, synthetic construct; identical to SEQ ID NO: 168
  misc_feature 1..222: note = Description of Artificial Sequence: Synthetic polynucleotide
  misc_feature 1..222: note = MG64-22 effector putative tracrRNA
  source 1..222: mol_type = other DNA

SEQ ID NO: 170 - DNA, 248 bp, synthetic construct
  misc_feature 1..248: note = Description of Artificial Sequence: Synthetic polynucleotide
  misc_feature 1..248: note = MG64-24 effector putative tracrRNA
  source 1..248: mol_type = other DNA
aaaagcgccgtagaacatgctcacgcctctgttctgcgaaaaattagggtttgtttggctgtctgacagcagtcttactttctgtccctagaatctgaccactccgatgctgctgttgtaagtgaacttgattgagctcggacaccatgcaacaggataaggggcgcacccagcaagagaggacggacttaccgtagtgttggcttctgaagcaactccgaccaaggagtagtccatgcattcatcat

SEQ ID NO: 171 - DNA, 277 bp, synthetic construct
  misc_feature 1..277: note = Description of Artificial Sequence: Synthetic polynucleotide
  misc_feature 1..277: note = MG64-25 effector putative tracrRNA
  source 1..277: mol_type = other DNA
aatcgcgccgcacattcatgttccttgagaacctctgaattgcgaaagtgtgggctagtttgttcgcttgatgcgaatgtgctttctggccctggtagctgtccgccctgatgctgatttctacgggtaactgtaggaatgattaactcgttctatagacaggtttcgtgctttctattgtaacggggtcggtgcgctcccagcaataggggtgtgggtctactacagtgatggctactgaatcacctccgagcaaggaggaatccaccttaacttt

SEQ ID NO: 172 - DNA, 226 bp, synthetic construct
  misc_feature 1..226: note = Description of Artificial Sequence: Synthetic polynucleotide
  misc_feature 1..226: note = MG64-27 effector putative tracrRNA
  source 1..226: mol_type = other DNA
aatagcgccgcagtttaagctcagcaagcctctggactgcgaaaagtatggggtagtttgaccgtcggtaaacggttgtgctttctgcccctggcgactgcccaccccgatgctgtcgatttcttaactgggaatcgagatgaggtgcgcccccagcaagagggaacgggtttactggagtggtggtcgccgaatcacccccgagcaagggggactcgtcctttgc

SEQ ID NO: 173 - DNA, 226 bp, synthetic construct; identical to SEQ ID NO: 172
  misc_feature 1..226: note = Description of Artificial Sequence: Synthetic polynucleotide
  misc_feature 1..226: note = MG64-28 effector putative tracrRNA
  source 1..226: mol_type = other DNA

SEQ ID NO: 174 - DNA, 255 bp, synthetic construct
  misc_feature 1..255: note = Description of Artificial Sequence: Synthetic polynucleotide
  misc_feature 1..255: note = MG64-29 effector putative tracrRNA
  source 1..255: mol_type = other DNA
tagcgcacgaacctgactagcgtcattacgatgcgagttctggaaatgggacagtttcattgctttccgtccctggcactgcctgcttactcacgaccacaaggagacaattcagcgctgtgagttcgcgattctctactgtttatgacaaactaacgtttgtctggcagtacagcaagattgtgtatcagaccatgtttggcgaagatacatggcaggccgaatcgccaatgaaaccagcggacttcccctaat

SEQ ID NO: 175 - DNA, 226 bp, synthetic construct
  misc_feature 1..226: note = Description of Artificial Sequence: Synthetic polynucleotide
  misc_feature 1..226: note = MG64-32 effector putative tracrRNA
  source 1..226: mol_type = other DNA
aatagcgccgcagtttaagctcaatgagcctcttgactgcgaacagtatggggtagtttgaccggcggtaaccggttgtgctttctgcccctggcgactgcccaccccgatgctgtcgatttcttaactgggaatcgagatgaggtgcgcccccagcaagagggaacgggtttactggagtggtggtcgccgaatcacccccgagcaagggggactcgtcctttgc

SEQ ID NO: 176 - DNA, 203 bp, synthetic construct
  misc_feature 1..203: note = Description of Artificial Sequence: Synthetic polynucleotide
  misc_feature 1..203: note = MG64-44 effector putative tracrRNA
  source 1..203: mol_type = other DNA
tctagcgccgcagctcatgtcagcaatggccaatgtgttgtgctaaatgcgagctagtttgactgcctgctaagcagtcttgctttctggctcaggtgactatccacccaaaggtcgttggtgcgctggcgatttgagggcacgggttccggagtgatagttaccattacacctccggccaaggaggaatccaccccaccccc

SEQ ID NO: 177 - DNA, 203 bp, synthetic construct; identical to SEQ ID NO: 176
  misc_feature 1..203: note = Description of Artificial Sequence: Synthetic polynucleotide
  misc_feature 1..203: note = MG64-46 effector putative tracrRNA
  source 1..203: mol_type = other DNA

SEQ ID NO: 178 - DNA, 310 bp, synthetic construct
  misc_feature 1..310: note = Description of Artificial Sequence: Synthetic polynucleotide
  misc_feature 1..310: note = MG64-49 effector putative tracrRNA
  source 1..310: mol_type = other DNA
gattgcgcctcgatcgatgctctatgagccgctcgatcgtagaaaaatgggtgagtttgattatctacttcgttagataatgctgctttccgaccctggcattctgtccgcccttgaagctgcttctcatggactagcgtaagctcgttggtaagaaggaaaagtcataatttaaagtcacgtctttctagtatgacataggtgcgctcccacgcaatatagggttcagcttttattttataaaagtagagactttcctctagtgacagtgccgaaatgaccccgtgcgaggggtaactacctaagtttt

SEQ ID NO: 179 - DNA, 227 bp, synthetic construct
  misc_feature 1..227: note = Description of Artificial Sequence: Synthetic polynucleotide
  misc_feature 1..227: note = MG64-51 effector putative tracrRNA
  source 1..227: mol_type = other DNA
aacagcgccgcagttcatgtttgttataaacctctgtactgcgataaatgcgggttagtttgactgttgtgagacagtcttgctttctgaccctagtagctgcccaccttgatgctgctgttcccagtgaacaggaataaggtgcgcccccagtaatagaggtgcgggtttaccgcagtggtggctactgaatcacctccgactaaggaggaatccaccttaattat

SEQ ID NO: 180 - DNA, 380 bp, synthetic construct
  misc_feature 1..380: note = Description of Artificial Sequence: Synthetic polynucleotide
  misc_feature 1..380: note = MG64-52 effector putative tracrRNA
  source 1..380: mol_type = other DNA
accggggatcgtcctggtgaagggagagttgctttggatcggtcacaagcttttccttaactaattctcactgactgactaggatgatcgagggggttatgttttaccactgcaaggtggatactttcaaacccctgtggtagctgctcgctcctggtgaggtgccctgacacttcaccccactacagcaatgtgtgtggctgtctggttatgagagaagttagggcataggttcgttacctgcattcaatcataaattacgcgaccagataagtgagtatgatccgcaaggatctatatgtctttagcaaagaagtgcttctgctttgttactggcgtagggcatggttctctaaagtggctaccgaaccttcccaatt

SEQ ID NO: 181 - DNA, 257 bp, synthetic construct
  misc_feature 1..257: note = Description of Artificial Sequence: Synthetic polynucleotide
  misc_feature 1..257: note = MG64_56 effector putative tracrRNA
  source 1..257: mol_type = other DNA
aacagcgccgcagttcatgctcttctgagtctctgtactgtgataaatctgggttagtttaacggttgaaagaccgttttgctttctgaccctggtagctgctcgctcttgatgctgctgtcttttgacaggataggtgcgctcccagcaataaagagttaaagctgataaagcttgagccgttgtaaaacggtggggtttacctcagtggtggctactgaatcacccccttcgtcgggggaaccctcctaaatatt

SEQ ID NO: 182 - DNA, 246 bp, synthetic construct
  misc_feature 1..246: note = Description of Artificial Sequence: Synthetic polynucleotide
  misc_feature 1..246: note = MG108-1 effector putative tracrRNA
  source 1..246: mol_type = other DNA
tttagcgcacttgttcgagttcgtttgaactgaacaagggtaagtatgggccagtttaattgctttccgtcccaggatagctgccagcttctaccgtaggttcgtcctgcaagtgatgctaagtcgcgcctagcatcaaggagctatgtcttgattgtcttgggtgtccgccctggatgagttgaggtgtagatgcttctatcatggcagctactaaacgccccaagcaaggggaacccatcttta

SEQ ID NO: 183 - DNA, 223 bp, synthetic construct
  misc_feature 1..223: note = Description of Artificial Sequence: Synthetic polynucleotide
  misc_feature 1..223: note = MG108-2 effector putative tracrRNA
  source 1..223: mol_type = other DNA
aatagcgccgtagttcatgcttgctaaagcctctgaattgcgaaaagtccgggttagtgctgtcggcagacagcgttgctttctgaccctggtagctgcccaccccgatgctgctgtcccttgcagacaggaaccaggtgcgcccccagtaataagggtgtgggtttaccacagtggtggctactgaatcacctccgagcaaggaggaatccaccttaactat

SEQ ID NO: 184 - DNA, 41 bp, synthetic construct
  misc_feature 1..41: note = Description of Artificial Sequence: Synthetic oligonucleotide
  misc_feature 1..41: note = MG64-9 effector target CRISPR repeat
  source 1..41: mol_type = other DNA
gtctttcatcctatctcgcgccagatcgcttcctgcaaccc

SEQ ID NO: 185 - DNA, 37 bp, synthetic construct
  misc_feature 1..37: note = Description of Artificial Sequence: Synthetic oligonucleotide
  misc_feature 1..37: note = MG64-12 effector target CRISPR repeat
  source 1..37: mol_type = other DNA
gttgcaagcgcctccttggctgttggtgggtggaaag

SEQ ID NO: 186 - DNA, 37 bp, synthetic construct
  misc_feature 1..37: note = Description of Artificial Sequence: Synthetic oligonucleotide
  misc_feature 1..37: note = MG64-14 effector target CRISPR repeat
  source 1..37: mol_type = other DNA
gttgcaatcgccttcccagagatgggtgggctgaaag

SEQ ID NO: 187 - DNA, 38 bp, synthetic construct
  misc_feature 1..38: note = Description of Artificial Sequence: Synthetic oligonucleotide
  misc_feature 1..38: note = MG64-15 effector target CRISPR repeat
  source 1..38: mol_type = other DNA
gttacaattaccctcccagcgttgggtgggttgaaagg

SEQ ID NO: 188 - DNA, 39 bp, synthetic construct
  misc_feature 1..39: note = Description of Artificial Sequence: Synthetic oligonucleotide
  misc_feature 1..39: note = MG64-16 effector target CRISPR repeat
  source 1..39: mol_type = other DNA
aagttgcatccgctttccagcaaccagggcgggtgaaag

SEQ ID NO: 189 - DNA, 37 bp, synthetic construct
  misc_feature 1..37: note = Description of Artificial Sequence: Synthetic oligonucleotide
  misc_feature 1..37: note = MG64-17 effector target CRISPR repeat
  source 1..37: mol_type = other DNA
gttgcatccgctttccagcaaccagggcgggtgaaag

SEQ ID NO: 190 - DNA, 33 bp, synthetic construct
  misc_feature 1..33: note = Description of Artificial Sequence: Synthetic oligonucleotide
  misc_feature 1..33: note = MG64-19 effector target CRISPR repeat
  source 1..33: mol_type = other DNA
gtacccaaagccttttttccttaagcctatccg

SEQ ID NO: 191 - DNA, 27 bp, synthetic construct
  misc_feature 1..27: note = Description of Artificial Sequence: Synthetic oligonucleotide
  misc_feature 1..27: note = MG64-25 effector target CRISPR repeat
  source 1..27: mol_type = other DNA
gtttcaaccgccatcccagctaggggt

SEQ ID NO: 192 - DNA, 38 bp, synthetic construct
  misc_feature 1..38: note = Description of Artificial Sequence: Synthetic oligonucleotide
  misc_feature 1..38: note = MG64-27 effector target CRISPR repeat
  source 1..38: mol_type = other DNA
agttgcatccgctttccagcaaccagggcgggtgaaag

SEQ ID NO: 193 - DNA, 38 bp, synthetic construct
  misc_feature 1..38: note = Description of Artificial Sequence: Synthetic oligonucleotide
  misc_feature 1..38: note = MG64-28 effector target CRISPR repeat
  source 1..38: mol_type = other DNA
gttgcatctgcttttcagcaactagggcgggggaaagc

SEQ ID NO: 194 - DNA, 37 bp, synthetic construct
  misc_feature 1..37: note = Description of Artificial Sequence: Synthetic oligonucleotide
  misc_feature 1..37: note = MG64-29 effector target CRISPR repeat
  source 1..37: mol_type = other DNA
ggcgcgatcgcctttatgggtacgggcaagttgaaag

SEQ ID NO: 195 - DNA, 41 bp, synthetic construct
  misc_feature 1..41: note = Description of Artificial Sequence: Synthetic oligonucleotide
  misc_feature 1..41: note = MG64-32 effector target CRISPR repeat
  source 1..41: mol_type = other DNA
aagttgcatccgctttccagcaaccagggcgggtgaaagtt

SEQ ID NO: 196 - DNA, 37 bp, synthetic construct
  misc_feature 1..37: note = Description of Artificial Sequence: Synthetic oligonucleotide
  misc_feature 1..37: note = MG64-44 effector target CRISPR repeat
  source 1..37: mol_type = other DNA
gttgcctcccgcttcgaggcacgggaacgattgaaag

SEQ ID NO: 197 - DNA, 37 bp, synthetic construct; identical to SEQ ID NO: 196
  misc_feature 1..37: note = Description of Artificial Sequence: Synthetic oligonucleotide
  misc_feature 1..37: note = MG64-46 effector target CRISPR repeat
  source 1..37: mol_type = other DNA

SEQ ID NO: 198 - DNA, 43 bp, synthetic construct
  misc_feature 1..43: note = Description of Artificial Sequence: Synthetic oligonucleotide
  misc_feature 1..43: note = MG64-49 effector target CRISPR repeat
  source 1..43: mol_type = other DNA
gttgcaacactccctgactgcctgacacaaatgcctcgaaagc

SEQ ID NO: 199 - DNA, 34 bp, synthetic construct
  misc_feature 1..34: note = Description of Artificial Sequence: Synthetic oligonucleotide
  misc_feature 1..34: note = MG64-52 effector target CRISPR repeat
  source 1..34: mol_type = other DNA
gtcgcaatgactattttggcttggggcggaatga

SEQ ID NO: 200 - DNA, 37 bp, synthetic construct
  misc_feature 1..37: note = Description of Artificial Sequence: Synthetic oligonucleotide
  misc_feature 1..37: note = MG64-51 effector target CRISPR repeat
  source 1..37: mol_type = other DNA
gtttcaacacccctcccgaagtggggcgggttgaaag

SEQ ID NO: 201 - RNA, 261 nt, synthetic construct; identical to SEQ ID NO: 111
  misc_feature 1..261: note = Description of Artificial Sequence: Synthetic polynucleotide
  misc_feature 1..261: note = MG64-6 active effector sgRNA
  source 1..261: mol_type = other RNA
  misc_difference 239..261: note = any nucleotide

SEQ ID NO: 202 - RNA, 263 nt, synthetic construct; identical to SEQ ID NO: 112
  misc_feature 1..263: note = Description of Artificial Sequence: Synthetic polynucleotide
  misc_feature 1..263: note = MG64-2 effector sgRNA
  source 1..263: mol_type = other RNA
  misc_difference 241..263: note = any nucleotide

SEQ ID NO: 203 - RNA, 265 nt, synthetic construct; identical to SEQ ID NO: 113
  misc_feature 1..265: note = Description of Artificial Sequence: Synthetic polynucleotide
  misc_feature 1..265: note = MG64-4 effector sgRNA
  source 1..265: mol_type = other RNA
  misc_difference 243..265: note = any nucleotide

SEQ ID NO: 204 - RNA, 276 nt, synthetic construct; identical to SEQ ID NO: 114
  misc_feature 1..276: note = Description of Artificial Sequence: Synthetic polynucleotide
  misc_feature 1..276: note = MG64-7 effector sgRNA
  source 1..276: mol_type = other RNA
  misc_difference 254..276: note = any nucleotide

SEQ ID NO: 205 - RNA, 246 nt, synthetic construct; identical to SEQ ID NO: 115
  misc_feature 1..246: note = Description of Artificial Sequence: Synthetic polynucleotide
  misc_feature 1..246: note = MG108-1 effector sgRNA 1
  source 1..246: mol_type = other RNA
  misc_difference 224..246: note = any nucleotide

SEQ ID NO: 206 - RNA, 274 nt, synthetic construct; identical to SEQ ID NO: 116
  misc_feature 1..274: note = Description of Artificial Sequence: Synthetic polynucleotide
  misc_feature 1..274: note = MG108-1 effector sgRNA 2
  source 1..274: mol_type = other RNA
  misc_difference 252..274: note = any nucleotide

SEQ ID NO: 207 - RNA, 60 nt, synthetic construct; identical to SEQ ID NO: 121
  misc_feature 1..60: note = Description of Artificial Sequence: Synthetic oligonucleotide
  misc_feature 1..60: note = MG110-1-6 effector crRNA
  source 1..60: mol_type = other RNA
  misc_difference 9..40: note = any nucleotide

SEQ ID NO: 208 - RNA, 59 nt, synthetic construct; identical to SEQ ID NO: 122
  misc_feature 1..59: note = Description of Artificial Sequence: Synthetic oligonucleotide
  misc_feature 1..59: note = MG110-2-6 effector crRNA
  source 1..59: mol_type = other RNA
  misc_difference 7..38: note = any nucleotide

SEQ ID NO: 209 - RNA, 227 nt, synthetic construct
  misc_feature 1..227: note = Description of Artificial Sequence: Synthetic polynucleotide
  misc_feature 1..227: note = MG64-4 effector putative tracrRNA
  source 1..227: mol_type = other RNA
aatagcgccgcttgttcatgcgcgaaagcgtctctgaacagtgtaaatgtgggttagtttgactgtcgtgaagacggtcttgctttctgaccctggtagctgcccaccttgaagctgctgtctcttgtagacaggaatcaggtgcgcccccagtaatataggtgcgggtttaccgcagtggtggctacccaatcacctccgagcaaggaggaacccaccttaattat

SEQ ID NO: 210 - RNA, 228 nt, synthetic construct
  misc_feature 1..228: note = Description of Artificial Sequence: Synthetic polynucleotide
  misc_feature 1..228: note = MG64-7 effector putative tracrRNA
  source 1..228: mol_type = other RNA
aacagcgccgcagttcatgcgtcttatggcgcctctgtgctgtgcaaaatgtgggttagtttgactgttggaagacagtcttgctttctgaccctggtagctgcccaccttgaagctgctatcccttgtggataggaatcaggtgcgcccccagtaatagaggtgcgggtttaccgcagtggtggctaccgaatcacctccgagcaaggaggaacccaccttaattat

SEQ ID NO: 211 - RNA, 328 nt, synthetic construct
  misc_feature 1..328: note = Description of Artificial Sequence: Synthetic polynucleotide
  misc_feature 1..328: note = MG64-9 effector putative tracrRNA
  source 1..328: mol_type = other RNA
gctgaagttagtctctggatactgaagtcagtgtctagggactgaagtaaggggagctttacccgcaagggatactttcgaccccctgtagctgcccgctcctggtggggtgccctgacacccgcctcattacagcaatgtatgactgtctgggctaatgaaagaaggattagggacgcaggttcacgacctacttcaatcatagtctgtgcaacccagataagtgagtatgacccgcaagggtctaaacgcctttagtagaatggttcttccagtttgttaagggcagggcttggttctccaaggtggctacgaattcttctcgatt

SEQ ID NO: 212 - RNA, 187 nt, synthetic construct
  misc_feature 1..187: note = Description of Artificial Sequence: Synthetic polynucleotide
  misc_feature 1..187: note = MG64-10 effector putative tracrRNA
  source 1..187: mol_type = other RNA
ggtagcgctggagccacatagttcataagctcacgcttcttggacttcctgtgttctctaaaacgggttctgttttacccttaccaagggatactttcagatccgagtagctgcaagctcatggcggagtgtcccctgacgctttgccaccgtcatagcgatgtgatggccgtctggcgtatgaacg

SEQ ID NO: 213 - RNA, 326 nt, synthetic construct
  misc_feature 1..326: note = Description of Artificial Sequence: Synthetic polynucleotide
  misc_feature 1..326: note = MG64-12 effector putative tracrRNA
  source 1..326: mol_type = other RNA
aatagcgcccctaatcaaatcgaaggcgtttgaggacggggaaaatgggtaagtttccatcgaaaggtggttcttttcagccctatgtagtcccactcccttgtggagtgttagcatctgaggtgcctagcacagcaaagtctctcaggagacggagtcaaagctggggaacaagtattacgaacgaatctcgcgattctattagtatacgtaggtcgctcccatgcaacaagatgctcatctctcagcaatgagggtagggaacgtatcccaaacactacatgaatagctcgtcgggcttgcccggtgagtatccaccaagcctt

SEQ ID NO: 214 - RNA, 311 nt, synthetic construct
  misc_feature 1..311: note = Description of Artificial Sequence: Synthetic polynucleotide
  misc_feature 1..311: note = MG64-13 effector putative tracrRNA
  source 1..311: mol_type = other RNA
cgctaggtttgccttttcgcgcgctgctgaagcagtgaaaagttccgacagtttaaaagttctaacacttttactgctttccgtcgggagtagttgtccgcttctgcgttgttgagaccgaagtttgtccaggtgataggcagaacgttgatgcaacaggtagatagtcgcgctctatcaggaagctgttctagttattagtgttcgtgcactaataaagaggatacagggatacatgtggttgtgtccagcaatggcacaacagggccactactcgaagccccgagcaagggttgagcctacccaaattt

SEQ ID NO: 215 - RNA, 342 nt, synthetic construct
  misc_feature 1..342: note = Description of Artificial Sequence: Synthetic polynucleotide
  misc_feature 1..342: note = MG64-14 effector putative tracrRNA
  source 1..342: mol_type = other RNA
aacagcgccgcagtttaagctctatagccgctgaactgtgaaaaatgtgggtcagtttggtcgttgcaagacgatcgtgctttccgaccctagtaactgtccgctcactgactgccatcctggggcaaatcttcaaattttgtgtatttgtgtggggatggaaagctgcattagtcgattctcttcctccaatgtagcgtaggtgcgcacccagcagaagtgagttaagccttcacaatgtggaggtacagaagcatcatctctccattttttggtgtagatggtgtgactgaagtggtagttaccgaatcgcccctgatcaagggggagccctccataatt

SEQ ID NO: 216 - RNA, 297 nt, synthetic construct
  misc_feature 1..297: note = Description of Artificial Sequence: Synthetic polynucleotide
  misc_feature 1..297: note = MG64-15 effector putative tracrRNA
  source 1..297: mol_type = other RNA
aatgacgtttgtttctgtgcgcgctgctgaagcagtgaaaaatacggacagtctaatttgctttccgtccggagtagttgtccgcttctgcgttgttggaaccgaagtttgtccaggtgataggcggagtgtttcggcaacagatggagagtcgcacctctatcagggggcagttttagtggttagtgtttagcgcactatcactaggatacagggatacatgtggtagtgaccagagatggcactgcgggaccgctactcgaagccccgaacaagggttgagcttacccaaatctt

SEQ ID NO: 217 - RNA, 226 nt, synthetic construct
  misc_feature 1..226: note = Description of Artificial Sequence: Synthetic polynucleotide
  misc_feature 1..226: note = MG64-16 effector putative tracrRNA
  source 1..226: mol_type = other RNA
aatagcgccgcagtttaagctcagcaagcctctggactgcgaaaagtatggggtagtttgaccgtcggtaaacggttgtgctttctgcccctggcgactgcccaccccgatgctgtcgatttcttaactgggaatcgagatgaggtgcgcccccagcaaaagggaacgggtttactggagtggtggtcgccgaatcacccccgagcaagggggactcgtcctttgc

SEQ ID NO: 218 - RNA, 227 nt, synthetic construct
  misc_feature 1..227: note = Description of Artificial Sequence: Synthetic polynucleotide
  misc_feature 1..227: note = MG64-17 effector putative tracrRNA
  source 1..227: mol_type = other RNA
aatagcgccgcagtttaagctcaatgagcctcttgactgcgaacagtatggggtagtttgaccggcggtaaccggttgtgctttctgcccctggcgactgcccaccccgatgctgtcgatttctcaaccgggaatcgagaatcaggtgcgcccccagcaagagggaacgggtttactggagtggtggtcgccgaatcacccccgagcaagggggactcgtcctttgc

SEQ ID NO: 219 - RNA, 227 nt, synthetic construct
  misc_feature 1..227: note = Description of Artificial Sequence: Synthetic polynucleotide
  misc_feature 1..227: note = MG64-19 effector putative tracrRNA
  source 1..227: mol_type = other RNA
aatagcgccgcagtttaagctcagcaagcctctggactgcgaaaagtatggggtagtttgaccgtcggtaaacggttgtgctttctgcccctggcgactgcccaccccgatgctgtcgatttctcaaccgggaatcgagaatcaggtgcgcccccagcaagagggaacgggtttactggagtggtggtcgccgaatcacccccgagcaagggggactcgtcctttgc

SEQ ID NO: 220 - RNA, 211 nt, synthetic construct
  misc_feature 1..211: note = Description of Artificial Sequence: Synthetic polynucleotide
  misc_feature 1..211: note = MG64-20 effector putative tracrRNA
  source 1..211: mol_type = other RNA
aatagcgccgcagtttaagctcaatgagcctcttgactgcgaacagtatggggtagtttgaccggcggtaaccggttgtgctttctgcccctggcgactgcccaccccgatgctgtcgatttctcaaccgggaatcgagaatcaggtgcgcccccagcaagagggaacgggtttactggagtggtggtcgccgaatcacccccgagcaagg

SEQ ID NO: 221 - RNA, 222 nt, synthetic construct
  misc_feature 1..222: note = Description of Artificial Sequence: Synthetic polynucleotide
  misc_feature 1..222: note = MG64-21 effector putative tracrRNA
  source 1..222: mol_type = other RNA
aatagcgccgcagttcatgcttctttgaagcctctgtgctgtgcaaaatgtgggttagtttggctgttgaagaaacagccttgctttctgaccctggtagctgtccaccctgaagctgctatcccctgtggataggataggtgcgcccccagcaataggggagcgggtataccgcagtggtggctactgaatcacctccaagcaaggaggaatccactttat

SEQ ID NO: 222 - RNA, 222 nt, synthetic construct; identical to SEQ ID NO: 221
  misc_feature 1..222: note = Description of Artificial Sequence: Synthetic polynucleotide
  misc_feature 1..222: note = MG64-22 effector putative tracrRNA
  source 1..222: mol_type = other RNA

SEQ ID NO: 223 - RNA, 248 nt, synthetic construct
  misc_feature 1..248: note = Description of Artificial Sequence: Synthetic polynucleotide
  misc_feature 1..248: note = MG64-24 effector putative tracrRNA
  source 1..248: mol_type = other RNA
aaaagcgccgtagaacatgctcacgcctctgttctgcgaaaaattagggtttgtttggctgtctgacagcagtcttactttctgtccctagaatctgaccactccgatgctgctgttgtaagtgaacttgattgagctcggacaccatgcaacaggataaggggcgcacccagcaagagaggacggacttaccgtagtgttggcttctgaagcaactccgaccaaggagtagtccatgcattcatcat

SEQ ID NO: 224 - RNA, 277 nt, synthetic construct
  misc_feature 1..277: note = Description of Artificial Sequence: Synthetic polynucleotide
  misc_feature 1..277: note = MG64-25 effector putative tracrRNA
  source 1..277: mol_type = other RNA
aatcgcgccgcacattcatgttccttgagaacctctgaattgcgaaagtgtgggctagtttgttcgcttgatgcgaatgtgctttctggccctggtagctgtccgccctgatgctgatttctacgggtaactgtaggaatgattaactcgttctatagacaggtttcgtgctttctattgtaacggggtcggtgcgctcccagcaataggggtgtgggtctactacagtgatggctactgaatcacctccgagcaaggaggaatccaccttaacttt

SEQ ID NO: 225 - RNA, 226 nt, synthetic construct
  misc_feature 1..226: note = Description of Artificial Sequence: Synthetic polynucleotide
  misc_feature 1..226: note = MG64-27 effector putative tracrRNA
  source 1..226: mol_type = other RNA
aatagcgccgcagtttaagctcagcaagcctctggactgcgaaaagtatggggtagtttgaccgtcggtaaacggttgtgctttctgcccctggcgactgcccaccccgatgctgtcgatttcttaactgggaatcgagatgaggtgcgcccccagcaagagggaacgggtttactggagtggtggtcgccgaatcacccccgagcaagggggactcgtcctttgc

SEQ ID NO: 226 - RNA, 226 nt, synthetic construct; identical to SEQ ID NO: 225
  misc_feature 1..226: note = Description of Artificial Sequence: Synthetic polynucleotide
  misc_feature 1..226: note = MG64-28 effector putative tracrRNA
  source 1..226: mol_type = other RNA

SEQ ID NO: 227 - RNA, 255 nt, synthetic construct
  misc_feature 1..255: note = Description of Artificial Sequence: Synthetic polynucleotide
  misc_feature 1..255: note = MG64-29 effector putative tracrRNA
  source 1..255: mol_type = other RNA
tagcgcacgaacctgactagcgtcattacgatgcgagttctggaaatgggacagtttcattgctttccgtccctggcactgcctgcttactcacgaccacaaggagacaattcagcgctgtgagttcgcgattctctactgtttatgacaaactaacgtttgtctggcagtacagcaagattgtgtatcagaccatgtttggcgaagatacatggcaggccgaatcgccaatgaaaccagcggacttcccctaat

SEQ ID NO: 228 - RNA, 226 nt, synthetic construct
  misc_feature 1..226: note = Description of Artificial Sequence: Synthetic polynucleotide
  misc_feature 1..226: note = MG64-32 effector putative tracrRNA
  source 1..226: mol_type = other RNA
aatagcgccgcagtttaagctcaatgagcctcttgactgcgaacagtatggggtagtttgaccggcggtaaccggttgtgctttctgcccctggcgactgcccaccccgatgctgtcgatttcttaactgggaatcgagatgaggtgcgcccccagcaagagggaacgggtttactggagtggtggtcgccgaatcacccccgagcaagggggactcgtcctttgc

SEQ ID NO: 229 - RNA, 203 nt, synthetic construct
  misc_feature 1..203: note = Description of Artificial Sequence: Synthetic polynucleotide
  misc_feature 1..203: note = MG64-44 effector putative tracrRNA
  source 1..203: mol_type = other RNA
tctagcgccgcagctcatgtcagcaatggccaatgtgttgtgctaaatgcgagctagtttgactgcctgctaagcagtcttgctttctggctcaggtgactatccacccaaaggtcgttggtgcgctggcgatttgagggcacgggttccggagtgatagttaccattacacctccggccaaggaggaatccaccccaccccc

SEQ ID NO: 230 - RNA, 203 nt, synthetic construct; identical to SEQ ID NO: 229
  misc_feature 1..203: note = Description of Artificial Sequence: Synthetic polynucleotide
  misc_feature 1..203: note = MG64-46 effector putative tracrRNA
  source 1..203: mol_type = other RNA

SEQ ID NO: 231 - RNA, 310 nt, synthetic construct
  misc_feature 1..310: note = Description of Artificial Sequence: Synthetic polynucleotide
  misc_feature 1..310: note = MG64-49 effector putative tracrRNA
  source 1..310: mol_type = other RNA
gattgcgcctcgatcgatgctctatgagccgctcgatcgtagaaaaatgggtgagtttgattatctacttcgttagataatgctgctttccgaccctggcattctgtccgcccttgaagctgcttctcatggactagcgtaagctcgttggtaagaaggaaaagtcataatttaaagtcacgtctttctagtatgacataggtgcgctcccacgcaatatagggttcagcttttattttataaaagtagagactttcctctagtgacagtgccgaaatgaccccgtgcgaggggtaactacctaagtttt

SEQ ID NO: 232 - RNA, 227 nt, synthetic construct
  misc_feature 1..227: note = Description of Artificial Sequence: Synthetic polynucleotide
  misc_feature 1..227: note = MG64-51 effector putative tracrRNA
  source 1..227: mol_type = other RNA
aacagcgccgcagttcatgtttgttataaacctctgtactgcgataaatgcgggttagtttgactgttgtgagacagtcttgctttctgaccctagtagctgcccaccttgatgctgctgttcccagtgaacaggaataaggtgcgcccccagtaatagaggtgcgggtttaccgcagtggtggctactgaatcacctccgactaaggaggaatccaccttaattat

SEQ ID NO: 233 - RNA, 380 nt, synthetic construct
  misc_feature 1..380: note = Description of Artificial Sequence: Synthetic polynucleotide
  misc_feature 1..380: note = MG64-52 effector putative tracrRNA
  source 1..380: mol_type = other RNA
accggggatcgtcctggtgaagggagagttgctttggatcggtcacaagcttttccttaactaattctcactgactgactaggatgatcgagggggttatgttttaccactgcaaggtggatactttcaaacccctgtggtagctgctcgctcctggtgaggtgccctgacacttcaccccactacagcaatgtgtgtggctgtctggttatgagagaagttagggcataggttcgttacctgcattcaatcataaattacgcgaccagataagtgagtatgatccgcaaggatctatatgtctttagcaaagaagtgcttctgctttgttactggcgtagggcatggttctctaaagtggctaccgaaccttcccaatt

SEQ ID NO: 234 - RNA, 257 nt, synthetic construct
  misc_feature 1..257: note = Description of Artificial Sequence: Synthetic polynucleotide
  misc_feature 1..257: note = MG64-56 effector putative tracrRNA
  source 1..257: mol_type = other RNA
aacagcgccgcagttcatgctcttctgagtctctgtactgtgataaatctgggttagtttaacggttgaaagaccgttttgctttctgaccctggtagctgctcgctcttgatgctgctgtcttttgacaggataggtgcgctcccagcaataaagagttaaagctgataaagcttgagccgttgtaaaacggtggggtttacctcagtggtggctactgaatcacccccttcgtcgggggaaccctcctaaatatt

SEQ ID NO: 235 - RNA, 246 nt, synthetic construct
  misc_feature 1..246: note = Description of Artificial Sequence: Synthetic polynucleotide
  misc_feature 1..246: note = MG108-1 effector putative tracrRNA
  source 1..246: mol_type = other RNA
tttagcgcacttgttcgagttcgtttgaactgaacaagggtaagtatgggccagtttaattgctttccgtcccaggatagctgccagcttctaccgtaggttcgtcctgcaagtgatgctaagtcgcgcctagcatcaaggagctatgtcttgattgtcttgggtgtccgccctggatgagttgaggtgtagatgcttctatcatggcagctactaaacgccccaagcaaggggaacccatcttta

SEQ ID NO: 236 - RNA, 223 nt, synthetic construct
  misc_feature 1..223: note = Description of Artificial Sequence: Synthetic polynucleotide
  misc_feature 1..223: note = MG108-2 effector putative tracrRNA
  source 1..223: mol_type = other RNA
aatagcgccgtagttcatgcttgctaaagcctctgaattgcgaaaagtccgggttagtgctgtcggcagacagcgttgctttctgaccctggtagctgcccaccccgatgctgctgtcccttgcagacaggaaccaggtgcgcccccagtaataagggtgtgggtttaccacagtggtggctactgaatcacctccgagcaaggaggaatccaccttaactat

SEQ ID NO: 237 - RNA, 41 nt, synthetic construct
  misc_feature 1..41: note = Description of Artificial Sequence: Synthetic oligonucleotide
  misc_feature 1..41: note = MG64-9 effector crRNA
  source 1..41: mol_type = other RNA
gtctttcatcctatctcgcgccagatcgcttcctgcaaccc

SEQ ID NO: 238 - RNA, 37 nt, synthetic construct
  misc_feature 1..37: note = Description of Artificial Sequence: Synthetic oligonucleotide
  misc_feature 1..37: note = MG64-12 effector crRNA
  source 1..37: mol_type = other RNA
gttgcaagcgcctccttggctgttggtgggtggaaag

SEQ ID NO: 239 - RNA, 37 nt, synthetic construct
  misc_feature 1..37: note = Description of Artificial Sequence: Synthetic oligonucleotide
  misc_feature 1..37: note = MG64-14 effector crRNA
  source 1..37: mol_type = other RNA
gttgcaatcgccttcccagagatgggtgggctgaaag

SEQ ID NO: 240 - RNA, 38 nt, synthetic construct
  misc_feature 1..38: note = Description of Artificial Sequence: Synthetic oligonucleotide
  misc_feature 1..38: note = MG64-15 effector crRNA
  source 1..38: mol_type = other RNA
gttacaattaccctcccagcgttgggtgggttgaaagg

SEQ ID NO: 241 - RNA, 39 nt, synthetic construct
  misc_feature 1..39: note = Description of Artificial Sequence: Synthetic oligonucleotide
  misc_feature 1..39: note = MG64-16 effector crRNA
  source 1..39: mol_type = other RNA
aagttgcatccgctttccagcaaccagggcgggtgaaag

SEQ ID NO: 242 - RNA, 37 nt, synthetic construct
  misc_feature 1..37: note = Description of Artificial Sequence: Synthetic oligonucleotide
  misc_feature 1..37: note = MG64-17 effector crRNA
  source 1..37: mol_type = other RNA
gttgcatccgctttccagcaaccagggcgggtgaaag

SEQ ID NO: 243 - RNA, 33 nt, synthetic construct
  misc_feature 1..33: note = Description of Artificial Sequence: Synthetic oligonucleotide
  misc_feature 1..33: note = MG64-19 effector crRNA
  source 1..33: mol_type = other RNA
gtacccaaagccttttttccttaagcctatccg

SEQ ID NO: 244 - RNA, 27 nt, synthetic construct
  misc_feature 1..27: note = Description of Artificial Sequence: Synthetic oligonucleotide
  misc_feature 1..27: note = MG64-25 effector crRNA
  source 1..27: mol_type = other RNA
gtttcaaccgccatcccagctaggggt

SEQ ID NO: 245 - RNA, 38 nt, synthetic construct
  misc_feature 1..38: note = Description of Artificial Sequence: Synthetic oligonucleotide
  misc_feature 1..38: note = MG64-27 effector crRNA
  source 1..38: mol_type = other RNA
agttgcatccgctttccagcaaccagggcgggtgaaag

SEQ ID NO: 246 - RNA, 38 nt, synthetic construct
  misc_feature 1..38: note = Description of Artificial Sequence: Synthetic oligonucleotide
  misc_feature 1..38: note = MG64-28 effector crRNA
  source 1..38: mol_type = other RNA
gttgcatctgcttttcagcaactagggcgggggaaagc

SEQ ID NO: 247 - RNA, 37 nt, synthetic construct
  misc_feature 1..37: note = Description of Artificial Sequence: Synthetic oligonucleotide
  misc_feature 1..37: note = MG64-29 effector crRNA
  source 1..37: mol_type = other RNA
ggcgcgatcgcctttatgggtacgggcaagttgaaag

SEQ ID NO: 248 - RNA, 41 nt, synthetic construct
  misc_feature 1..41: note = Description of Artificial Sequence: Synthetic oligonucleotide
  misc_feature 1..41: note = MG64-32 effector crRNA
  source 1..41: mol_type = other RNA
aagttgcatccgctttccagcaaccagggcgggtgaaagtt

SEQ ID NO: 249 - RNA, 37 nt, synthetic construct
  misc_feature 1..37: note = Description of Artificial Sequence: Synthetic oligonucleotide
  misc_feature 1..37: note = MG64-44 effector crRNA
  source 1..37: mol_type = other RNA
gttgcctcccgcttcgaggcacgggaacgattgaaag

SEQ ID NO: 250 - RNA, 37 nt, synthetic construct; identical to SEQ ID NO: 249
  misc_feature 1..37: note = Description of Artificial Sequence: Synthetic oligonucleotide
  misc_feature 1..37: note = MG64-46 effector crRNA
  source 1..37: mol_type = other RNA

SEQ ID NO: 251 - RNA, 43 nt, synthetic construct
  misc_feature 1..43: note = Description of Artificial Sequence: Synthetic oligonucleotide
  misc_feature 1..43: note = MG64-49 effector crRNA
  source 1..43: mol_type = other RNA
gttgcaacactccctgactgcctgacacaaatgcctcgaaagc

SEQ ID NO: 252 - RNA, 34 nt, synthetic construct
  misc_feature 1..34: note = Description of Artificial Sequence: Synthetic oligonucleotide
  misc_feature 1..34: note = MG64-52 effector crRNA
  source 1..34: mol_type = other RNA
gtcgcaatgactattttggcttggggcggaatga

SEQ ID NO: 253 - RNA, 37 nt, synthetic construct
  misc_feature 1..37: note = Description of Artificial Sequence: Synthetic oligonucleotide
  misc_feature 1..37: note = MG64-51 effector crRNA
  source 1..37: mol_type = other RNA
gtttcaacacccctcccgaagtggggcgggttgaaag

SEQ ID NO: 254 - RNA, 37 nt, synthetic construct
  misc_feature 1..37: note = Description of Artificial Sequence: Synthetic oligonucleotide
  misc_feature 1..37: note = MG64-56 effector crRNA
  source 1..37: mol_type = other RNA
gtttcaacaaccatcccagctaggggtgggttgaaag

SEQ ID NO: 255 - RNA, 37 nt, synthetic construct
  misc_feature 1..37: note = Description of Artificial Sequence: Synthetic oligonucleotide
  misc_feature 1..37: note = MG64-2 effector crRNA
  source 1..37: mol_type = other RNA
gtttcaacgaccatcccaactaggggtgggttgaaag

SEQ ID NO: 256 - RNA, 37 nt, synthetic construct
  misc_feature 1..37: note = Description of Artificial Sequence: Synthetic oligonucleotide
  misc_feature 1..37: note = MG64-4 effector crRNA
  source 1..37: mol_type = other RNA
gtttcaactttccttccagctagaggcgggttgaaag

SEQ ID NO: 257 - RNA, 37 nt, synthetic construct
  misc_feature 1..37: note = Description of Artificial Sequence: Synthetic oligonucleotide
  misc_feature 1..37: note = MG64-6 effector crRNA
  source 1..37: mol_type = other RNA
gtttcaaccaccatctcaactagggatgggttgaaag

SEQ ID NO: 258 - RNA, 38 nt, synthetic construct
  misc_feature 1..38: note = Description of Artificial Sequence: Synthetic oligonucleotide
  misc_feature 1..38: note = MG64-7 effector crRNA
  source 1..38: mol_type = other RNA
gtttcaacgccccttcaagctttgggcgggttgaaagc

SEQ ID NO: 259 - RNA, 36 nt, synthetic construct
  misc_feature 1..36: note = Description of Artificial Sequence: Synthetic oligonucleotide
  misc_feature 1..36: note = MG64-13 effector crRNA
  source 1..36: mol_type = other RNA
tggcaattgcccttccagtgttgggtgggttgaaag

SEQ ID NO: 260 - RNA, 37 nt, synthetic construct
  misc_feature 1..37: note = Description of Artificial Sequence: Synthetic oligonucleotide
  misc_feature 1..37: note = MG108-2 effector crRNA
  source 1..37: mol_type = other RNA
gtttcaacgaccatcccgacaaggggtgggttgaaag

SEQ ID NO: 261 - RNA, 37 nt, synthetic construct
  misc_feature 1..37: note = Description of Artificial Sequence: Synthetic oligonucleotide
  misc_feature 1..37: note = MG108-1 effector crRNA
  source 1..37: mol_type = other RNA
gttgcgatcgccgctccggtggcgatggggttgaaag